(19) United States
(12) Patent Application Publication
(10) Pub. No.: US 2026/0171192 A1
(43) Pub. Date: Jun. 18, 2026

(54) TECHNIQUES FOR DETECTING AMINO ACID VARIANTS USING NEXT-GENERATION PROTEIN SEQUENCING

(71) Applicant: Quantum-Si Incorporated, Branford, CT (US)

(72) Inventor: Douglas Pike, Guilford, CT (US)

(73) Assignee: Quantum-Si Incorporated, Branford, CT (US)

(21) Appl. No.: 19/423,279

(22) Filed: Dec. 17, 2025

Related U.S. Application Data
(60) Provisional application No. 63/735,163, filed on Dec. 17, 2024.

(51) Int. Cl. G16B 30/10; G01N 21/64; G16B 40/20

(52) U.S. Cl. CPC G16B 30/10; G01N 21/6428; G16B 40/20; G01N 2021/6439

(57) ABSTRACT

Described herein are techniques for detecting amino acid variants in peptides using data from a sequencing device. Sequencing data is generated from traces of light pulses output by the sequencing device from detecting light emissions by fluorescently tagged N-terminal amino acid (NAA) recognizers during sequencing of at least one peptide. The sequencing data comprising light pulse durations and inter-pulse durations between successive light pulses. The techniques generate read(s) each comprising a sequence of recognition segments indicating time periods in which fluorescently tagged NAA recognizers were binding to particular NAAs of the peptide, assign fluorescently tagged NAA recognizers to recognition segments, align read(s) to reference peptide sequences to obtain peptide alignments, and detect amino acid variants using the peptide alignments.

### RELATED APPLICATIONS

This application claims the benefit under 35 U.S.C. § 119(e) of U.S. Provisional Application No. 63/735,163 filed on Dec. 17, 2024, and is incorporated by reference herein.

### FIELD OF THE INVENTION

Techniques described herein relate to protein sequencing and proteomics analysis, and more particularly to techniques for detecting (e.g., quantifying) amino acid (AA) variants (e.g., single amino acid variants (SAAV)) in peptides using next-generation protein sequencing (NGSP) technology.

### BACKGROUND

Proteins are composed of amino acid residues arranged in specific sequences that determine protein structure and function. Variations in amino acid sequences, including single amino acid variants (SAAVs), can arise from genomic mutations, alternative splicing, post-translational modifications, and other biological processes. These protein variants, sometimes referred to as proteoforms, may have functional implications in biological systems and disease mechanisms. Understanding the diversity of protein variants present in biological samples has become an area of interest in proteomics research. Mass spectrometry has been a conventional approach for protein analysis and identification. Mass spectrometry techniques measure mass-to-charge ratios of ionized molecules to identify and characterize proteins and peptides.

Single-molecule protein sequencing technologies have emerged as approaches for analyzing proteins at the individual molecule level. These technologies can provide information about protein sequences by detecting interactions between individual peptide molecules and recognition agents. Some single-molecule approaches utilize fluorescently labeled recognition agents that bind to specific amino acid residues, generating optical signals that can be detected and analyzed. The binding and dissociation events between recognition agents and peptide molecules produce characteristic signal patterns that encode information about the amino acid composition of the peptides being analyzed.

### SUMMARY

Described herein are techniques for identifying amino acid variants in peptides using data obtained by a sequencing device. The techniques use sequencing data generated from traces of light pulses output by the sequencing device from detecting light emissions by fluorescently tagged N-terminal amino acid (NAA) recognizers in response to illumination during sequencing of peptide(s). The techniques use the sequencing data to generate reads that each include a sequence of recognition segments that each indicate a particular time period in which one or more of the fluorescently tagged NAA recognizers were binding to a particular NAA of the peptide(s). The techniques align the reads to reference peptide sequence(s) of peptide variant(s). The techniques use alignment(s) to perform detection of amino acid variant(s) (e.g., by identifying positions of variation, AA variant(s) at position(s), and/or quantifying AA variant(s) at position(s)).

In some embodiments, the techniques described herein relate to a method for detecting amino acid variants in peptides using data obtained by a sequencing device, the method including: using at least one computer hardware processor to perform: obtaining sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assigning, to recognition segments in the plurality of reads, fluorescently tagged NAA recognizers determined to be binding in the recognition segments at least in part by using: (1) the light pulse durations, and (3) the inter-pulse durations; and detecting one or more amino acid variants in the at least one peptide using the plurality of reads and an assignment of the fluorescently tagged NAA recognizers to the recognition segments of the plurality of reads.

In some embodiments, the techniques described herein relate to a system for identifying amino acid variants in peptides using data obtained by a sequencing device, the system including: the sequencing device, the sequencing device configured to obtain sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to: generate, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assign, to recognition segments in the plurality of reads, fluorescently tagged NAA recognizers determined to be binding in the recognition segments at least in part by using: (1) the light pulse durations, and (3) the inter-pulse durations; and detect one or more amino acid variants in the at least one peptide using the plurality of reads and an assignment of the fluorescently tagged NAA recognizers to the recognition segments of the plurality of reads.

In some embodiments, the techniques described herein relate to a non-transitory computer-readable storage medium storing instructions that, when executed by at least one computer hardware processor, cause the at least one computer hardware processor to perform a method for identifying amino acid variants in peptides using data obtained by a sequencing device, the method including: obtaining sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assigning, to recognition segments in the plurality of reads, fluorescently tagged NAA recognizers determined to be binding in the recognition segments at least in part by using: (1) the light pulse durations, and (3) the inter-pulse durations; and detecting one or more amino acid variants in the at least one peptide using the plurality of reads and an assignment of the fluorescently tagged NAA recognizers to the recognition segments of the plurality of reads.

In some embodiments, the techniques described herein relate to a method for generating reads using data obtained by a sequencing device from detecting light emissions by fluorescently tagged N-terminal amino acid (NAA) recognizers during sequencing of at least one peptide, the method including: using at least one computer hardware processor to perform: obtaining sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.

In some embodiments, the techniques described herein relate to a system for generating reads using data obtained by a sequencing device from detecting light emissions by fluorescently tagged N-terminal amino acid (NAA) recognizers during sequencing of at least one peptide, the method including: a sequencing device, the sequencing device configured to obtain sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to perform: generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.

In some embodiments, the techniques described herein relate to a non-transitory computer-readable storage medium storing instructions that, when executed by at least one computer hardware processor, cause the at least one computer hardware processor to perform a method for generating reads using data obtained by a sequencing device from detecting light emissions by fluorescently tagged N-terminal amino acid (NAA) recognizers during sequencing of at least one peptide, the method including: obtaining sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.

In some embodiments, the techniques described herein relate to a method for identifying amino acid residues in peptides using data obtained by a sequencing device from detecting light emissions by fluorescently tagged N-terminal amino acid (NAA) recognizers during sequencing of the peptides, the method including: using at least one computer hardware processor to perform: obtaining sequencing data generated from traces of light pulses output by the sequencing device from detecting light emissions by fluorescently tagged NAA recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assigning, to recognition segments in the plurality of reads, fluorescently tagged NAA recognizers determined to be binding in the recognition segments.

In some embodiments, the techniques described herein relate to a system for identifying amino acid residues in peptides using data obtained by a sequencing device from detecting light emissions by fluorescently tagged N-terminal amino acid (NAA) recognizers during sequencing of the peptides, the method including: a sequencing device, the sequencing device configured to obtain sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to: generate, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assign, to recognition segments in the plurality of reads, fluorescently tagged NAA recognizers determined to be binding in the recognition segments.

In some embodiments, the techniques described herein relate to a non-transitory computer-readable storage medium storing instructions that, when executed by at least one computer hardware processor, cause the at least one computer hardware processor to perform a method for identifying amino acid residues in peptides using data obtained by a sequencing device from detecting light emissions by fluorescently tagged N-terminal amino acid (NAA) recognizers during sequencing of the peptides, the method including: obtaining sequencing data generated from traces of light pulses output by the sequencing device from detecting light emissions by fluorescently tagged NAA recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assigning, to recognition segments in the plurality of reads, fluorescently tagged NAA recognizers determined to be binding in the recognition segments.

### BRIEF DESCRIPTION OF THE FIGURES

FIG. 1A illustrates a block diagram of a sequencing data processing system for processing sequencing data generated by a peptide sequencing device from sequencing a peptide sample, according to some embodiments of the technology described herein.

FIG. 1B illustrates interaction between modules of the sequencing data processing system of FIG. 1A in processing sequencing data generated by the peptide sequencing device, according to some embodiments of the technology described herein.

FIG. 1C illustrates generation of reference alignment data by reference alignment data generation module of the sequencing data processing system of FIG. 1A, according to some embodiments of the technology described herein.

FIG. 2A illustrates example generation of a read by segmentation of a light pulse trace into recognition segments that form the read, according to some embodiments of the technology described herein.

FIG. 2B illustrates example segmentation of the light pulse trace into proto-recognition segments, according to some embodiments of the technology described herein.

FIG. 2C illustrates example segmentation of proto-recognition segments into recognition segments, according to some embodiments of the technology described herein.

FIG. 3A illustrates example training of a recognizer classification model using fluorescence data obtained from reads, according to some embodiments of the technology described herein.

FIG. 3B illustrates example assignment of recognizers to recognition segments using the recognizer classification model of FIG. 3A, according to some embodiments of the technology described herein.

FIG. 4A illustrates example alignment of a read to reference peptide sequences, according to some embodiments of the technology described herein.

FIG. 4B illustrates example matching of recognizers assigned to recognition segments of a read to amino acid residues of a reference peptide sequence, according to some embodiments of the technology described herein.

FIG. 5 illustrates example training of a machine learning model for prediction of pulse durations for amino acid residue motifs, according to some embodiments of the technology described herein.

FIG. 6 illustrates example amino acid variant detection performed to quantify amino acid variants in a peptide sample, according to some embodiments of the technology described herein.

FIG. 7 illustrates a flowchart of an example process for detecting amino acid variants in a peptide sample, according to some embodiments of the technology described herein.

FIG. 8 illustrates a flowchart of an example process for generating reads using sequencing data, according to some embodiments of the technology described herein.

FIG. 9 illustrates a flowchart of an example process for assigning fluorescently tagged NAA recognizers to recognition segments in reads, according to some embodiments of the technology described herein.

FIG. 10 illustrates a schematic overview of an example next-generation protein sequencing (NGPS) system and workflow, according to some embodiments of the technology described herein.

FIG. 11 illustrates a schematic diagram of an example peptide sequence design with amino acid variants at a specific position in the peptide sequence, according to some embodiments of the technology described herein.

FIG. 12 illustrates a graph of intensity as a function of time for a light pulse trace, according to some embodiments of the technology described herein.

FIG. 13 illustrates a scatter plot of classes of a recognizer classification model, according to some embodiments of the technology described herein.

FIG. 14 illustrates example alignments between a read and a reference peptide sequence, according to some embodiments of the technology described herein.

FIG. 15 illustrates an example technique for training a neural network for pulse duration prediction for amino acid motifs, according to some embodiments of the technology described herein.

FIG. 16 illustrates a flowchart for a variant detection workflow using sequencing data, according to some embodiments of the technology described herein.

FIG. 17 illustrates graphs showing kinetic properties of a peptide across multiple amino acid positions, according to some embodiments of the technology described herein.

FIG. 18 illustrates scatter plots comparing predicted ratios against expected ratios for variant titration datasets, according to some embodiments of the technology described herein.

FIG. 19 illustrates a multi-panel visualization showing kinetic properties of peptide variants, according to some embodiments of the technology described herein.

FIG. 20 illustrates a graph of log-likelihood as a function of log intensity offset for determining an intensity offset parameter, according to some embodiments of the technology described herein.

FIG. 21 illustrates a histogram of pulse bin ratios versus log-intensities with an initial guess dye caller overlaid, according to some embodiments of the technology described herein.

FIGS. 22A-22C illustrate histograms comparing un-biased and biased selection of pulses for dye caller training, according to some embodiments of the technology described herein.

FIGS. 23A-23C illustrate scatter plots showing progression of dye caller optimization from initial guess through intermediate optimization to final fitted dye caller, according to some embodiments of the technology described herein.

FIG. 24 illustrates a graph of a truncated quadratic scoring function used for alignment scoring, according to some embodiments of the technology described herein.

FIG. 25 illustrates a block diagram of a computing device 2500 that can be specially configured to implement some embodiments of the technology described herein.

### DETAILED DESCRIPTION

Described herein are techniques for processing sequencing data produced from NGPS sequencing of a peptide sample. The techniques utilize kinetics of NAA recognition to detect amino acid variants in a peptide sample. The techniques described herein may be used for identifying which variants are present in a sample and/or quantifying amounts of variants in the sample.

Protein sequencing and proteomics analysis present numerous technical challenges, particularly in the detection and characterization of protein variants. Proteoforms, which are protein variants arising from genomic, transcriptomic, and post-translational variation including alternative splicing and post-translational modifications, play roles in biological and disease mechanisms. Proteoforms play crucial roles in biological and disease mechanisms. However, conventional proteomics techniques can struggle to capture the full diversity and complexity of proteoforms.

Mass spectrometry (MS) is a conventional approach for protein analysis, but MS faces limitations in detecting certain types of protein variants. For example, single amino acid variants (SAAVs), which involve substitutions of individual amino acid residues within a protein sequence, may not be easily discerned by MS. As another example, isobaric amino acids, which have identical or nearly identical masses, present particular challenges for MS-based detection because MS relies on mass-to-charge ratios to distinguish between different molecular species. Similarly, highly similar proteoforms that differ by only subtle modifications or substitutions may be difficult to resolve using ensemble protein analysis methods that measure average properties across populations of molecules rather than individual molecular characteristics.

Next-generation protein sequencing (NGPS) technology addresses these technical problems by enabling real-time, single-molecule measurements of individual peptides. Unlike ensemble protein analysis methods, NGPS directly analyzes individual protein molecules, providing detailed information on modifications and variation at the single-molecule level. NGPS technology uses N-terminal amino acid (NAA) recognizers to detect individual amino acids in a peptide. Each NAA recognizer may be conjugated with a distinct fluorescent dye that has a characteristic intensity and fluorescence decay lifetime. The binding and dissociation of fluorescently tagged NAA recognizers to immobilized peptides may be monitored in real time by a sequencing device as individual on-off events, generating light pulse traces that encode information about the amino acid sequence of the peptide being analyzed.

A sequencing device performing NGPS generates sequencing data from light pulse traces obtained from detection of light emissions by fluorescently tagged NAA recognizers in response to illumination during sequencing. The light pulse traces may be further processed (e.g., by the sequencing device and/or another computing device) to generate sequencing data comprising properties of the light pulses (e.g., light pulse durations and inter-pulse durations). The light pulse properties may be governed by the kinetic properties of amino acids in the peptides. For example, light pulse durations may be governed by dissociation kinetics of the recognizer-peptide complex, while inter-pulse durations may be governed by the association kinetics of the recognizer-peptide complex. The inventors have recognized that these kinetic properties provide information that can be used to identify which amino acid is involved in a binding interaction at different points in the sequencing.

The inventors have developed techniques for processing NGSP data to generate reads that each comprise of a sequence of recognition segments. Each recognition segment indicates a particular time period in which one or more fluorescently tagged NAA recognizers (also referred to herein as a “recognizer(s)”) were binding to a particular NAA of a peptide being sequenced. The techniques use recognition segments to reconstruct amino acid sequence information from kinetic properties indicated by light pulses in the recognition segments. In particular, a system may assign fluorescently tagged NAA recognizers to recognition segments based on fluorescence data (e.g., fluorescence intensity and fluorescence decay) of light pulses in the recognition segments. The system uses the fluorescence data to classify recognition segments as corresponding to fluorescently tagged NAA recognizers that were binding during the recognition segments. The system can then use the assigned recognizers to identify amino acids corresponding to recognition segments (e.g., based on which amino acid(s) are associated with recognizers assigned to the recognition segments).

Some embodiments align reads to reference peptide sequences to obtain peptide alignments. The alignment process utilizes the fluorescently tagged NAA recognizers assigned to recognition segments, light pulse durations in the recognition segments, and inter-pulse durations to score candidate alignments and select alignments that best match the observed sequencing data to reference sequences. The system uses expected light emission properties (e.g., light pulse durations) for amino acid residues in reference peptide sequences to quantify a degree to which reads align to reference peptide sequences. The expected light emission properties are determined using a machine learning model trained, using empirical data, to predict light emission properties (e.g., light pulse duration). The trained machine learning model allows populating a reference dataset of expected light emission properties that can be used in alignment.

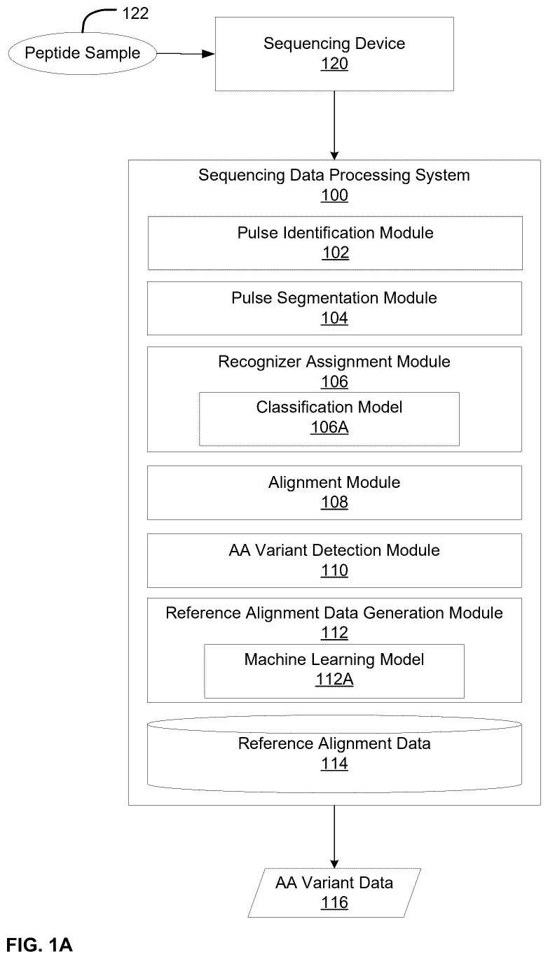

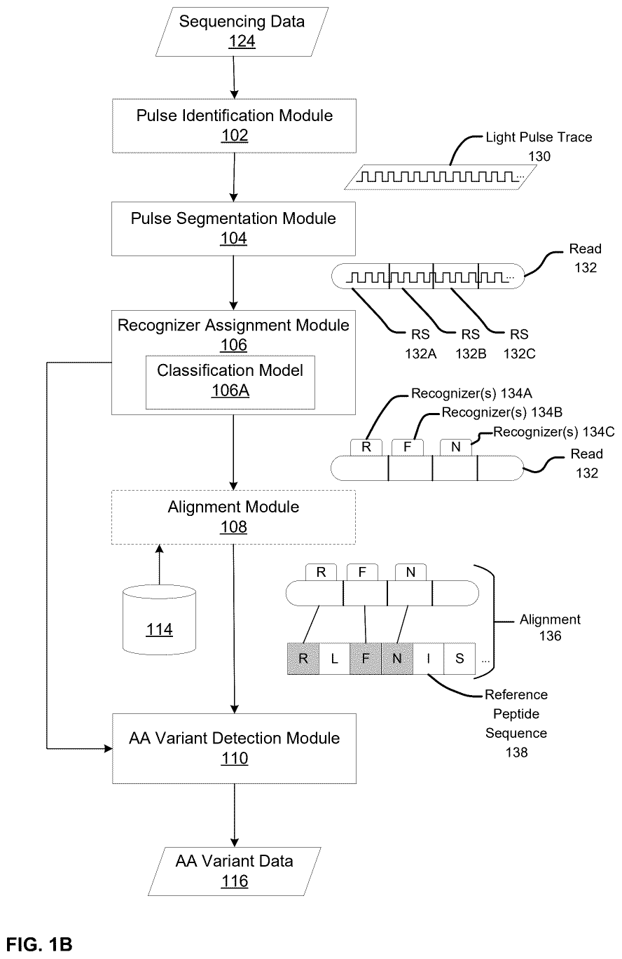

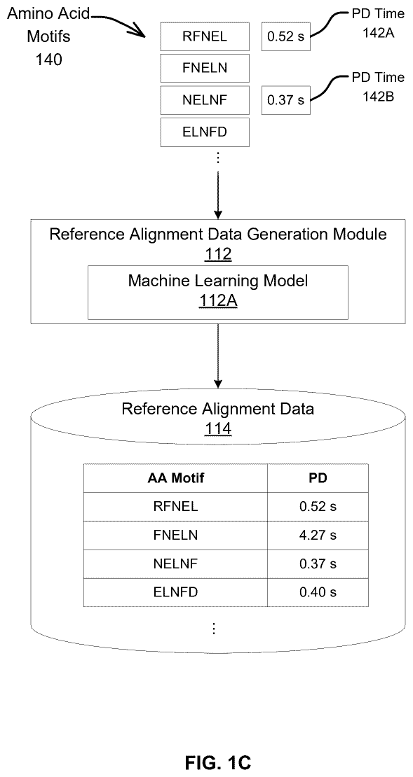

Some embodiments use the alignment of reads to reference peptide sequences to perform detection of amino acid variants in the sequenced peptides. By comparing observed kinetic properties associated with recognizer assignments to expected properties for different variant peptide sequences, the system distinguishes between peptide variants more effectively than conventional techniques. This is especially the case for variants that differ by single amino acid substitutions. For example, techniques described herein accurately detect SAAV mixture ratios in binary mixtures of synthetic peptides within a factor of ten of an expected value of the ratios. The techniques also recover expected variant ratios across diverse amino acid substitution types including residues that lack direct recognizers. Accordingly, embodiments described herein effectively used measured kinetic features to detect amino acid variants in peptide samples. FIG. 1A illustrates a sequencing data processing system 100 for processing sequencing data obtained from NGPS, according to some embodiments of the technology described herein. The sequencing data processing system 100 may receive input from a sequencing device 120, which sequences a peptide sample 122. The peptide sample 122 may have a collection of peptide molecules prepared for analysis. For example, the peptide sample 122 may be loaded at approximately 200 μM concentration for sequencing by the sequencing device 120. In some embodiments, the sequencing device 120 may be a NGPS instrument configured to perform single-molecule sequencing of peptides. The sequencing device 120 may include a semiconductor chip on which peptides from the peptide sample 122 are immobilized for sequencing. The sequencing device 120 may utilize fluorescently tagged NAA recognizers that reversibly bind to target amino acids at the N-terminus of immobilized peptides. The sequencing device 120 may include an illumination source (e.g., a laser or other illumination source) configured to excite the fluorescent dyes conjugated to the recognizers, and may include one or more detectors configured to detect light emissions from the fluorescent dyes in response to illumination.

In some embodiments, the sequencing device 120 may be configured to measure fluorescence decay. For example, the sequencing device 120 may measure fluorescence decay lifetime by sampling in multiple time periods following illumination (e.g., sample in two successive time periods following illumination by a laser). In some embodiments, the sequencing device 120 may utilize aminopeptidases that sequentially cleave N-terminal amino acids from the immobilized peptides, exposing successive amino acids for recognition by the recognizers. The binding and dissociation of the recognizers to the immobilized peptides may be monitored in real time by the sequencing device 120 as individual on-off events, generating light pulse traces that encode information about the amino acid sequence of the peptides being analyzed. In some embodiments, the sequencing device 120 may operate with a particular run time. For example, the sequencing device 120 may operate with a run time of approximately 10 hours for peptide sequencing and may use a particular frame rate (e.g., approximately 60 ms) for sampling signal data. Examples of sequencing devices that may be used as the sequencing device 120 may include the Quantum-Si Platinum instrument or another NGPS instrument capable of performing single-molecule peptide sequencing using fluorescently tagged NAA recognizers.

In some embodiments, the sequencing device 120 may include a photodetector configured to detect light emitted by fluorescently tagged NAA recognizers. An example of such a photodetector is described in U.S. Pat. No. 9,759,658, entitled “INTEGRATED DEVICE FOR TEMPORAL BINNING OF RECEIVED PHOTONS” and granted on Sep. 12, 2017.

As described therein, the photodetector may be configured to detect the arrival times of photons, which can allow for determining temporal characteristics of the light emitted by the recognizers. Detecting temporal characteristics of the emitted light can in turn allow for discriminating between recognizers that emit light with different temporal characteristics. One example of a temporal characteristic is luminance lifetime. A fluorescent dye of a recognizer may emit photons in response to excitation. The probability of the luminescent molecule emitting a photon decreases with time after the excitation occurs. The rate of decay in the probability may be exponential. The “lifetime” is characteristic of how fast the probability decays over time. A fast decay is said to have a short lifetime, while a slow decay is said to have a long lifetime. Detecting temporal characteristics of the light emitted by dyes may allow distinguishing dyes that have different lifetimes. The photodetector described in the aforementioned U.S. Pat. No. 9,759,658 can detect the time of arrival of photons with nanosecond or picosecond resolution, and can time-bin the arrival of incident photons. Since the emission of photons is probabilistic, the label may be excited a plurality of times and any resulting photon emissions may be time-binned. Performing such a measurement a plurality of times allows populating a histogram of times at which photons arrived after an excitation event. This information can be analyzed to calculate a temporal characteristic of the emitted light.

As illustrated in the example of FIG. 1A, the sequencing data processing system 100 includes several modules that process sequencing data to generate AA variant data 116. In some embodiments, the sequencing data processing system 100 comprises one or more computer hardware processors configured to implement the modules of the sequencing data processing system 100. In some embodiments, the sequencing data processing system 100 may be separate from the sequencing device 120 (e.g., one or more computing devices separate from the sequencing device 120). In some embodiments, the sequencing data processing system 100 may be implemented on the sequencing device 120 (e.g., using one or more computer hardware processors and memory of the sequencing device 120). In some embodiments, one or more modules of the sequencing data processing system 100 may be implemented on the sequencing device 120 while one or more other modules of the sequencing data processing system 100 may be implemented using computing device(s) separate from the sequencing device 120.

In some embodiments, peptides sequenced by the sequencing device 120 may be prepared using strain-promoted alkyne-azide cycloaddition (SPAAC) click chemistry for conjugating azido-lysine modified peptides to linker molecules. During sequencing, aminopeptidases may sequentially cleave N-terminal amino acids from immobilized peptides, with the N-terminal amino acid cleavage time by aminopeptidases ranging from approximately 10-40 minutes.

As illustrated in the example of FIG. 1A, the sequencing data processing system 100 comprises a pulse identification module 102 that receives and processes raw sequencing data from the sequencing device 120. The pulse identification module 102 may be configured to analyze raw signal data to produce a set of pulse calls that can be fed into downstream signal processing (e.g., performed by a bioinformatics software application). The pulse identification module 102 may detect signals produced by binding events between fluorescently tagged NAA recognizers and peptides, and may distinguish binding event signals from background noise such as fluorescence from freely diffusing recognizers in a reaction chamber.

As illustrated in the example of FIG. 1A, the sequencing data processing system 100 further comprises a pulse segmentation module 104 connected to the pulse identification module 102. The pulse segmentation module 104 may be configured to segment identified pulses into recognition segments. Each recognition segment may indicate a particular time period in which a particular fluorescently tagged NAA recognizer or a group of fluorescently tagged NAA recognizers was binding to a particular NAA of a peptide being sequenced. In some embodiments, the pulse segmentation module 104 may be configured to identify temporal segments by detecting where pulsing patterns change over a duration of a sequencing run.

As illustrated in the example of FIG. 1A, the sequencing data processing system 100 comprises a recognizer assignment module 106. The recognizer assignment module 106 may be configured to assign fluorescently tagged NAA recognizers to recognition segments (e.g., by identifying dyes of the recognizers). The recognizer assignment module 106 includes a classification model 106A that the recognizer assignment module 106 may be configured to use to process fluorescence data to determine which recognizer corresponds to each recognition segment. For example, the classification model 106A may process fluorescence intensity and fluorescence decay information from recognition segments to identify which of a set of NAA recognizers (e.g., a set of six NAA recognizers) was binding during each recognition segment.

As illustrated in the example of FIG. 1A, the sequencing data processing system 100 comprises an alignment module 108 connected to the recognizer assignment module 106. The alignment module 108 may be configured to align reads to reference peptide sequences. The alignment module 108 may match each recognition segment in a read to an amino acid residue in a reference peptide sequence such that an expected recognizer for the amino acid residue matches a recognizer label of the recognition segment.

As illustrated in the example of FIG. 1A, the sequencing data processing system 100 comprises an AA variant detection module 110 connected to the alignment module 108. The AA variant detection module 110 may be configured to detect amino acid variants using alignments produced by the alignment module 108. For example, the AA variant detection module 110 may produce AA variant data 116 as output, which may contain information about detected amino acid variants including positions and quantification of the variants.

As illustrated in the example of FIG. 1A, the sequencing data processing system 100 comprises a reference alignment data generation module 112, which generates reference alignment data 114 (e.g., that may be used by the alignment module 108 and the AA variant detection module 110 for respective functions). The reference alignment data generation module 112 includes a machine learning model 112A trained to predict kinetic properties (e.g., pulse durations) for amino acid residue motifs. The machine learning model 112 allows for the generation of a comprehensive kinetic database that pairs amino acid sequences with corresponding expected kinetic properties (e.g., pulse duration values). The reference alignment data 114 may be accessed by other modules of the sequencing data processing system 100 (e.g., alignment module 108 and the AA variant detection module 110 to facilitate alignment scoring and variant detection operations).

FIG. 1B illustrates interaction between modules of the sequencing data processing 100 system of FIG. 1A in processing sequencing data generated by the sequencing device 120, according to some embodiments of the technology described herein. The sequencing data processing system 100 receives sequencing data 124 as input. The sequencing data 124 may be generated from detecting light emissions by fluorescently tagged NAA recognizers in response to illumination (e.g., laser illumination) during sequencing of peptide(s) in the peptide sample 122. In some embodiments, the sequencing data 124 may comprise raw time-intensity traces for individual time periods (e.g., time bins) after illumination (e.g., laser illumination). For example, the data may include a time-intensity trace for a first bin of a photodetector having photon counts over time detected by the first bin and a time-intensity trace for a second bin of a photodetector having photon counts over time detected by the second bin. In some embodiments, the sequencing data 124 may be computed by passing raw time-intensity traces through a function designed to reduce a size of the data.

As shown in the example of FIG. 1B, the sequencing data 124 is provided to the pulse identification module 102, which processes the sequencing data 124 to generate a light pulse trace 130. The pulse identification module 102 may be configured to generate multiple light pulse traces including the light pulse trace 130 (though only light pulse trace 130 is shown in FIG. 1B for illustrative purposes). The light pulse trace 130 may represent detected light emissions from fluorescently tagged NAA recognizers during peptide sequencing. The pulse identification module 102 may be configured to detect signals produced by binding events between recognizers and peptides, and may distinguish binding event signals from background noise.

In some embodiments, the pulse identification module 102 may be configured to perform pulse calling. In some embodiments, the pulse identification module 102 may operate on reaction chambers of the sequencing device 120 independently and may begin by estimating statistical properties of background noise. Once an estimate within acceptable error bounds is established, the pulse identification module 102 may process data frames in real-time. At each time point, the pulse identification module 102 may track whether a signal is attributed solely to the background noise or if it includes a pulse from a recognizer-NAA interaction. The pulse identification module 102 may be configured to identify a transition from background to pulse by performing an edge detection test to identify a significant signal shift compared to the background noise's statistical distribution. Similarly, the pulse identification module 102 may be configured to detect a shift from pulse back to background when recent frames of the signal match the background distribution. In some embodiments, the pulse identification module 102 may be configured to continuously update the background noise model in real time as new frames are observed (e.g., as new 60 ms frames are observed). In some embodiments, to account for the fact that detected pulses can represent either true recognizer-to-peptide interactions or transient noise spikes, a downstream filtering layer may evaluate the significance of pulses. This evaluation may consider factors such as pulse duration, intensity, and noise patterns across the duration of the run and the entire reaction chamber dataset.

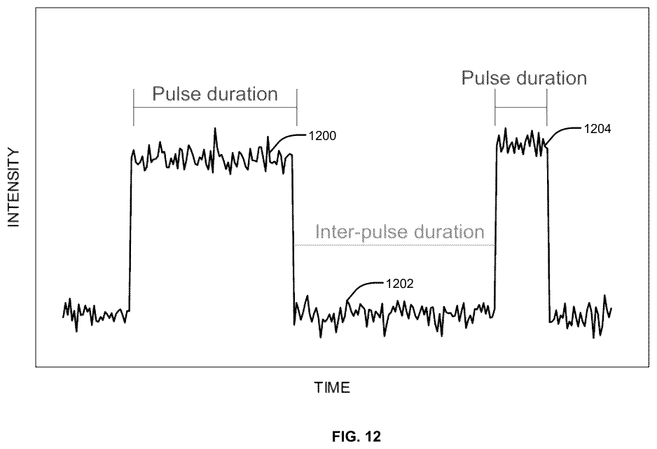

In some embodiments, each pulse identified by the pulse identification module 102 may represent a transient interaction between a recognizer and a peptide NAA. In some embodiment, the pulse identification module 102 may be configured to determine light pulse properties. The light pulse properties may include pulse durations (PDs) of light pulses and inter-pulse durations (IPDs) between successive light pulses. The pulse duration (PD) may be governed by dissociation kinetics of the recognizer-peptide complex. The inter-pulse duration (IPD) is the time from an end of one pulse and a beginning of a subsequent pulse. FIG. 12 illustrates example light pulse properties that may be determined by the pulse identification module 102, according to some embodiments of the technology described herein.

Referring to FIG. 12, a graph illustrates intensity as a function of time for a portion of a light pulse trace. The graph shows a first light pulse 1200 having a pulse duration indicated by a bracket spanning the width of the first light pulse 1200. The pulse duration of the first light pulse 1200 represents the time during which a fluorescently tagged NAA recognizer is bound to a particular NAA of a peptide being sequenced. The pulse duration may be governed by dissociation kinetics of the recognizer-peptide complex, as the duration of binding depends on how quickly the recognizer dissociates from the peptide.

With continued reference to FIG. 12, an inter-pulse period 1202 is shown as a region of lower intensity between pulses 1200 and 1204. The inter-pulse period 1202 represents the time between the end of the first light pulse 1200 and a beginning of the light pulse 1204. The inter-pulse duration, indicated by a bracket spanning the inter-pulse period 1202, may be governed by association kinetics of the recognizer-peptide complex. When a same recognizer-peptide interaction occurs in successive pulses, the inter-pulse duration is based on how quickly the recognizer re-associates with the peptide after dissociation. Both pulse duration and inter-pulse duration can be modeled using exponential distributions derived from theoretical first-order reaction kinetics. As further shown in FIG. 12, the third light pulse 1204 has a pulse duration indicated by a bracket spanning the width of the third light pulse 1204. The light pulses exhibit fluctuating intensity patterns during their respective pulse durations, while the inter-pulse period 1202 shows a baseline signal with smaller amplitude variations corresponding to background noise.

In some embodiments, the pulse identification module 102 may be configured to use an edge detection approach for identifying transitions from background to pulse. A transition from background to pulse may occur when an edge detection test identifies a significant signal shift compared to a statistical distribution of the background noise. Similarly, a transition from pulse back to background may occur when recent frames of the signal match the background distribution. In some embodiments, the pulse identification module 102 may be configured to continuously update a background noise model in real time as new frames are observed. For example, the pulse identification module 102 may update the background noise model as new 60 ms frames are observed during a sequencing run performed by the sequencing device 120.

As shown in the example of FIG. 1B, the light pulse trace 130 is provided to the pulse segmentation module 104, which may be configured to segment the light pulse trace 130 into a read 132. The pulse segmentation module 104 may be configured to segment the light pulse trace 130 into multiple portions called “recognition segments” that represent respective time periods of a recognizer binding to a particular NAA of a peptide. The read 132 may thus comprise a sequence of recognition segments that each indicate a particular time period in which one or more of the fluorescently tagged NAA recognizers were binding to a particular NAA of the at least one peptide. As illustrated in FIG. 1B, the read 132 includes a first recognition segment 132A, a second recognition segment 132B, and a third recognition segment 132C. It should be appreciated that the read 132 may include additional recognition segments in addition to those illustrated in FIG. 1B. In some embodiments, the pulse segmentation module 104 may be configured to identify the sequence of recognition segments by detecting where pulsing patterns change over a duration of a sequencing run. In some embodiments, the pulse segmentation module 104 may be configured to generate a plurality of reads using the light pulse durations and the inter-pulse durations, with each read comprising a sequence of recognition segments.

As illustrated in the example of FIG. 1B, the read 132 is provided to the recognizer assignment module 106, which includes the classification model 106A. In some embodiments, the recognizer assignment module 106 may be configured to assign fluorescently tagged NAA recognizers determined to be binding in the recognition segments to the recognition segments in the read 132. The classification model 106A may process fluorescence data from the recognition segments to identify which recognizer was binding during each recognition segment. As illustrated in FIG. 1B, the recognizer assignment module 106 assigns recognizer(s) 134A to the first recognition segment 132A, recognizer(s) 134B to the second recognition segment 132B, and recognizer(s) 134C to the third recognition segment 132C. The recognizer assignment module 106 may be configured to associate recognition segments 132A, 132B, 132B with respective recognizers 134A, 134B, 134C. In some embodiments, the recognizer assignment module 106 may be configured to select each of the recognizers 134A, 134B, 134C from a pre-determined set of recognizers (e.g., using the classification module 106A).

As illustrated in the example of FIG. 1B, the read 132 with assigned recognizers 134A, 134B, 134C is provided to the alignment module 108, which also accesses the reference alignment data 114. In some embodiments, the alignment module 108 may be configured to align the read 132 to one or more reference peptide sequences including reference peptide sequence 138 to produce one or more alignments including alignment 136. The alignment module 108 may be configured align a read to each of one or more reference peptide sequences to obtain one or more peptide alignments at least in part by using: (1) the fluorescently tagged NAA recognizers assigned to recognition segments of the at least one read, (2) light pulse durations in the recognition segments, and (3) inter-pulse durations. The alignment 136 may show correspondence between the recognition segments of the read 132 and amino acid residues in the reference peptide sequence 138.

As illustrated in the example of FIG. 1B, the alignment 136 is provided to the AA variant detection module 110, which may be configured to use the alignment 136 to detect one or more amino acid variants in peptide(s) of the peptide sample 122 using the peptide alignment(s) including peptide alignment 136. The AA variant detection module 110 outputs the AA variant data 116, which may contain information about detected amino acid variants including positions and quantification of the variants. For example, the AA variant data 116 may include a ratio of a quantity of a particular peptide variant to a quantity of one or more other peptide variants. As another example, the AA variant data 116 may include an identification of AA variants present in the peptide(s).

As illustrated in the example of FIG. 1B by the dashed lines around the alignment module 108, in some embodiments, the AA variant detection module 110 may be configured to perform amino acid variant detection without using alignment(s) from the alignment module 108. In such embodiments, the alignment module 108 may not be used to generate alignments (e.g., alignment 136) or the AA variant detection module 110 may not obtain alignments from the alignment module 108. In some embodiments, the AA variant detection module 110 may be configured to detect one or more amino acid variants in peptide(s) using the plurality of reads and an assignment of fluorescently tagged NAA recognizers to recognition segments of reads including the read 132. The AA variant detection module 110 may be configured to construct a multidimensional feature space by integrating aggregated positional kinetics. These inputs may capture recognizer read amino acid residue and, optionally, corresponding context variation. The AA variant detection module 110 may be configured to use the assignment of fluorescently tagged NAA recognizers to recognition segments to determine amino acid variant identities of the plurality of reads without requiring alignment to reference peptide sequences.

In some embodiments, the AA variant detection module 110 may be configured to determine amino acid variant identities of the plurality of reads using a trained machine learning model. The trained machine learning model may process features derived from the recognizer assignments and light pulse properties (e.g., pulse durations and/or inter-pulse durations) of the recognition segments to classify each read as corresponding to a particular amino acid variant. This enables variant discrimination in scenario where alignment is unavailable or otherwise difficult.

In some embodiments, the trained machine learning model may comprise a classification model, and the AA variant detection module 110 may be configured to train the classification model by clustering the plurality of reads to obtain multiple classes each corresponding to a particular amino acid variant. For example, a multi-component GMM (e.g., two-component GMM) may be trained using these features, with initial centroids guided by the expected kinetic profiles from the kinetic database and recognizer identities. Applying the trained GMM to the dataset yields amino acid variant identities for the reads. The AA variant detection module 110 may be configured to classify each read into one of the classes to obtain an amino acid variant identity of the read. In some embodiments, the clustering may be performed using dynamic time warping, k-means clustering, or other suitable clustering techniques that group reads based on similarity of recognizer assignments to recognition segments and light pulse properties of the recognition segments.

In some embodiments, the AA variant detection module 110 may be configured to cluster the plurality of reads based on light pulse properties extracted from the recognition segments. The AA variant detection module 110 may be configured to use pre-selection of primary features for analysis to enhance the accuracy of variant calling. The clustering may group reads into variant populations based on these discriminative features, enabling the AA variant detection module 110 to determine variant identities without requiring explicit alignment to reference sequences.

In some embodiments, the AA variant detection module 110 may be configured to determine amino acid variant identities for reads that share common recognizer assignments at variant positions. Mixtures of variants that share the same recognizer at the variant position pose challenges and sometimes diminish the discriminative power of recognizer-based clustering. Similarly, variants that are fully invisible under current conditions can be difficult to distinguish purely based on kinetics, particularly in extreme ratio scenarios. In such cases, the AA variant detection module 110 may leverage kinetic features at upstream positions to differentiate between variant populations. Even complex scenarios such as invisible-to-invisible variants may be resolved, underscoring the utility of the AA variant detection module 110 in extracting information from sparse data. The clustering-based approach may enable the AA variant detection module 110 to capture general trends and kinetic distinctions necessary for population differentiation across various types of single amino acid variants.

Referring to FIG. 1C, the reference alignment data generation module 112 and associated components are illustrated. The reference alignment data generation module 112 receives amino acid sequences 140 as input. The amino acid sequences 140 may comprise pentameric sequences such as RFNEL, FNELN, NELNF, and ELNFD. In some embodiments, some of the amino acid sequences 140 may have associated pulse duration times. For example, a pulse duration time 142A of 0.52 seconds may be associated with the RFNEL sequence, and a pulse duration time 142B of 0.37 seconds may be associated with the NELNF sequence. The pulse duration times 142A and 142B may represent empirically measured pulse durations obtained from sequencing runs in which the corresponding amino acid sequences were observed.

FIG. 1C illustrates generation of reference alignment data by reference alignment data generation module 112 of the sequencing data processing system of FIG. 1A, according to some embodiments of the technology described herein. The reference alignment data generation module 112 includes the machine learning model 112A that processes the amino acid motifs 140 and corresponding pulse duration times (e.g., pulse duration times 142A, 142B) to generate pulse duration times for various. The reference alignment data generation module 112 may be configured to train the machine learning model 112A using amino acid motifs for which there are empirically determined pulse duration times (e.g., the amino acid motifs corresponding to pulse duration times 142A, 142B). The machine learning model 112A may be configured to predict pulse durations for amino acid motifs. The reference alignment data generation module 112 outputs the reference alignment data 114, which may be stored as a reference dataset. in some embodiments, the reference alignment data 114 may be stored in any suitable storage structure. For example, the reference alignment data 114 may be stored in a database structure. The reference alignment data 114 may contain amino acid motifs paired with corresponding pulse duration values. For example, the reference alignment data 114 may include RFNEL paired with 0.52 seconds, FNELN paired with 4.27 seconds, NELNF paired with 0.37 seconds, and ELNFD paired with 0.40 seconds.

In some embodiments, the amino acid motifs of the reference alignment data 114 may be 2, 3, 4, 5, 6, 7, 8, 9, 10, 11, 12, 13, 14, or 15 amino acid residues in length. For example, the amino acid motifs may be 6 amino acid residues in length. As another example, the amino acid motifs may be 5 amino acid residues in length (e.g., as illustrated in FIG. 1C). In some embodiments, all the amino acid motifs in the reference alignment data 114 may be of the same length. In some embodiments, the amino acid motifs in the reference alignment data 114 may be of different lengths. In some embodiments, the reference alignment data 114 may include empirical pulse duration values when previously measured for a corresponding amino acid motif, and may include predicted values otherwise. For example, when a pulse duration has been previously measured for a particular pentameric motif during sequencing runs, the empirical value may be stored in the reference alignment data 114. When a pulse duration has not been previously measured for a particular amino acid motif, the machine learning model 112A may be used to predict the pulse duration, and the predicted value may be stored in the reference alignment data 114. This approach allows the reference alignment data generation module 112 to generate a comprehensive kinetic database that pairs all possible amino acid motifs of a particular length with corresponding pulse duration values for use in downstream alignment and/or variant detection processes.

In some embodiments, the alignment module 108 may be configured to access the reference alignment data 114 as a reference dataset storing pulse durations for amino acid residue sequences. The alignment module 108 may be configured to determine, using the pulse durations for the amino acid motifs stored in the reference alignment data 114, expected light pulse durations for amino acid residues in a reference peptide sequence. For each of a set of target amino acid residues in the reference peptide sequence that are aligned with recognition segments of a read (e.g., the shaded amino acid residues in reference peptide sequence 138 shown in FIG. 1B), the alignment module 108 may be configured to identify a subsequence of the reference peptide sequence composed of the target amino acid residue and a particular number of amino acid residues ((e.g., 1, 2, 3, 4, 5, 6, 7, 8, 9, 10, or other suitable number of amino acid residues) preceding the amino acid residue. The alignment module 108 may be configured to identify, in the reference alignment data 114, one of the amino acid motifs that matches the subsequence. The alignment module 108 may be configured to determine, as an expected pulse duration for the target amino acid residue, a pulse duration stored for the identified amino acid motif in the reference alignment data 114 (e.g., in performing alignment).

In some embodiments, at least some of the pulse durations stored in the reference alignment data 114 for at least some of the amino acid motifs may be determined using the machine learning model 112A trained to predict pulse durations. The machine learning model 112A may be trained using empirical data from sequencing runs to learn relationships between amino acid motifs and pulse duration measurements. The trained machine learning model 112A may then be applied to predict pulse durations for amino acid residue motifs that have not been empirically measured, allowing the reference alignment data 114 to contain pulse duration values for a comprehensive set of amino acid motifs.

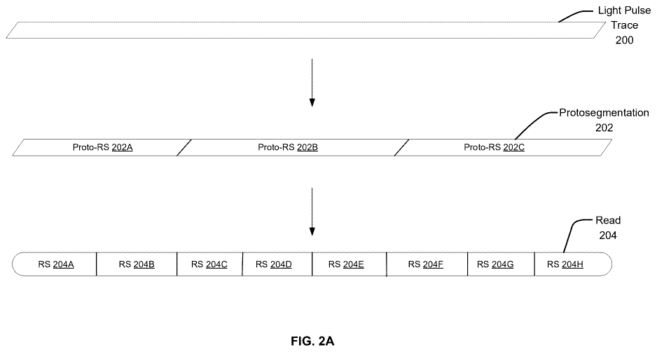

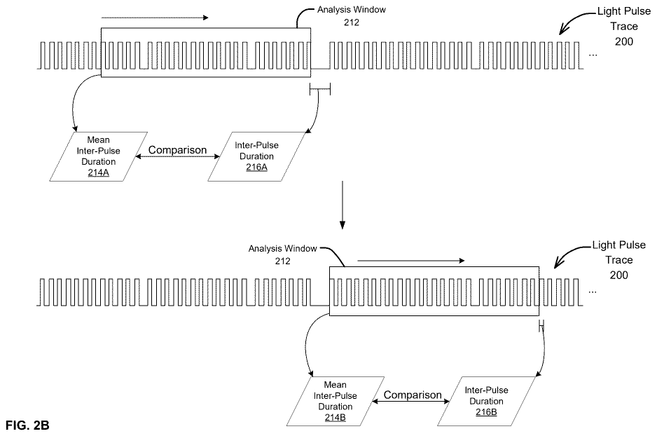

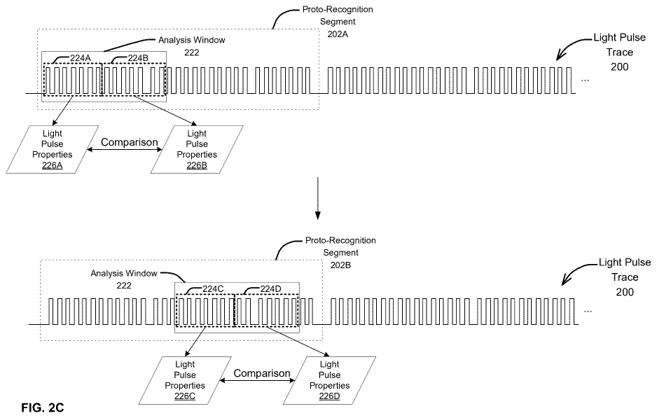

FIG. 2A illustrate example generation of a read by segmentation of a light pulse trace into recognition segments that form the read, according to some embodiments of the technology described herein. Referring to FIG. 2A, the diagram illustrates segmentation of a light pulse trace 200 into recognition segments through a two-stage process performed by the pulse segmentation module 104 of the sequencing data processing system 100 described herein with reference to FIGS. 1A-1C. The light pulse trace 200 is shown at the top of FIG. 2A as an elongated bar representing raw sequencing data that has been processed by the pulse identification module 102. The light pulse trace 200 may contain identified light pulses (e.g., by performing pulse calling) generated from binding events between fluorescently tagged NAA recognizers and peptides during sequencing by the sequencing device 120.

As illustrated in FIG. 2A, the light pulse trace 200 undergoes a proto-segmentation 202 stage. During the proto-segmentation 202, the pulse segmentation module 104 may be configured to divide the light pulse trace 200 into multiple proto-recognition segments. In the example of FIG. 2A, the proto-segmentation 202 produces three proto-recognition segments for a portion of the light pulse trace 200: a proto-recognition segment 202A, a proto-recognition segment 202B, and a proto-recognition segment 202C. Each proto-recognition segment 202A, 202B, 202C is shown as a separate horizontal bar segment. The proto-recognition segments 202A, 202B, 202C may represent regions of active pulsing that do not contain large gaps in time between pulses. The pulse segmentation module 104 may identify boundaries between the proto-recognition segments 202A, 202B, 202C by detecting where regions of active pulsing terminate, such as where a peptide NAA state transitions from a residue that is detectable by a recognizer to one that is not detectable.

With continued reference to FIG. 2A, in some embodiments, the pulse segmentation module 104 may be configured to divide the proto-recognition segments 202A, 202B, 202C to generate a read 204. The read 204 represents a final segmented output produced by the pulse segmentation module 104. As illustrated in FIG. 2A, the read 204 includes the following recognition segments: a recognition segment 204A, a recognition segment 204B, a recognition segment 204C, a recognition segment 204D, a recognition segment 204E, a recognition segment 204F, a recognition segment 204G, and a recognition segment 204H. Each recognition segment 204A, 204B, 204C, 204D, 204E, 204F, 204G, 204H is depicted as a distinct compartment within the horizontal bar of the read 204. The subdivision of the proto-recognition segments 202A, 202B, 202C into the recognition segments 204A, 204B, 204C, 204D, 204E, 204F, 204G, 204H may be performed by the pulse segmentation module 104 to identify individual time periods corresponding to specific binding interactions between fluorescently tagged NAA recognizers and particular NAAs of a peptide being sequenced.

In some embodiments, each recognition segment 204A, 204B, 204C, 204D, 204E, 204F, 204G, 204H in the read 204 may indicate a particular time period in which one or more fluorescently tagged NAA recognizers were binding to a particular NAA of the peptide. The fluorescently tagged recognizer(s) may consist of a particular fluorescently tagged NAA recognizer or a particular group of fluorescently tagged recognizer(s). For example, a particular group of fluorescently tagged recognizers may be recognizers of a common type. Recognizers of the common type may, for example, switch out with one another during a recognition segment. As another example, a particular group of fluorescently tagged recognizers may be recognizers of different types that belong to a common class. Recognizers of different types that belong to a common class may switch out with one another during a recognition segment. The pulse segmentation module 104 may be configured to subdivide the proto-recognition segments 202A, 202B, 202C by detecting where pulsing properties change within each proto-recognition segment, indicating a change in the recognizer-NAA interaction. For example, the proto-recognition segment 202A may be subdivided into the recognition segments 204A, 204B, 204C when the pulse segmentation module 104 detects changes in light pulse properties such as pulse duration, inter-pulse duration, fluorescence intensity, and/or fluorescence decay that indicate different recognizer-NAA binding interactions occurred during different portions of the proto-recognition segment 202A. Similarly, the proto-recognition segment 202B may be subdivided into the recognition segments 204D, 204E, 204F, and the proto-recognition segment 202C may be subdivided into the recognition segments 204G, 204H. The read 204 may then be provided to the recognizer assignment module 106 for assignment of fluorescently tagged NAA recognizers to each of the recognition segments 204A, 204B, 204C, 204D, 204E, 204F, 204G, 204H.

FIG. 2B illustrates example segmentation of the light pulse trace into proto-recognition segments, according to some embodiments of the technology described herein. Referring to FIG. 2B, a diagram illustrates trace segmentation for identifying proto-recognition segments in the light pulse trace 200. The pulse segmentation module 104 may be configured to identify boundaries where regions of active pulsing terminate by scanning an analysis window 212 across the light pulse trace 200. In some embodiments, the analysis window 212 may be a sliding window that encompasses a particular number of light pulses. For example, the analysis window 212 may be a sliding window of pulses in one of the following ranges: 1-10 pulses, 10-20 pulses, 20-30 pulses, 30-40 pulses, 40-50 pulses, 50-60 pulses, or another suitable range. For example, the analysis window 212 may be a 30-pulse window that scans across the light pulse trace 200.

In an upper portion of FIG. 2B, the analysis window 212 spans a section of the light pulse trace 200, which shows a series of vertical lines representing light pulses with varying spacing. The pulse segmentation module 104 may be configured to determine a mean inter-pulse duration 214A of pulses within the analysis window 212. The mean inter-pulse duration 214A may be calculated as an average of inter-pulse durations between successive light pulses within the analysis window 212. The pulse segmentation module 104 may be configured to determine an inter-pulse duration 216A measured between a final pulse of the analysis window 212 and a subsequent pulse that follows the analysis window 212. The pulse segmentation module 104 may be configured to compare the mean inter-pulse duration 214A to the inter-pulse duration 216A to determine whether a boundary exists at the position of the analysis window 212.

With continued reference to FIG. 2B, a lower portion of the figure shows the light pulse trace 200 at a later stage of analysis. The analysis window 212 has advanced along the light pulse trace 200 to a different position. At the repositioned analysis window 212, the pulse segmentation module 104 may be configured to determine a mean inter-pulse duration 214B calculated from pulses within the repositioned analysis window 212. The pulse segmentation module 104 may be configured to determine an inter-pulse duration 216B measured between a final pulse of the repositioned analysis window 212 and a subsequent pulse. The pulse segmentation module 104 may be configured to compare the mean inter-pulse duration 214B to the inter-pulse duration 216B to identify whether a boundary exists at the repositioned analysis window 212.

In some embodiments, the pulse segmentation module 104 may be configured to divide the light pulses into proto-recognition segments based on a result of comparing the mean inter-pulse durations of the light pulse windows to the inter-pulse durations between the final light pulses of the light pulse windows and the respective subsequent light pulses. The pulse segmentation module 104 may be configured to determine that a gap is significant when the gap is greater than a threshold multiple of the mean inter-pulse duration of the preceding analysis window 212. In some embodiments, the pulse segmentation module 104 may determine that a gap is significant when the inter-pulse duration 216A is greater than one of the following: 2, 3, 4, 5, 6, 7, 8, 9, 10, 11, 12, 13, 14, 15, 16, 17, 18, 19, 20, 21, 22, 23, 24, or 25 times the mean inter-pulse duration 214A. For example, the pulse segmentation module 104 may determine that a gap is significant when the inter-pulse duration 216A is greater than 12 times the meal inter-pulse duration 214A. When the inter-pulse duration 216A exceeds the threshold, the pulse segmentation module 104 may make a split at that position in the light pulse trace 200. The rationale for this threshold is that, given an exponential model of inter-pulse durations in the preceding region, a high percentage of inter-pulse durations (e.g., 99.9999%) may be less than 12 times the mean, and thus any gap greater than 12 times the mean inter-pulse duration may indicate an end of the recognizer-NAA binding process of the preceding pulses.

In some embodiments, the pulse segmentation module 104 may be configured to scan the light pulse trace 200 in both forward and reverse directions to identify boundaries where regions of active pulsing terminate. By scanning the trace in both directions, the pulse segmentation module 104 may identify boundaries that might be missed when scanning in only one direction. The pulse segmentation module 104 may be configured to divide the light pulse trace 200 into proto-recognition segments by splitting the trace at positions where significant gaps are detected during scanning in both directions. The proto-recognition segments may represent regions of active pulsing that do not contain large gaps in time between pulses. The pulse segmentation module 104 may then be configured to divide the proto-recognition segments to obtain the sequence of recognition segments that form a read.

FIG. 2C illustrates example subdivision of proto-recognition segments into recognition segments by the pulse segmentation module 104, according to some embodiments of the technology described herein. Referring to FIG. 2C, a diagram illustrates a comparison of light pulse properties within an analysis window 222 applied to the light pulse trace 200 at two different positions corresponding to the proto-recognition segment 202A and the proto-recognition segment 202B. The pulse segmentation module 104 may be configured to divide the proto-recognition segments to obtain the sequence of recognition segments by comparing sequential pairs of light pulse windows in each proto-recognition segment and dividing the proto-recognition segment into multiple recognition segments based on a result of comparing the sequential pairs of light pulse windows.

In an upper portion of FIG. 2C, the analysis window 222 is positioned over a portion of the proto-recognition segment 202A of the light pulse trace 200. The analysis window 222 encompasses a first light pulse window 224A and a second light pulse window 224B. In some embodiments, the analysis window 222 may be a sliding window that encompasses a particular number of light pulses. For example, the analysis window 222 may be a sliding window of pulses in one of the following ranges: 40-50 pulses, 50-60 pulses, 60-70 pulses, 70-80 pulses, or another suitable range. For example, the pulse segmentation module 104 may use a 60-pulse sliding window to subdivide proto-recognition segments into recognition segments. In some embodiments, the first light pulse window 224A may encompass a first half of the pulses in the analysis window 222 (e.g., the first 30 pulses of a 60-pulse window), and the second light pulse window 224B may encompass a second half of the pulses in the analysis window 222 (e.g., the last 30 pulses of a 60-pulse window).

With continued reference to FIG. 2C, light pulse properties 226A are extracted from the first light pulse window 224A, and light pulse properties 226B are extracted from the second light pulse window 224B. The pulse segmentation module 104 may be configured to compare a first measurement of one or more light pulse properties in the first light pulse window 224A to a second measurement of the one or more light pulse properties in the second light pulse window 224B. The light pulse properties 226A and the light pulse properties 226B may each comprise one or more of light pulse duration, inter-pulse duration, fluorescence intensity, and fluorescence decay. In some embodiments, the pulse segmentation module 104 may be configured to apply a statistical test on each light pulse property using a result of comparing the first measurement to the second measurement to obtain output indicating a probability that the first light pulse window 224A and the second light pulse window 224B correspond to a common binding interaction between one or more of the fluorescently tagged NAA recognizers and a particular NAA of the at least one peptide.

In some embodiments, the pulse segmentation module 104 may be configured to use any suitable statistical test to compare a light pulse property between the light pulse windows 224A, 224B. For example, the pulse segmentation module 104 may use a Kolmogorov-Smirnov (KS) test. The pulse segmentation module 104 may be configured to compute p-values for independent 2-sample KS tests, with one test for each light pulse property. The null hypothesis for each KS test may be that the pulses in the first light pulse window 224A have the same distribution of the given property as pulses in the second light pulse window 224B. In some embodiments, the pulse segmentation module 104 may compute p-values for four independent KS tests corresponding to pulse duration, inter-pulse duration, fluorescence intensity, and fluorescence decay. The pulse segmentation module 104 may record a minimum of the p-values for each potential split point across the proto-recognition segment 202A.

As further shown in FIG. 2C, an arrow indicates progression to a lower portion of the figure, where the analysis window 222 has shifted along the light pulse trace 200 to a position corresponding to the proto-recognition segment 202B. At this position, the analysis window 222 encompasses a first light pulse window 224C and a second light pulse window 224D. Light pulse properties 226C are extracted from the first light pulse window 224C, and light pulse properties 226D are extracted from the second light pulse window 224D. The pulse segmentation module 104 may be configured to compare the light pulse properties 226C to the light pulse properties 226D using the same statistical testing approach described above for the comparison between pulse window 224A and pulse window 224B.

In some embodiments, the pulse segmentation module 104 may be configured to divide the proto-recognition segment into multiple recognition segments using outputs obtained from statistical tests applied on the one or more light pulse properties for the sequential pairs of light pulse windows. In some embodiments, the pulse segmentation module 104 may be configured to use a particular p-value threshold to determine whether to split a proto-recognition segment. For example, the pulse segmentation module 104 may use a p-value threshold of 10⁻⁴ for the KS test to determine whether to split a proto-recognition segment. When a minimum p-value across all potential split points is less than 10⁻⁴, the pulse segmentation module 104 may split the proto-recognition segment at that pulse index. In some embodiments, the pulse segmentation module 104 may be configured to repeat the p-value computations and splitting process on the resulting segments and on any segments emanating from splitting those segments, until no p-value meets the threshold for further splitting. The resulting set of segments may be the recognition segments that form the read 204.

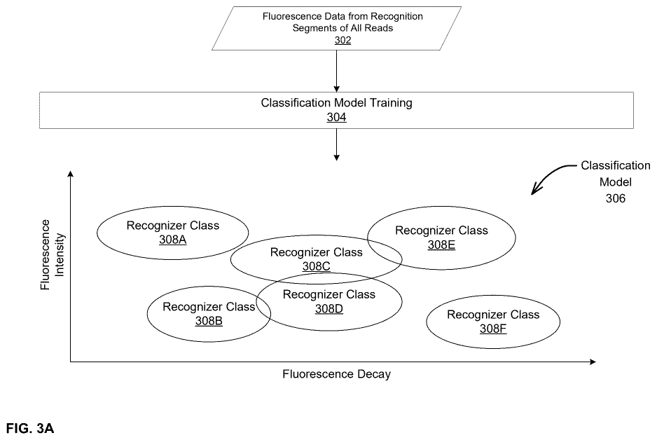

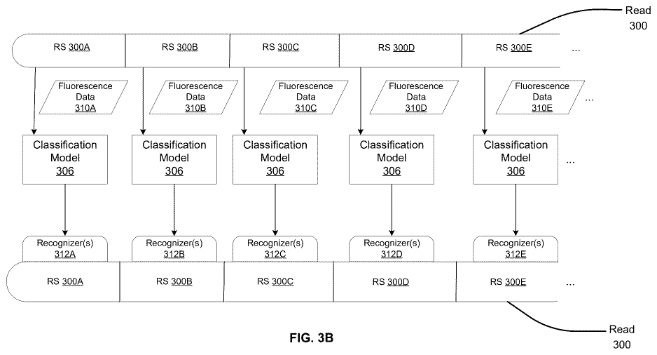

FIG. 3A illustrates an example training of a recognizer classification model using fluorescence data obtained from reads, according to some embodiments of the technology described herein. Referring to FIG. 3A, a diagram illustrates a classification model training 304 process using fluorescence data 302 from recognition segments of reads to generate a classification model 306. The fluorescence data 302 may be obtained from recognition segments of all reads generated by the pulse segmentation module 104 from sequencing data obtained for a sample (e.g., peptide sample 122). The fluorescence data 302 may comprise fluorescence data for light pulses emitted by the fluorescently tagged NAA recognizers during sequencing of peptide(s) in the peptide sample 122. In some embodiments, obtaining the fluorescence data 302 for the light pulses may comprise, for each of multiple ones of the light pulses, obtaining a log of fluorescence intensity detected after illumination that caused emission of the light pulse. In some embodiments, obtaining the fluorescence data 302 for the light pulses may comprise, for each of multiple ones of the light pulses, obtaining a ratio between: (1) a number of photons detected in a first time bin after the illumination, and (2) a number of photons detected in a second time bin after the illumination, wherein the second time bin is subsequent to the first time bin.

With continued reference to FIG. 3A, the fluorescence data 302 is provided as input to the classification model training 304, which produces the classification model 306 as output. The recognizer assignment module 106 may be configured to train any suitable classification model. For example, the classification model 306 may comprise a Gaussian Mixture Model (GMM) classifier (e.g., as illustrated in FIG. 3A). The classification model 306 may be trained on-the-fly using pulses sampled from across a chip of the sequencing device 120 and throughout a sequencing run. The classification model training 304 may train the classification model 306 using the fluorescence data 302 obtained for the light pulses emitted by the fluorescently tagged NAA recognizers during sequencing. In some embodiments, the classification model 306 may comprise clusters obtained from clustering the fluorescence data 302. In

Although in the example of FIG. 3A the classification model 306 is produced by applying a training algorithm to sequencing data, in some embodiments, the classification model 306 may be a pre-trained model. For example, the classification model 306 may be trained using fluorescence data obtained from previously performed sequencing of one or more samples. In some embodiments, the classification model 306 may be trained using an unsupervised learning technique (e.g., clustering). In some embodiments, the classification model 106A may be performed using a supervised learning technique (e.g., stochastic gradient descent). A supervised learning algorithm may be applied to a labeled training dataset comprising sets of fluorescence data and corresponding known recognizers (e.g., determined empirically). In some embodiments, the classification model 306 may be any suitable machine learning model. For example, the classification model 306 may be a GMM classifier, a K-means clustering model, a support vector machine (SVM) classifier, a K-nearest neighbors (KNN) classifier, a random forest classifier, a logistic regression classifier, a neural network classifier, a Naïve Bayes classifier, a decision tree classifier, a gradient boosting classifier, a linear discriminant analysis (LDA) classifier, a quadratic discriminant analysis (QDA) classifier, or another suitable classifier.

As shown in the example of FIG. 3A, the classification model 306 is represented as a two-dimensional plot with fluorescence intensity on a vertical axis and fluorescence decay on a horizontal axis. In some embodiments, the classification model 306 may use a bin ratio as a proxy for fluorescence decay lifetime. The bin ratio may be a ratio of measurements from two collection time periods (e.g., bins) with different delays following laser illumination. For example, the sequencing device 120 may measure fluorescence decay lifetime by sampling in two time periods beginning at different delays following laser illumination, and the ratio of these two measurements (the bin ratio) may be used as a proxy for the fluorescence decay lifetime in downstream data analysis performed by the recognizer assignment module 106.

As shown in the example of FIG. 3A, within the classification model 306, six recognizer classes are distributed across the plot. A first recognizer class 308A is positioned in an upper left region of the plot. A second recognizer class 308B is positioned below and to the right of the first recognizer class 308A. A third recognizer class 308C is positioned in a central region of the plot. A fourth recognizer class 308D is positioned below the third recognizer class 308C. A fifth recognizer class 308E is positioned to the right of the third recognizer class 308C. A sixth recognizer class 308F is positioned in a lower right region of the plot. Each of the recognizer classes 308A, 308B, 308C, 308D, 308E, and 308F is depicted as an elliptical region representing a cluster of fluorescence data corresponding to a respective fluorescently tagged NAA recognizer. In some embodiments, each of the recognizer classes may indicate a particular dye associated with a particular recognizer. In some embodiments, each ellipse may represent one standard deviation of a normal distribution associated with each dye in the GMM classifier. After the GMM is fit during the classification model training 304, each cluster may be associated with one of the dyes in a sequencing kit based on pre-calculated cross-run averages of measured intensities and bin ratios for each dye.

FIG. 3B illustrates assigning recognizers to recognition segments of a read 300 using the trained classification model 306, according to some embodiments of the technology described herein. Referring to FIG. 3B, the read 300 comprises multiple recognition segments including a recognition segment 300A, a recognition segment 300B, a recognition segment 300C, a recognition segment 300D, and a recognition segment 300E. Additional recognition segments may be present in the read 300 as indicated by ellipses in FIG. 3B. In some embodiments, the recognizer assignment module 106 may be configured to assign, to recognition segments in a plurality of reads including the read 300, fluorescently tagged NAA recognizers determined to be binding in the recognition segments. For each of the recognition segments 300A, 300B, 300C, 300D, 300E, the recognizer assignment module 106 may be configured to obtain fluorescence data for the recognition segment. The fluorescence data may indicate a detected fluorescence intensity and fluorescence decay of a fluorescent dye of fluorescently tagged NAA recognizer(s) binding in the recognition segment.

As illustrated in FIG. 3B, fluorescence data 310A is associated with the recognition segment 300A, fluorescence data 310B is associated with the recognition segment 300B, fluorescence data 310C is associated with the recognition segment 300C, fluorescence data 310D is associated with the recognition segment 300D, and fluorescence data 310E is associated with the recognition segment 300E. Each of the fluorescence data 310A, fluorescence data 310B, fluorescence data 310C, fluorescence data 310D, and fluorescence data 310E may comprise fluorescence intensity and fluorescence decay measurements for light pulses within the corresponding recognition segment.

In some embodiments, the recognizer assignment module 106 may be configured to identify, using the fluorescence data for each recognition segment, fluorescently tagged NAA recognizer(s) from among a set of candidate fluorescently tagged NAA recognizers for assignment to the recognition segment. The recognizer assignment module 106 may be configured to use the classification model 306 to process the fluorescence data to determine a classification of the fluorescence data as corresponding to particular fluorescently tagged NAA recognizer(s). As illustrated in FIG. 3B, the recognizer assignment module 106 provides fluorescence data 310A as input to the classification model 306 to obtain recognizer(s) 312A for assignment to the recognition segment 300A. Similarly, the recognizer assignment module 106 processes the fluorescence data 310B to identify recognizer(s) 312B for the recognition segment 300B, processes the fluorescence data 310C to identify recognizer(s) 312C for the recognition segment 300C, processes the fluorescence data 310D to identify recognizer(s) 312D for the recognition segment 300D, and processes the fluorescence data 310E to identify recognizer(s) 312E for the recognition segment 300E. In some embodiments, the recognizer assignment module 106 may be configured to determine a measure of similarity between the fluorescence data and each of multiple classes of the classification model 306. The multiple classes may each correspond to a respective one of the candidate fluorescently tagged NAA recognizers. The recognizer assignment module 106 may be configured to select, as the classification, using similarity measurements determined for the multiple classes, one of the multiple classes corresponding to a particular fluorescently tagged NAA recognizer or a particular group of fluorescently tagged NAA recognizers (e.g., recognizers of a common type or recognizers belonging to a common class).

In some embodiments, the recognizer assignment module 106 may be configured to calculate a dye purity for each recognition segment to determine which recognizer is associated with the recognition segment. The dye purity may measure how consistent a distribution of fluorescence intensities and bin ratios within the recognition segment are with each of the dyes associated with the classes in the classification model 306. For example, the recognizer assignment module 106 may be configured to calculate the dye purity using an exponential function based on a Mahalanobis distance between pulse measurements and a center of a cluster in the classification model 306. For a recognition segment, the dye purity Pᵢ for the i-th dye may be calculated as:

$${P}_{i} = \frac{2}{N} {\sum }_{n = 1}^{N} exp ( - \frac{1}{2} {( {x}_{n} - {μ}_{i} )}^{T} {\sum }_{i}^{- 1} ( {x}_{n} - {μ}_{i} ) )$$

where N is a number of pulses in the recognition segment, μᵢ is a center of the i-th dye's normal distribution in the classification model 306, Σᵢ is a covariance matrix of the i-th dye's normal distribution, and xₙ is a position in log-intensity and bin ratio space for the n-th pulse in the recognition segment. Purity values close to 1 may indicate a high level of agreement between what is predicted for a particular dye and what is measured within the recognition segment.

In some embodiments, the recognizer assignment module 106 may be configured to calculate expected dye purity values to predict what observed purity should be for all dyes given a hypothetical recognition segment consisting of pulses from a single dye. The expected dye purity values may be represented as a matrix Cᵢⱼ, where Cᵢⱼ is the purity of dye j that would be expected to observe for a hypothetical recognition segment consisting of pulses whose bin ratio and intensity are sampled from the distribution associated with dye i. The expected purity matrix Cᵢⱼ may be calculated as:

$${C}_{i j} = \frac{2 \cdot exp ( - \frac{1}{2} {( {μ}_{i} - {μ}_{j} )}^{T} {\sum }_{i}^{- 1} {( {\sum }_{i}^{- 1} + {\sum }_{j}^{- 1} )}^{- 1} {\sum }_{j}^{- 1} ( {μ}_{i} - {μ}_{j} ) )}{\sqrt{\vert {\sum }_{i} \vert \vert {\sum }_{i}^{- 1} + {\sum }_{j}^{- 1} \vert}}$$

In some embodiments, the recognizer assignment module 106 may be configured to determine a fluorescence dye composition distance between the fluorescence data and each of the multiple classes to obtain fluorescence dye composition distances as similarity measurements. To determine which recognizer to assign to each recognition segment, the recognizer assignment module 106 may be configured to calculate and normalize a purity vector P of the recognition segment and each column of the expected purity matrix C from the classification model 306. The recognizer assignment module 106 may be configured to calculate a distance between the purity vector P and each column of the expected purity matrix C. This distance may be referred to as the dye composition distance. The recognizer assignment module 106 may be configured to label each recognition segment with the recognizer that has a shortest dye composition distance.

As shown in the example of FIG. 3B, in some embodiments, the recognizer assignments are associated back with respective recognition segments in the read 300. The recognition segment 300A is assigned recognizer(s) 312A, the recognition segment 300B is assigned recognizer(s) 312B, the recognition segment 300C is assigned recognizer(s) 312C, the recognition segment 300D is assigned recognizer(s) 312D, and the recognition segment 300E is assigned recognizer(s) 312E. The ellipses in FIG. 3B indicate that this process continues for additional recognition segments in the read 300. The classification model 306 may be applied uniformly across all recognition segments to determine the fluorescently tagged NAA recognizers that were binding during each time period represented by the recognition segments.

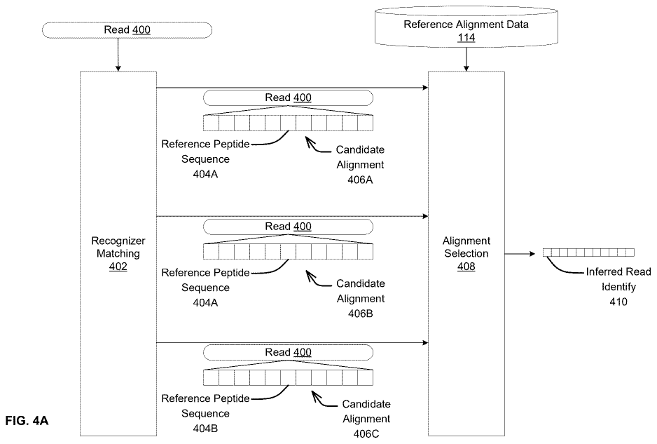

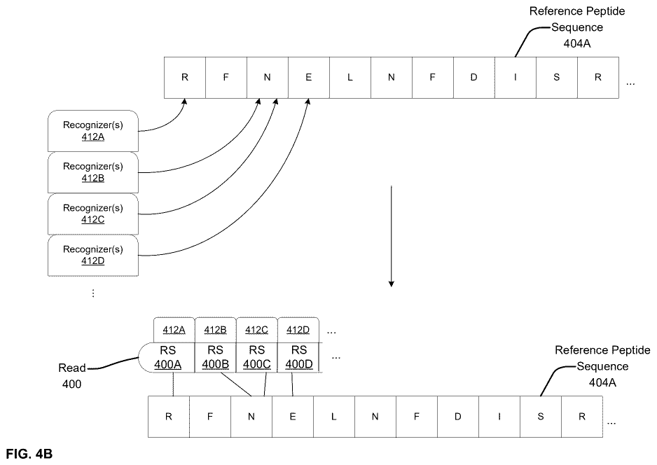

FIG. 4A illustrates example alignment of a read to reference peptide sequences, according to some embodiments of the technology described herein. Referring to FIG. 4A, the alignment module 108 may be configured to receive a read 400 as input and process the read 400 through a recognizer matching 402 operation to generate candidate alignments against reference peptide sequences stored in the reference alignment data 114. The recognizer matching 402 may be configured to compare the read 400 against multiple reference peptide sequences, including a reference peptide sequence 404A and a reference peptide sequence 404B. In some embodiments, the alignment module 108 may be configured to access, for amino acid residues in the reference peptide sequence 404A and the reference peptide sequence 404B, expected fluorescently tagged NAA recognizers. The alignment module 108 may be configured to assign recognition segments in the read 400 to amino acid residues in the reference peptide sequence 404A and the reference peptide sequence 404B at least in part by matching fluorescently tagged NAA recognizers assigned to the recognition segments to expected fluorescently tagged NAA recognizers of the amino acid residues in the reference peptide sequences 404A, 404B.

As illustrated in the example of FIG. 4A, in some embodiments, the recognizer matching 402 may be configured to generate multiple candidate alignments with the reference peptide sequences 404A, 404B. The comparison of the read 400 with the reference peptide sequence 404A may produce a candidate alignment 406A and a candidate alignment 406B, representing different possible alignments of the read 400 with the same reference peptide sequence 404A. The comparison of the read 400 with the reference peptide sequence 404B may produce a candidate alignment 406C. Each of the candidate alignments 406A, 406B, 406C shows the read 400 positioned relative to a corresponding reference peptide sequence, with the alignment indicating how recognition segments in the read 400 correspond to amino acid residues in the reference peptide sequence.

With continued reference to FIG. 4A, the candidate alignments 406A, 406B, and 406C are provided to an alignment selection 408 operation. The alignment selection 408 may be configured to evaluate the candidate alignments 406A, 406B, 406C using the reference alignment data 114 to determine an appropriate alignment. The alignment selection 408 may be configured to determine alignment scores for the candidate alignments 406A, 406B, 406C. In some embodiments, the alignment module 108 may be configured to select, using the alignment scores determined for the candidate alignments 406A, 406B, 406C, one of the candidate alignments as an alignment of the read 400 with a reference peptide sequence. The alignment selection 408 produces an inferred read identity 410 as output, which may represent a determined peptide identity (e.g., variant) of the read 400 based on a selected alignment.

In some embodiments, the alignment module 108 may be configured to determine alignment scores for the candidate alignments 406A, 406B, 406C by calculating a match score component. The match score s_{match} for a given pair of a recognition segment and a visible reference state may be calculated as:

$${s}_{match} = {w}_{match} \cdot exp ( - {( \frac{Δ log ( pd )}{{σ}_{match}} )}^{2} )$$

where Δ log (pd) is a difference between a log observed pulse duration and a log expected pulse duration, σ_{match} is a scaling parameter for a penalty, and w_{match} is a match score weight. In some embodiments, the alignment module 108 may use a match score weight (w_{match}) of 1 and a scaling parameter (σ_{match}) of 1 for calculating match scores. The reference alignment data 114 may provide expected pulse duration information and other alignment parameters used by both the recognizer matching 402 and the alignment selection 408 to score and evaluate the candidate alignments 406A, 406B, 406C. In some embodiments, the alignment module 103 may be configured to adjust the match score using a deletion score and/or a gap score described below.

In some embodiments, the alignment module 108 may be configured to permit deletions in alignments, with states that have higher expected pulse durations receiving larger deletion penalties. The deletion score may be designed this way because some very short pulse duration states may be missed due to a finite sampling rate of the sequencing device 120. In some embodiments, the alignment module 108 may be configured to calculate a deletion score s_{deletion} for a visible state that does not align to any recognition segment in a read using the following formula:

$${s}_{deletion} = - {w}_{deletion} \cdot max ( 0 , min ( 1 , 1.5 \times ( pd - 0.18 ) ) )$$

where pd is an expected pulse duration in seconds of the state and w_{deletion} is a deletion score weight. In some embodiments, the deletion score weight w_{deletion} may be set to 1. In some embodiments, because a number of amino acids sequenced may vary from read to read, the alignment module 108 may be configured to not apply a deletion penalty for any visible state following a state aligned to a final recognition segment in the read.

In some embodiments, the alignment module 108 may be configured to allow insertions only if a recognizer of an inserted recognition segment matches that of a recognition segment immediately preceding or following it in the read. The alignment module 108 may be configured to permit multiple adjacent recognition segments to align to a same state of a reference peptide sequence to ensure that a recognition segment that was erroneously split into multiple recognition segments can still align. In some embodiments, recognition segments that are determined to be low quality may be removed from a read prior to alignment. For example, recognition segments with mean inter-pulse duration above a threshold duration may be considered low quality. The threshold duration may be a duration in one of the following ranges: 10-20 seconds, 20-30 seconds, 30-40 seconds, 40-50 seconds, 50-60 seconds, 60-70 seconds, 70-80 seconds, 80-90 seconds, or 90-100 seconds. For example, the threshold duration may be 42 seconds. As another example, recognition segments with a dye purity below a threshold dye purity may be considered low quality. The threshold dye purity may be a dye purity in one of the following ranges: 0-0.1, 0.1-0.2, 0.2-0.3, 0.3-0.4, 0.4-0.5, or another suitable range. For example, the threshold dye purity may be 0.15. As another example recognition segments with dye composition distance above a threshold dye composition distance may be considered low quality. The threshold dye composition distance may be a dye composition distance in one of the following ranges: 0-0.1, 0.1-0.2, 0.2-0.3, 0.3-0.4, 0.4-0.5, 0.5-0.6, 0.6-0.7, or another suitable range. For example, the threshold dye composition distance may be 0.47. This filtering may ensure that reads with misidentified, low-quality recognition segments can still align. If a valid recognition segment is accidentally filtered out by this mechanism, the read may still align, albeit with a deletion penalty for a reference state that would have aligned to the removed recognition segment.

In some embodiments, the alignment module 108 may be configured to calculate a gap score s_{gap} as a contribution to an alignment score. The gap score may reward alignments in which regions of no pulsing between recognition segments line up with states of a reference peptide sequence that are expected to be invisible due to a lack of a specific recognizer for that state. In some embodiments, the gap score may depend on a context of the alignment and may be bounded by realistic time frames that correspond to unrecognized amino acids. In some embodiments, the alignment module 108 may be configured to determine the gap score based on the following conditions.
If two adjacent recognition segments align to a same reference state and there is a gap of at least a threshold amount of time between them, then s_{gap}=−w_{gap}×0.5, where w_{gap} is a gap score weight. The threshold amount of time may be an amount of time in one of the following ranges: 600-700 seconds, 700-800 seconds, 800-900 seconds, 900-1000 seconds, 1000-1100 seconds, 1100-1200 seconds, 1200-1300 seconds, 1300-1400 seconds, 1400-1500 seconds, 1500-1600 seconds, 1600-1700 seconds, 1700-1800 seconds, 1800-1900 seconds, 1900-2000 seconds, or another suitable time range. For example, the threshold amount of time may be 1200 seconds.
If two recognition segments are aligned to adjacent states and there is a gap of less than a threshold amount of time, then s_{gap}=−w_{gap}×0.5. The threshold amount of time may be an amount of time in one of the following ranges: 100-200 seconds, 200-300 seconds, 300-400 seconds, 400-500 seconds, 500-600 seconds, 600-700 seconds, 700-800 seconds, 800-900 seconds, 900-1000 seconds, or another suitable time range. For example, the threshold amount of time may be 300 seconds.
If there is exactly one skipped residue in the reference (whether an invisible residue or a deleted visible residue) between two adjacent recognition segments and there is a gap that falls within a particular range of time durations (e.g., between 60 and 3000 seconds), then s_{gap}=w_{gap}×0.5.
If there are two or more skipped residues in the reference between adjacent recognition segments and there is a gap of greater than a threshold amount of time, then s_{gap}=w_{gap}×0.5. The threshold amount of time may be an amount of time in one of the following ranges: 600-700 seconds, 700-800 seconds, 800-900 seconds, 900-1000 seconds, 1000-1100 seconds, 1100-1200 seconds, 1200-1300 seconds, 1300-1400 seconds, 1400-1500 seconds, 1500-1600 seconds, 1600-1700 seconds, 1700-1800 seconds, 1800-1900 seconds, 1900-2000 seconds, or another suitable time range. For example, the threshold amount of time may be 1200 seconds.
In all other cases, s_{gap}=0.

This formulation may have the effect of penalizing alignments that imply a single state has been oversplit when the recognition segments are not adjacent in the read and rewarding alignments where a gap between recognition segments is consistent with skipped states in the reference, as these skipped states may create periods of no pulsing in the read.

One potential issue with the scoring algorithm is that if a state is oversplit, this may increase a total number of recognition segments in the read, thereby increasing a maximum possible alignment score. Since downstream applications may filter alignments to those above some minimum alignment score threshold, this scoring artifact may have the effect of increasing a prevalence of oversplit recognition segments, which may introduce bias. To ameliorate this issue, in some embodiments, the alignment module 103 may be configured to give each recognition segment a score weight w_{RS} equal to 1/n, where n is a number of adjacent recognition segments in the read that have been labeled as a same recognizer. For example, if a recognition segment recognizer sequence is ABBCB, then the state weights are 1, ½, ½, 1, 1.

In some embodiments, the alignment module 108 may be configured to calculate a final alignment score S_{alignment} as a sum of each state's match score and gap score multiplied by a recognition segment weight, plus a deletion penalty of any skipped state from the reference. For purposes of weighting, each contribution to the gap score may be associated with a subsequent recognition segment in the read. The final alignment score may be calculated as:

$${S}_{alignmen t} = {\sum }_{i}^{{N}_{R S s}} {w}_{R S}^{( i )} \times ( {s}_{match}^{( i )} + {s}_{g a p}^{( i )} ) + {\sum }_{j}^{{N}_{d e l e t i o n s}} {s}_{deletion}^{( j )}$$

In some embodiments, for a read to be a candidate for alignment, the read may be required to meet certain criteria that qualify it as a high quality read. Accordingly, the alignment module 108 may be configured to filter reads to obtain those that are of sufficient quality. In some embodiments, for a read to be of sufficient quality, at least a threshold number of recognizers may be required to be present among those assigned to recognition segments of the read. The threshold number of recognizers may be (e.g., 3, 4, 5, 6, 7, or 8 recognizers. For example, the threshold number of recognizers may be 3 recognizers. In some embodiments, a length of the read may be required to be at least a threshold length after collapsing adjacent recognition segments that share a recognizer (equivalently, a sum of recognition segment weights may be required to be greater than or equal to the threshold length). The threshold length may be 2, 3, 4, 5, 6, 7, 8, 10, or another suitable threshold length. For example, the threshold length may be 4. These filters may ensure that only reads containing enough information to reliably distinguish between peptide references are passed to the alignment module 108.

FIG. 4B illustrates example matching of recognizers assigned to recognition segments of a read to amino acid residues of a reference peptide sequence, according to some embodiments of the technology described herein. Referring to FIG. 4B, the reference peptide sequence 404A is shown as a horizontal sequence of amino acid residues labeled R, F, N, E, L, N, F, D, I, S, R, and continues with additional residues. The read 400 comprises a first recognition segment 400A, a second recognition segment 400B, a third recognition segment 400C, and a fourth recognition segment 400D. Each of the recognition segments 400A, 400B, 400C, 400D may indicate a particular time period in which fluorescently tagged NAA recognizer(s) were binding to a particular NAA of a peptide being sequenced.

As illustrated in FIG. 4B, a plurality of recognizers are shown on the left side of the figure, including recognizer(s) 412A, recognizer(s) 412B, recognizer(s) 412C, and recognizer(s) 412D. Additional recognizers may be present as indicated by a vertical ellipsis in FIG. 4B. Arrows extend from the recognizers 412A, 412B, 412C, 412D to corresponding amino acid residues in the reference peptide sequence 404A, indicating binding relationships between the recognizers and specific amino acids. The recognizer(s) 412A may correspond to the R amino acid residue in the reference peptide sequence 404A. The recognizer(s) 412B may correspond to the F amino acid residues in the reference peptide sequence 404A. The recognizer(s) 412C may correspond to the N amino acid residues in the reference peptide sequence 404A. The recognizer(s) 412D may correspond to the D amino acid residue in the reference peptide sequence 404A.

With continued reference to FIG. 4B, the first recognition segment 400A corresponds to the recognizer(s) 412A, the second recognition segment 400B corresponds to the recognizer(s) 412B, the third recognition segment 400C corresponds to the recognizer(s) 412C, and the fourth recognition segment 400D corresponds to the recognizer(s) 412D. The alignment module 108 may be configured to match fluorescently tagged NAA recognizers assigned to the recognition segments 400A, 400B, 400C, 400D to expected fluorescently tagged NAA recognizers for amino acid residues in the reference peptide sequence 404A. For example, the alignment module 108 may be configured to match the recognizer(s) 412A assigned to the first recognition segment 400A to an expected recognizer for the R amino acid residue in the reference peptide sequence 404A.

In some embodiments, the alignment module 108 may be configured to determine alignment scores for candidate alignments by obtaining expected light pulse durations for certain amino acid motifs in the reference peptide sequence 404A. The alignment module 108 may be configured to obtain expected light pulse durations for amino acid motifs based on amino acid residues in the reference peptide sequence 404A that are matched with recognition segments. For example, the alignment module 108 may identify, for each of the matched amino acid residues, an amino acid motif composed of the amino acid residue and a particular number (e.g., 4 or 5) of preceding amino acid residues in the reference peptide sequence 404A. The alignment module 108 may be configured to access the reference alignment data 114 to obtain the expected light pulse durations for the amino acid motifs. For example, the alignment module 108 may look up amino acid motifs identified in the reference peptide sequence 404A in the reference alignment data 114 to obtain expected light pulse durations for respective amino acid residues. For each recognition segment in the read 400, the alignment module 108 may be configured to compare light pulse durations of the recognition segment to expected light pulse durations obtained for respective amino acid residues (e.g., from the reference alignment data 114) in the reference peptide sequence 404A with which the recognition segment is aligned.

In some embodiments, comparing the light pulse durations of the recognition segments to the expected light pulse durations of the respective amino acid residues in the reference peptide sequence 404A may comprise determining differences between mean light pulse durations of the recognition segments and the expected light pulse durations of the respective amino acid residues in the reference peptide sequence 404A. For example, the alignment module 108 may be configured to calculate a difference between a mean light pulse duration of the first recognition segment 400A and an expected light pulse duration obtained for the R amino acid residue in the reference peptide sequence 404A. The alignment module 108 may be configured to determine a component of an alignment score (e.g., s_{match} described above) using the differences between the mean light pulse durations of the recognition segments and the expected light pulse durations of the respective amino acid residues in the reference peptide sequence 404A. The alignment module 108 may be configured to determine an alignment score for a candidate alignment using a result of comparing the light pulse durations of the recognition segments to the expected light pulse durations of the respective amino acid residues in the reference peptide sequence 404A.

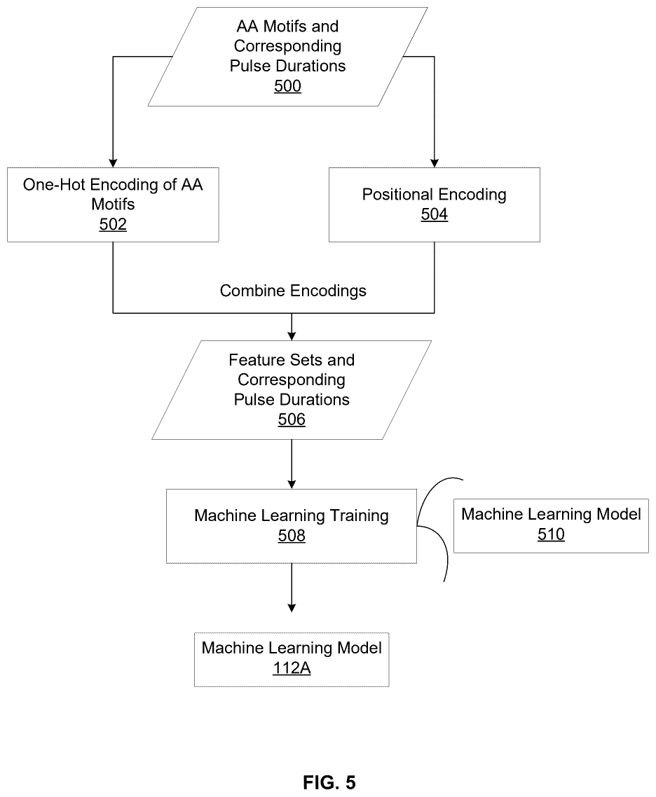

FIG. 5 illustrates training of the machine learning model 112A used to predict pulse durations for amino acid (AA) motifs in reference alignment data 114, according to some embodiments of the technology described herein. Referring to FIG. 5, the process begins with amino acid motifs and corresponding pulse durations 500, which serve as input data for the training process. In some embodiments, the amino acid motifs and corresponding pulse durations 500 may comprise sequences of amino acid residues (e.g., of 4, 5, 6, or 7 amino acid residues) paired with empirically measured pulse duration values obtained for those sequences from sequencing runs. In some embodiments, the use of sequences that are a particular length may be based on the hypothesis that pulse duration is influenced by an N-terminal residue and a certain number of downstream residues in a direction from the N-terminus towards the C-terminus.

As illustrated in FIG. 5, the amino acid motifs and corresponding pulse durations 500 are processed through two encoding paths. A first path involves a one-hot encoding of amino acid motifs 502, which converts amino acid identities into a binary vector representation where each amino acid is represented by a unique position in the vector. For example, in the one-hot encoding of amino acid residues 502, each of the 20 canonical amino acids may be represented by a vector having a value of 1 at a position corresponding to that amino acid and values of 0 at all other positions.

With continued reference to FIG. 5, a second path involves a positional encoding 504, which captures a position of each amino acid within a an amino acid motif using sinusoidal functions to generate deterministic embeddings tied to each position index. The positional encoding 504 may incorporate sequence position into feature representations without reliance on recurrent or convolutional operations. The positional encoding 504 may be adapted from a Transformer model architecture, using sine and cosine functions at different frequencies to produce a deterministic, continuous set of embeddings tied to each position index. For example, for an input sequence of length 5 and a feature dimension D_{model}, the positional encoding 504 may be defined elementwise as:

$$PE ( pos , 2 i ) = sin ( \frac{pos}{1000 {0}^{( \frac{2 i}{{D}_{model}} )}} ) , PE ( pos , 2 i + 1 ) = cos ( \frac{pos}{1000 {0}^{( 2 i / {D}_{model} )}} )$$

where pos is a position within the motif and i indexes a dimension. The positional encoding 504 may recapitulate a largely identity-independent effect that peptide sequence position has on pulse duration, which arises from a local environment surrounding each recognizer-bound peptide residue. As this per-position environment remains consistent from peptide to peptide, the positional encoding 504 may imbue the machine learning model 112A with a trainable parameter aimed at recapitulating a contribution of individual peptide positions on pulse duration that are inherent to a recognizer binding mode.

As shown in the example of FIG. 5, the one-hot encoding of amino acid motifs 502 and the positional encoding 504 are combined to generate feature sets and corresponding pulse durations 506. The feature sets and corresponding pulse durations 506 integrate amino acid identity information with positional context information, creating a representation that captures both what amino acids are present and where they are located within the motif. For each of the amino acid motifs, the reference alignment data generation module 112 may be configured to generate a set of features for the amino acid motif at least in part by combining the one-hot encoding and the sinusoidal positional encoding.

With continued reference to FIG. 5, the feature sets and corresponding pulse durations 506 are provided to a machine learning training 508 operation, which uses the combined feature representations along with corresponding pulse duration measurements to train a model. The machine learning training 508 may utilize a machine learning model 510 as a starting point or reference architecture. The machine learning training 508 produces the trained machine learning model 112A as output. The machine learning model 112A may be configured to predict pulse durations for amino acid motifs based on learned relationships between sequence features and pulse duration measurements.

The machine learning model 112A may be any suitable machine learning model. In some embodiments, the machine learning model 112A may comprise a neural network. The neural network may comprise a plurality of fully connected layers. A first layer of the plurality of fully connected layers may be configured to receive a combination of an input one-hot encoding with an input sinusoidal positional encoding generated for a particular amino acid motif. An output layer of the plurality of fully connected layers may be configured to output a pulse duration prediction for the particular amino acid sequence.

In some embodiments, the neural network may comprise fully connected layers with gradually reduced dimensionality. For example, the fully connected layers may have dimensions of 128→64→16, progressing from a higher dimensionality to a lower dimensionality to enhance computational efficiency and parameter regularization. In some embodiments, each of a first two layers of the fully connected layers may be normalized using batch normalization. In some embodiments, the fully connected layers may be regularized with a dropout rate. The dropout rate may be in one of the following ranges: 0.1-0.2, 0.2-0.3, 0.3-0.4, 0.4-0.5, 0.5-0.6, or another suitable range. For example, the dropout rate may be 0.4. In some embodiments, ReLU activation functions may be employed in all hidden layers of the neural network to improve training stability and accelerate convergence. A single-value regression estimate may be produced by the output layer of the neural network.

In some embodiments, training data for the machine learning model 112A may be curated from sequencing runs comprising proteins and synthetic peptides that are sequenced and aligned against respective reference sequences. Entries may be included in the training data when a minimum number of recognition events per run are observed. The minimum number of recognition events may be in one of the following ranges: 50-100, 100-150, 150-200, 200-250, or another suitable range. For example, the minimum number of recognition events may be 100 recognition events per run. In some embodiments, entries may be included in the training data when the entries constitute at least a threshold percentage of alignment coverage at a position. The threshold percentage may be in one of the following ranges: 5%-10%, 10%-15%, 15%-20%, 20%-25%, or another suitable range. For example, the threshold percentage may be 10% of alignment coverage at a position.

In some embodiments, amino acid motifs identified in aligned recognitions may be clustered by Levenshtein distance. Resulting clusters of unique amino acid motifs and corresponding pulse duration measurements may be distributed across training, validation, and test sets to ensure balanced and representative data partitioning. This clustering approach may prevent similar motifs from appearing in both training and test sets, which may improve generalization of the trained machine learning model 112A. In some embodiments, the trained machine learning model 112A may be applied to predict pulse durations for all possible amino acid motifs starting with any of 13 N-terminally recognized amino acids and containing any of 20 canonical amino acids in each of a number of remaining positions (e.g., 4 positions or 5 positions). For amino acid motifs of 5 amino acid residues, this approach may produce predictions for 2,080,000 (13×20⁴) unique motifs. In cases where a pulse duration has been previously measured for a corresponding amino acid motif, an empirical value may be used. In cases where a pulse duration has not been previously measured, a predicted value from the machine learning model 112A may be used. Combining these results, the reference alignment data 114 may be generated as a comprehensive kinetic database pairing each amino acid motif with a corresponding pulse duration for use in scoring alignments.

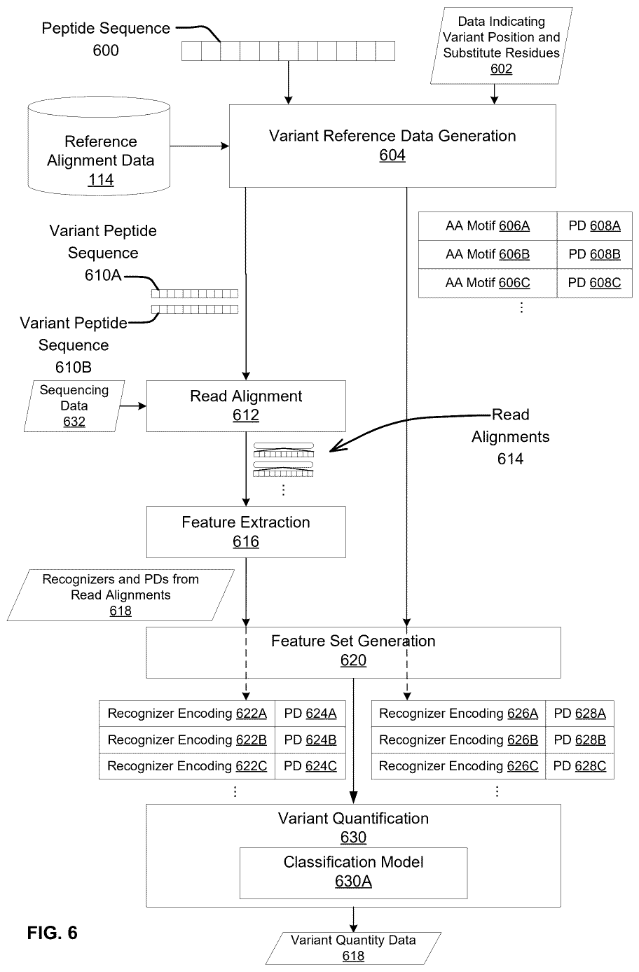

FIG. 6 illustrates a system diagram for variant detection and quantification using sequencing data, according to some embodiments of the technology described herein. Referring to FIG. 6, the AA variant detection module 110 may be configured to receive a peptide sequence 600 and variant position and substitute residues data 602 as inputs to a variant reference data generation 604 process. The peptide sequence 600 may comprise an amino acid residue sequence of a reference peptide for which variants are to be detected. The variant position and substitute residues data 602 may indicate a position within the peptide sequence 600 at which a variant occurs and one or more substitute amino acid residues that may be present at the variant position. The variant reference data generation 604 also receives the reference alignment data 114.

As illustrated in FIG. 6, the variant reference data generation 604 may be configured to identify amino acid motifs 606A, 606B, 606C and corresponding pulse durations 608A, 608B, 608C using the reference alignment data 114. The variant reference data generation 604 may be configured to retrieve expected variant amino acid residues and corresponding upstream amino acid residues, along with corresponding pulse durations from the reference alignment data 114. In some embodiments, the variant reference data generation 604 may be configured to generate subsequences spanning from a variant site up to a particular number of residues upstream. The particular number of residues may be 1, 2, 3, 4, 5, 6, 7, or 8 residues. For example, the particular number of residues may be 4 residues upstream. The variant reference data generation 604 may look up the subsequences in the reference alignment data 114 to identify matching amino acid motifs. The variant reference data generation 604 may read out the matched amino acid motifs. As shown in FIG. 6, the variant reference data generation 604 obtains amino acid motif 606A with a pulse duration 608A, amino acid motif 606B with pulse duration 608B, and amino acid motif 606C with pulse duration 608C from the reference alignment data 114. Each of the amino acid motifs 606A, 606B, 606C may represent an amino acid motif at a particular position corresponding to a variant site, and each of the pulse durations 608A, 608B, 608C may represent an expected pulse duration for the amino acid motif.

With continued reference to FIG. 6, the amino acid variant detection module 110 may be configured to generate a variant peptide sequence 610A and a variant peptide sequence 610B based on the variant information provided in the variant position and substitute residues data 602. The variant peptide sequence 610A may represent a first variant of the peptide having a first amino acid residue at the variant position, and the variant peptide sequence 610B may represent a second variant of the peptide having a second amino acid residue at the variant position. In some embodiments, the AA variant detection module 110 may be configured to categorize variants into three types based on visibility of amino acids at the variant site. A first type may include visible-to-visible variants where both amino acid residues at the variant position have corresponding recognizers. A second type may include visible-to-invisible or invisible-to-visible variants where one amino acid residue at the variant position has a corresponding recognizer and another amino acid residue does not. A third type may include invisible-to-invisible variants where neither amino acid residue at the variant position has a corresponding recognizer.

As illustrated in FIG. 6, sequencing data 632 is provided to a read alignment 612 process along with the variant peptide sequences 610A and 610B. The sequencing data 632 may comprise sequencing data obtained from sequencing a sample containing one or more variants of the peptide. The read alignment 612 may be configured to align reads from the sequencing data 632 to each of the variant peptide sequences 610A and 610B. In some embodiments, the read alignment 612 may be configured to use alignments scoring at least a threshold score in subsequent detection processing to reduce the influence of ambiguous reads. The threshold score may be in one of the following ranges: 2.0-2.5, 2.5-3.0, 3.0-3.5, 3.5-4.0, 4.0-4.5, 4.5-5.0, or another suitable range. For example, the threshold score may be 3.75. The read alignment 612 produces read alignments 614 as output.

With continued reference to FIG. 6, the read alignments 614 are processed by a feature extraction 616 operation to obtain pulse durations extracted from read alignments 618. The feature extraction 616 may be configured to extract kinetic features such as pulse duration from the read alignments 614. The pulse durations extracted from read alignments 618 may comprise pulse duration measurements for recognition segments aligned to positions of variants in the variant peptide sequences 610A, 610B.

As shown in FIG. 6, the pulse durations extracted from read alignments 618 are provided to a feature set generation 620 operation. The feature set generation 620 may be configured to construct a multidimensional feature space by integrating aggregated positional kinetics with one-hot encoded binary recognizer features. In some embodiments, the feature set generation 620 may be configured to integrate positional kinetics spanning from the variant site up to four residues upstream with one-hot encoded binary recognizer features. These inputs may capture recognizer read variation and a context influencing pulse duration.

As illustrated in FIG. 6, the feature set generation 620 generates feature sets using recognizers and pulse durations obtained from the read alignments 618, and feature sets using amino acid motifs and corresponding expected pulse durations obtained from the reference alignment data 114. Each feature set may include a recognizer encoding paired with a pulse duration. A first group of feature sets, generated using the read alignments 618, includes a recognizer encoding 622A with an extracted pulse duration 624A, a recognizer encoding 622B with an extracted pulse duration 624B, and a recognizer encoding 622C with an extracted pulse duration 624C. A second group of feature sets, generated using the amino acid motifs and corresponding expected pulse durations, includes a recognizer encoding 626A with an extracted pulse duration 628A, a recognizer encoding 626B with an extracted pulse duration 628B, and a recognizer encoding 626C with an extracted pulse duration 628C. The recognizer encodings 622A, 622B, 622C may comprise one-hot encoded representations of recognizers assigned to recognition segments at respective positions. The extracted pulse durations 624A, 624B, 624C may comprise pulse duration measurements extracted from the read alignments 614 for recognition segments at respective positions. The recognizer encodings 626A, 626B, 626C may comprise one-hot encoded representations of recognizers associated with amino acid motifs (e.g., expected for particular amino acid residues in the amino acid motifs). The pulse durations 628A, 628B, 628C may comprise expected pulse durations for the amino acid motifs obtained from the reference alignment data 114.

With continued reference to FIG. 6, the feature sets are provided to a variant quantification 630 operation, which employs a classification model 630A to process the feature sets and produce variant quantity data as output. In some embodiments, the classification model 630A may comprise a two-component Gaussian Mixture Model (GMM) for clustering reads into variant populations. The two-component GMM may be trained using the feature sets, with initial centroids guided by expected kinetic profiles from the reference alignment data 114 and recognizer identities (e.g., the second group of feature sets generated from the amino acid motifs and corresponding expected pulse durations). In some embodiments, the variant quantification 630 may be configured to select a single position showing a largest pulse duration difference between variant peptides as a primary feature for clustering. For example, for a visible-to-visible variant, the variant quantification 630 may select the variant position itself as the primary feature when the variant position shows the largest difference in pulse duration between the two variants. For other variant types, the variant quantification 630 may select an upstream position that exhibits a pronounced difference in pulse duration as the primary feature. In some embodiments, applying the trained GMM to the feature sets may yield predicted population identities for all data points. The variant quantification 630 may be configured to calculate a ratio of variant populations from the GMM populations. The AA variant data 116 output by the AA variant detection module 110 may include the calculated ratio of variant populations, providing quantification of the relative abundance of each variant in the sample.

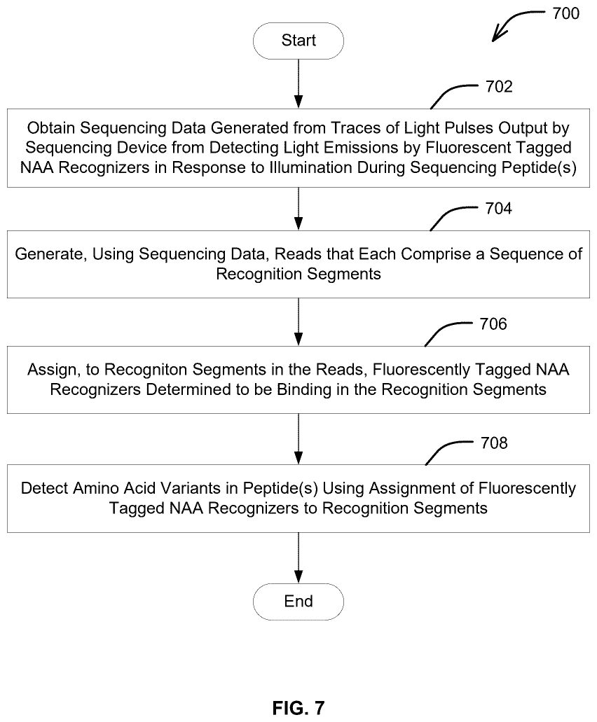

FIG. 7 illustrates a flowchart for a process 700 for detecting amino acid variants in peptides using sequencing data, according to some embodiments of the technology described herein. In some embodiments, the process 700 may be performed by the sequencing data processing system 100 described herein with reference to FIGS. 1A-6. Referring to FIG. 7, the process 700 begins at a Start node and proceeds through a series of blocks that implement the variant detection functionality described herein with reference to the sequencing data processing system 100.

As illustrated in FIG. 7, the process 700 proceeds from the Start node to a block 702. At the block 702, the system obtains sequencing data that was generated from traces of light pulses output by a sequencing device (e.g., the sequencing device 120) from detecting light emissions by fluorescently tagged NAA recognizers in response to illumination during sequencing of peptide(s). The sequencing data obtained at the block 702 may comprise light pulse durations of the light pulses and inter-pulse durations between successive ones of the light pulses. In some embodiments, the system may obtain the sequencing data from the sequencing device 120 performing NGPS of a peptide sample (e.g., the peptide sample 122). The sequencing data may be generated from binding events between fluorescently tagged NAA recognizers and peptides immobilized on a semiconductor chip of the sequencing device 120.

With continued reference to FIG. 7, the process 700 proceeds to a block 704. At the block 704, the system generates reads using the sequencing data, with each read comprising a sequence of recognition segments. Each recognition segment may indicate a particular time period in which one or more of the fluorescently tagged NAA recognizers were binding to a particular NAA of the peptide(s) being sequenced. In some embodiments, generating the reads at the block 704 may comprise segmenting light pulse traces into proto-recognition segments based on inter-pulse durations, and subdividing the proto-recognition segments into recognition segments based on comparisons of light pulse properties between sequential pairs of light pulse windows (e.g., as described herein with reference to FIGS. 2A-2C). The block 704 may be performed by the pulse identification module 102 and the pulse segmentation module 104 of the sequencing data processing system 100.

As further shown in FIG. 7, the process 700 proceeds to block 706. At the block 706, the system assigns fluorescently tagged NAA recognizers determined to be binding in the recognition segments to the recognition segments in the reads. In some embodiments, assigning the fluorescently tagged NAA recognizers at the block 706 may comprise obtaining fluorescence data for each recognition segment and identifying, using the fluorescence data, fluorescently tagged NAA recognizer(s) from among a set of candidate fluorescently tagged NAA recognizers for assignment to the recognition segment (e.g., as described herein with reference to FIGS. 3A-3B). The fluorescence data may indicate a detected fluorescence intensity and fluorescence decay of a fluorescent dye of the fluorescently tagged NAA recognizer(s) binding in the recognition segment. In some embodiments, the block 706 may be performed using the classification model 106A of the recognizer assignment module 106 (e.g., as described herein with reference to FIGS. 3A-3B).

With continued reference to FIG. 7, the process 700 proceeds to a block 708. At the block 708, the system detects amino acid variants in peptide(s). In some embodiments, the block 710 may be performed by the AA variant detection module 110 of the sequencing data processing system 100.

In some embodiments, detecting the amino acid variants at the block 710 may comprise generating reference profiles for variant peptide sequences, aligning reads to the variant peptide sequences (e.g., using the aligment module 108), extracting kinetic features (e.g., light pulse properties) from the alignments, generating feature sets combining recognizer encodings with extracted pulse durations, and applying a classification model (e.g., a two-component GMM) to quantify variant populations (e.g., as described herein with reference to FIG. 5).

In some embodiments, the system aligns the read(s) to reference peptide sequence(s) to obtain peptide alignment(s). In some embodiments, aligning the reads at the block 708 may comprise using the fluorescently tagged NAA recognizers assigned to recognition segments of the reads, light pulse durations in the recognition segments, and inter-pulse durations to score candidate alignments and select alignments (e.g., as described herein with reference to FIGS. 4A-4B). The block 710 may comprise generating multiple candidate alignments with reference peptide sequences, determining alignment scores for the candidate alignments, and selecting one of the candidate alignments as an alignment based on the alignment scores. In some embodiments, the alignment scores may be determined using match scores based on comparisons between observed pulse durations and expected pulse durations obtained from the reference alignment data 114, deletion scores for visible states not aligned to recognition segments, and gap scores based on spacing between recognition segments relative to reference peptide sequences. The aligning may be performed by the alignment module 108 of the sequencing data processing system 100.

In some embodiments, the block 708 may involve performing amino acid variant detection without using alignment(s) from the alignment module 108. In such embodiments, the alignment module 108 may not be used to generate alignments (e.g., alignment 136) or the AA variant detection module 110 may not obtain alignments from the alignment module 108. In some embodiments, the the amino acid variant detection may involve performing detection using reads and an assignment of fluorescently tagged NAA recognizers to recognition segments of reads including the read 132. In some embodiments, block 708 may involve constructing a multidimensional feature space by integrating aggregated positional kinetics of variant sites. These inputs may capture recognizer read variation. Block 708 may involve using the assignment of fluorescently tagged NAA recognizers to recognition segments to determine amino acid variant identities of the plurality of reads without requiring alignment to reference peptide sequences.

In some embodiments, block 708 may involve determining amino acid variant identities of reads using a trained machine learning model. The trained machine learning model may process features derived from the recognizer assignments and, optionally, light pulse properties (e.g., pulse durations and/or inter-pulse durations) of the recognition segments to classify each read as corresponding to a particular amino acid variant. In some embodiments, the trained machine learning model may comprise a classification model, and the AA variant detection module 110 may be configured to train the classification model by clustering the plurality of reads to obtain multiple classes each corresponding to a particular amino acid variant. For example, a multi-component GMM (e.g., two-component GMM) may be trained using these features, with initial centroids guided by the expected kinetic profiles from the kinetic database and recognizer identities. Applying the trained GMM to the dataset yields amino acid variant identities for the reads. The block 708 may involve classifying each read into one of the classes to obtain an amino acid variant identity of the read. In some embodiments, the trained machine learning model may comprise a pre-trained machine learning model. For example, the machine learning model may have been trained using reads labeled with known amino acid variants to which the reads correspond. A supervised learning algorithm may be applied to the labeled reads to obtain the trained machine learning model.

Example machine learning models that may be used to classify reads with amino acid variant identities include a Gaussian Mixture Model (GMM) classifier, a K-means clustering model, a support vector machine (SVM) classifier, a K-nearest neighbors (KNN) classifier, a random forest classifier, a logistic regression classifier, a neural network classifier, a Naive Bayes classifier, a decision tree classifier, a gradient boosting classifier, a linear discriminant analysis (LDA) classifier, a quadratic discriminant analysis (QDA) classifier, a hidden Markov model (HMM), a recurrent neural network (RNN), a long short-term memory (LSTM) network, a convolutional neural network (CNN), or another suitable classifier. In some embodiments, the block 708 may involve using dynamic time warping in combination with clustering algorithms to group reads based on temporal patterns in recognition segment sequences. The selection of a particular machine learning model may depend on factors such as the complexity of the variant discrimination task, the dimensionality of the feature space, the amount of training data available, and computational efficiency requirements.

At block 708, in some embodiments, the system may produce the AA variant data 116 as output, which may include information about detected amino acid variants such as positions and quantification of the variants. In some embodiments, the AA variant data 116 may include a ratio of a quantity of a particular peptide variant to a quantity of one or more other peptide variants.

In some embodiments, at block 708, detecting amino acid variant(s) may comprise identifying variant positions in a reference peptide sequence. For example, the system may identify positions in a read alignment where recognizers of recognition segments differ from expected recognizers of amino acid residues at the positions. The system may be configured to output indications of positions in an alignment where variants were identified. In some embodiments, detecting the amino acid variant(s) may comprise identifying substitute amino acid residues at variant positions. The system may be configured to identify, at variant positions, one or more amino acid residues associated with recognizers assigned to recognizer segments, and output the amino acid residues identified at the variant positions as substitute amino acid residue(s) at the variant positions.

With continued reference to FIG. 7, the process 700 concludes at an End node following completion of the block 708. The process 700 illustrates a sequential approach for processing sequencing data through multiple stages including data acquisition at the block 702, read generation at the block 704, recognizer assignment at the block 706, and variant detection at the block 708 to identify amino acid variants in peptides.

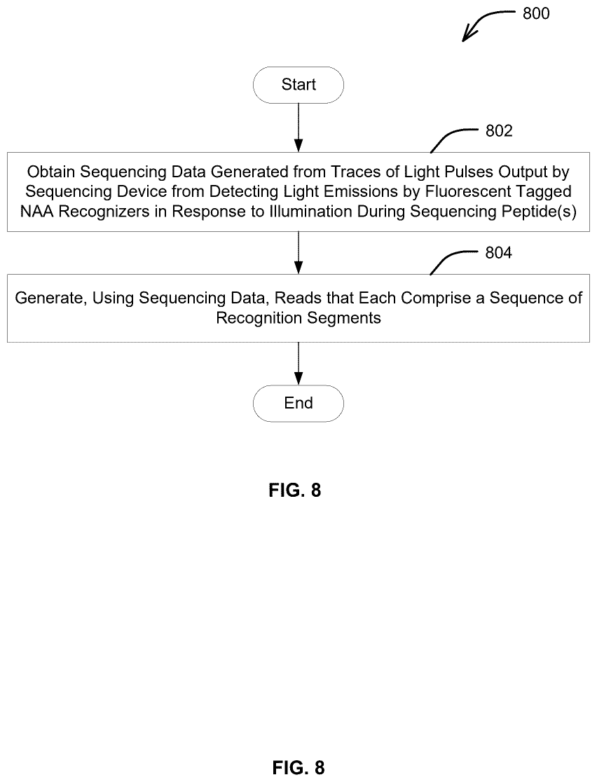

FIG. 8 illustrates a flowchart for a process 800 for generating reads using data obtained by a sequencing device, according to some embodiments of the technology described herein. In some embodiments, the process 800 may be performed by the sequencing data processing system 100 described herein with reference to FIGS. 1A-6. The process 800 may be performed by the pulse identification module 102 and the pulse segmentation module 104 of the sequencing data processing system 100. Referring to FIG. 8, the process 800 begins at a Start node and proceeds through a series of blocks that implement read generation functionality.

As illustrated in FIG. 8, the process 800 proceeds from the Start node to a block 802. At the block 802, the system obtains sequencing data as described with reference to block 702 of process 700 described herein with reference to FIG. 7. The sequencing data may be generated from traces of light pulses output by the sequencing device 120 from detecting light emissions by fluorescently tagged NAA recognizers in response to illumination during sequencing of peptide(s). In some embodiments, the sequencing data obtained at the block 802 may comprise light pulse durations of the light pulses and inter-pulse durations between successive ones of the light pulses. The sequencing data may be generated from binding events between fluorescently tagged NAA recognizers and peptides immobilized on a semiconductor chip of the sequencing device 120. In some embodiments, the pulse identification module 102 may perform the block 802 by receiving raw signal data from the sequencing device 120 and processing the raw signal data to identify light pulses and determine light pulse properties including pulse durations and inter-pulse durations.

With continued reference to FIG. 8, the process 800 proceeds to a block 804. At the block 804, the system generates reads using the sequencing data as described at block 704 of process 700 described herein with reference to FIG. 7. Each read may comprise a sequence of recognition segments. Each recognition segment may indicate a particular time period in which one or more of the fluorescently tagged NAA recognizers were binding to a particular NAA of the peptide(s) being sequenced. In some embodiments, generating the reads at the block 804 may comprise segmenting light pulse traces into proto-recognition segments based on inter-pulse durations, and subdividing the proto-recognition segments into recognition segments based on comparisons of light pulse properties between sequential pairs of light pulse windows. In some embodiments, the pulse segmentation module 104 may perform the block 804 by identifying boundaries where regions of active pulsing terminate and by detecting where pulsing properties change within proto-recognition segments to identify individual recognition segments.

Following completion of the block 804, the process 800 proceeds to an End node. The process 800 illustrates a structured approach for processing sequencing data to generate reads that can be used for subsequent analysis of peptide sequences, including recognizer assignment, alignment, and variant detection.

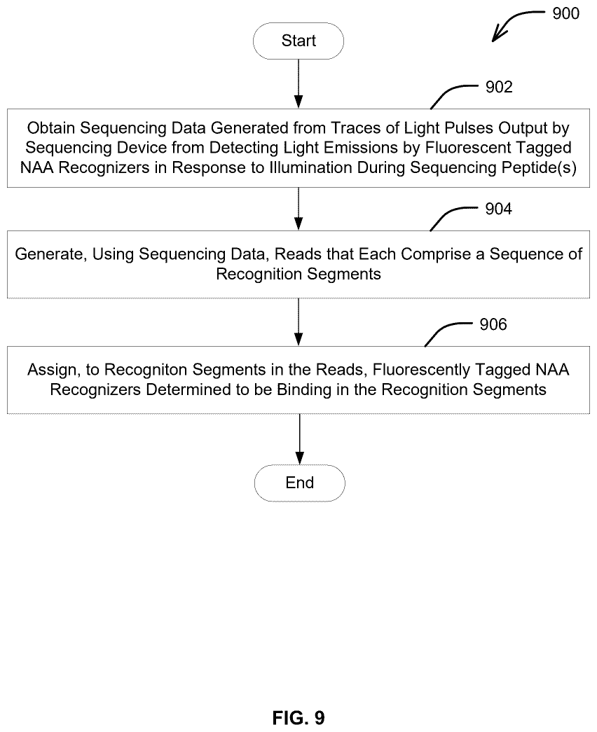

FIG. 9 illustrates a flowchart for a process 900 for identifying amino acid residue sequences in peptides using data obtained by a sequencing device, according to some embodiments of the technology described herein. In some embodiments, the process 900 may be performed by the sequencing data processing system 100 described herein with reference to FIGS. 1A-6. Referring to FIG. 9, the process 900 begins at a Start node and proceeds through a series of blocks that implement amino acid residue sequence identification functionality.

As illustrated in FIG. 9, the process 900 proceeds from the Start node to a block 902. At the block 902, the system obtains sequencing data that is generated from traces of light pulses output by the sequencing device 120 from detecting light emissions by fluorescently tagged NAA recognizers in response to illumination during sequencing of peptide(s). The sequencing data obtained at the block 902 may comprise light pulse durations of the light pulses and inter-pulse durations between successive ones of the light pulses. In some embodiments, the sequencing data may be generated from binding events between fluorescently tagged NAA recognizers and peptides immobilized on a semiconductor chip of the sequencing device 120. In some embodiments, the pulse identification module 102 may perform the block 902 by receiving raw signal data from the sequencing device 120 and processing the raw signal data to identify light pulses and determine light pulse properties including pulse durations and inter-pulse durations.

With continued reference to FIG. 9, the process 900 proceeds to a block 904. At the block 904, the system generates reads using the sequencing data, with each read comprising a sequence of recognition segments. Each recognition segment may indicate a particular time period in which one or more of the fluorescently tagged NAA recognizers were binding to a particular NAA of the peptide(s) being sequenced. In some embodiments, generating the reads at the block 904 may comprise segmenting light pulse traces into proto-recognition segments based on inter-pulse durations, and subdividing the proto-recognition segments into recognition segments based on comparisons of light pulse properties between sequential pairs of light pulse windows. In some embodiments, the pulse segmentation module 104 may perform the block 904 by identifying boundaries where regions of active pulsing terminate and by detecting where pulsing properties change within proto-recognition segments to identify individual recognition segments.

As further shown in FIG. 9, the process 900 proceeds to a block 906. At the block 906, the system assigns fluorescently tagged NAA recognizers determined to be binding in the recognition segments to the recognition segments in the reads. In some embodiments, assigning the fluorescently tagged NAA recognizers at the block 906 may comprise, for each of the recognition segments, obtaining fluorescence data for the recognition segment and identifying, using the fluorescence data, fluorescently tagged NAA recognizer(s) from among a set of candidate fluorescently tagged NAA recognizers for assignment to the recognition segment. The fluorescence data may indicate a detected fluorescence intensity and fluorescence decay of a fluorescent dye of the fluorescently tagged NAA recognizer(s) binding in the recognition segment. In some embodiments, the block 906 may be performed using the classification model 106A of the recognizer assignment module 106. The classification model 106A may process the fluorescence data to determine a classification of the fluorescence data as corresponding to the fluorescently tagged NAA recognizer(s).

With continued reference to FIG. 9, the process 900 proceeds to a block 908. At the block 908, the system identifies amino acid residue sequences in the peptide(s) using the fluorescently tagged NAA recognizers assigned to the recognition segments in the reads. In some embodiments, identifying the amino acid residue sequences at the block 908 may comprise determining, for each recognition segment, one or more amino acid residues associated with the fluorescently tagged NAA recognizer assigned to the recognition segment. Each fluorescently tagged NAA recognizer may be associated with one or more target amino acid residues that the recognizer binds. For example, a recognizer may bind to a single amino acid residue type, or a recognizer may bind to multiple amino acid residue types (e.g., a recognizer that binds to leucine, isoleucine, and valine). In some embodiments, the system may use the sequence of recognizers assigned to recognition segments in a read to infer a sequence of amino acid residues in the peptide. The system may output the identified amino acid residue sequences as part of the analysis of the peptide sample 122.

Following completion of the block 908, the process 900 concludes at an End node. The process 900 illustrates a sequence of steps for processing sequencing data to identify amino acid residue sequences, progressing from data acquisition at the block 902 through read generation at the block 904, recognizer assignment at the block 906, and amino acid sequence identification at the block 908.

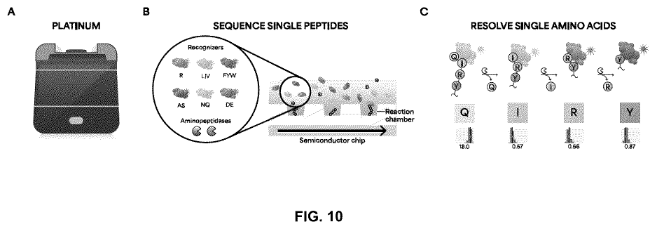

FIG. 10 illustrates a schematic overview of a next-generation protein sequencing system and workflow, according to some embodiments of the technology described herein. Referring to FIG. 10, the figure is organized into three panels labeled A, B, and C that depict different aspects of the sequencing system and process.

Panel A of FIG. 10 shows a sequencing instrument configured to perform single-molecule peptide sequencing. The sequencing instrument is depicted as a benchtop device with a body portion and a lid portion. The sequencing instrument may be configured to sequence single peptide molecules with single amino acid resolution. In some embodiments, the sequencing instrument may operate with a frame rate of approximately 60 ms for sampling signal data. In some embodiments, the sequencing instrument may operate with a run time of approximately 10 hours for peptide sequencing. The sequencing instrument may include a semiconductor chip on which peptides are immobilized for sequencing, an illumination source configured to excite fluorescent dyes conjugated to recognizers, and one or more detectors configured to detect light emissions from the fluorescent dyes in response to illumination.

With continued reference to FIG. 10, Panel B illustrates sequencing components used in the sequencing process. Panel B shows an expanded view of the sequencing components including multiple fluorescently tagged NAA recognizers. The recognizers shown in Panel B include a recognizer for R (arginine), a recognizer for LIV (leucine, isoleucine, and valine), a recognizer for FYW (phenylalanine, tyrosine, and tryptophan), a recognizer for AS (alanine and serine), a recognizer for NQ (asparagine and glutamine), and a recognizer for DE (aspartic acid and glutamic acid). The recognizers are depicted as colored shapes that interact with peptides in a reaction chamber. In some embodiments, a sequencing kit may include six NAA recognizers that recognize 13 of the 20 canonical amino acids. Each recognizer may be conjugated with a distinct fluorescent dye having a characteristic intensity and fluorescence decay lifetime. Panel B also shows aminopeptidases that sequentially cleave N-terminal amino acids from immobilized peptides, exposing successive amino acids for recognition by the recognizers. The reaction chamber is positioned above a semiconductor chip that detects light emissions from the fluorescently tagged recognizers during binding events.

As further shown in FIG. 10, Panel C depicts resolution of single amino acids during the sequencing process. Panel C shows a series of peptide molecules with attached recognizers represented as colored circles. Below each peptide representation are boxes containing single letter amino acid codes Q, I, R, and Y, with corresponding signal intensity bars showing values of 18.0, 0.57, 0.56, and 0.87 respectively. The workflow depicted in Panel C demonstrates how binding of dye-labeled NAA recognizers generates kinetic information indicating which amino acid is being detected at each position along the peptide sequence. The signal intensity values may correspond to pulse duration measurements or other kinetic properties that characterize the binding interaction between a particular recognizer and a particular amino acid residue. The variation in signal intensity values across different amino acid positions reflects differences in binding kinetics between different recognizer-amino acid combinations.

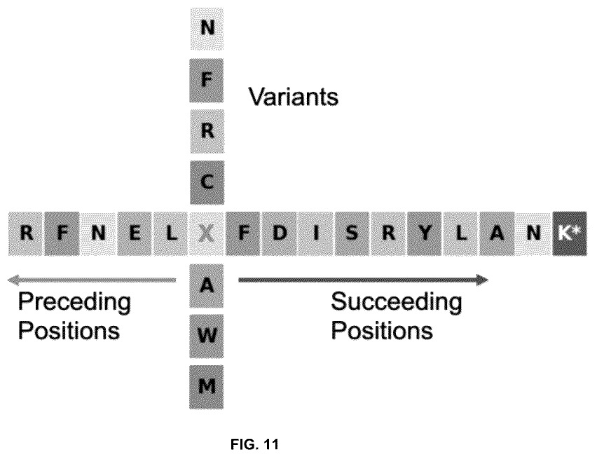

FIG. 11 illustrates a schematic diagram of a peptide sequence design with amino acid variants at a specific position, according to some embodiments of the technology described herein. Referring to FIG. 11, a horizontal row of squares represents a peptide sequence reading from left to right as R, F, N, E, L, X, F, D, I, S, R, Y, L, A, N, and K, where X denotes a variable position and K indicates a modified lysine residue. The modified lysine residue may comprise an azido-lysine modification that enables compatibility with library preparation for immobilization of the peptides on a semiconductor chip surface during sequencing.

As illustrated in FIG. 11, a vertical column of squares intersects the horizontal sequence at the X position, displaying seven possible amino acid variants that can occupy the sixth position in the peptide sequence. The amino acid variants N, F, R, and C extend upward from the X position, and the amino acid variants A, W, and M extend downward from the X position. The seven distinct amino acid variants at the sixth position include asparagine (N), phenylalanine (F), arginine (R), cysteine (C), alanine (A), tryptophan (W), and methionine (M). Each of the seven amino acid variants at the sixth position results in a distinct peptide sequence while maintaining identical flanking sequences on both sides of the variant position.

With continued reference to FIG. 11, an arrow labeled “Preceding Positions” points leftward from the X position, indicating amino acid residues located N-terminal to the variant site. The preceding positions include the amino acid residues R, F, N, E, and L at positions one through five of the peptide sequence. A second arrow labeled “Succeeding Positions” points rightward from the X position, indicating amino acid residues located C-terminal to the variant site. The succeeding positions include the amino acid residues F, D, I, S, R, Y, L, A, N, and K at positions seven through sixteen of the peptide sequence.

In some embodiments, the placement of the variant at the sixth position may be configured to capture kinetic variations associated with up to five preceding residues. The kinetic variations may arise from interactions between NAA recognizers and downstream positions relative to the N-terminal amino acid being recognized. In some embodiments, at least four amino acids N-terminal to the substitution site can be recognized by fluorescently tagged NAA recognizers, with three of these four amino acids being uniquely identifiable. For example, in the peptide sequence shown in FIG. 11, the amino acid residues R, F, N, E, and L at positions one through five preceding the variant site may include amino acid residues that are recognized by distinct fluorescently tagged NAA recognizers. The recognizable amino acid residues N-terminal to the variant site may facilitate variant calling in binary mixtures by providing kinetic signatures that differ between variant peptides based on the amino acid residue present at the sixth position.

In some embodiments, the peptide sequence design shown in FIG. 11 may be used to generate synthetic peptides for sequencing on an NGPS instrument. Each of the seven peptide variants may follow the sequence RFNELXFDISRYLANK, where X is substituted with one of F, W, R, M, N, A, or C. The C-terminal azido-lysine modification may enable immobilization of the peptides on a semiconductor chip surface through strain-promoted alkyne-azide cycloaddition click chemistry. In some embodiments, binary mixtures of peptide variants may be generated for analysis, such as asparagine to alanine (N6A), phenylalanine to tryptophan (F6W), arginine to methionine (R6M), and cysteine to methionine (C6M) variant pairs.

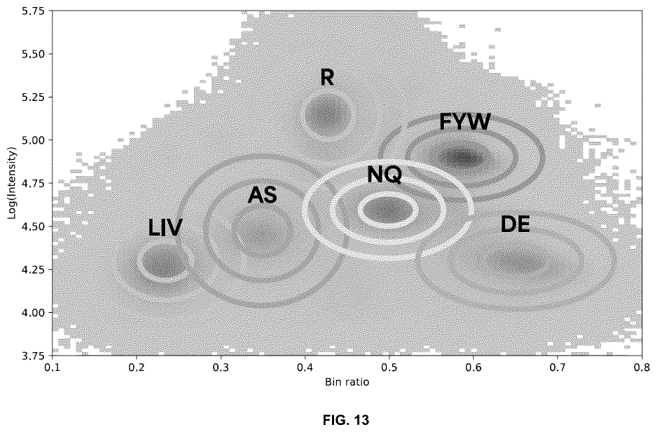

FIG. 13 illustrates a scatter plot showing a distribution of pulse bin ratios and log-intensities from a representative sequencing run, with a dye classification Gaussian Mixture Model overlaid, according to some embodiments of the technology described herein. Referring to FIG. 13, the scatter plot has a horizontal axis representing the bin ratio, ranging from approximately 0.1 to 0.8, and a vertical axis representing the log of intensity, ranging from approximately 3.75 to 5.75. The background of the scatter plot shows a density distribution of data points represented in varying shades, with darker regions indicating higher concentrations of pulses detected during the sequencing run.

As illustrated in FIG. 13, the bin ratio may serve as a proxy for fluorescence decay lifetime in downstream data analysis. The bin ratio may be calculated as a ratio of measurements from two collection windows with different delays following laser illumination. For example, a sequencing device may measure fluorescence decay lifetime by sampling in a first time bin after illumination and a second time bin after illumination, where the second time bin is subsequent to the first time bin. The ratio between a number of photons detected in the first time bin and a number of photons detected in the second time bin may provide the bin ratio value. Different fluorescent dyes conjugated to different NAA recognizers may exhibit different fluorescence decay characteristics, resulting in different bin ratio values that can be used to distinguish between recognizers.

With continued reference to FIG. 13, six distinct dye classes are represented as nested ellipses overlaid on the distribution. Each set of nested ellipses may represent one standard deviation of a normal distribution associated with each dye class in the Gaussian Mixture Model classifier. The ellipses labeled LIV are positioned in a lower left region of the plot at approximately 0.2 bin ratio and 4.25 to 4.5 log intensity. The LIV dye class may correspond to a recognizer that binds to leucine, isoleucine, and valine amino acid residues. The ellipses labeled AS are positioned in a center-left region at approximately 0.3 bin ratio and 4.5 to 4.75 log intensity. The AS dye class may correspond to a recognizer that binds to alanine and serine amino acid residues.

As further shown in FIG. 13, the ellipses labeled R are positioned in an upper-center region at approximately 0.4 bin ratio and 5.0 to 5.25 log intensity. The R dye class may correspond to a recognizer that binds to arginine amino acid residues. The ellipses labeled NQ are positioned in a center region at approximately 0.5 bin ratio and 4.5 to 4.75 log intensity. The NQ dye class may correspond to a recognizer that binds to asparagine and glutamine amino acid residues. The ellipses labeled FYW are positioned in an upper-right region at approximately 0.6 bin ratio and 4.75 to 5.0 log intensity. The FYW dye class may correspond to a recognizer that binds to phenylalanine, tyrosine, and tryptophan amino acid residues. The ellipses labeled DE are positioned in a lower-right region at approximately 0.65 bin ratio and 4.25 to 4.5 log intensity. The DE dye class may correspond to a recognizer that binds to aspartic acid and glutamic acid amino acid residues.

In some embodiments, the Gaussian Mixture Model classifier illustrated in FIG. 13 may be trained on-the-fly using pulses sampled from across a chip of a sequencing device and throughout a sequencing run. After the Gaussian Mixture Model is fit, each cluster may be associated with one of the dyes in a sequencing kit based on pre-calculated cross-run averages of measured intensities and bin ratios for each dye. The distribution of pulse bin ratios and intensities for each dye class may overlap to some extent, as shown by the proximity of certain ellipses in FIG. 13. Consequently, even nominally pure recognition segments consisting of pulses for a single dye may have non-zero purity values for other dyes due to the overlapping distributions. The Gaussian Mixture Model classifier may be used to determine which recognizer is associated with each recognition segment by measuring how consistent a distribution of pulse intensities and bin ratios within the recognition segment are with each of the dye classes in the model.

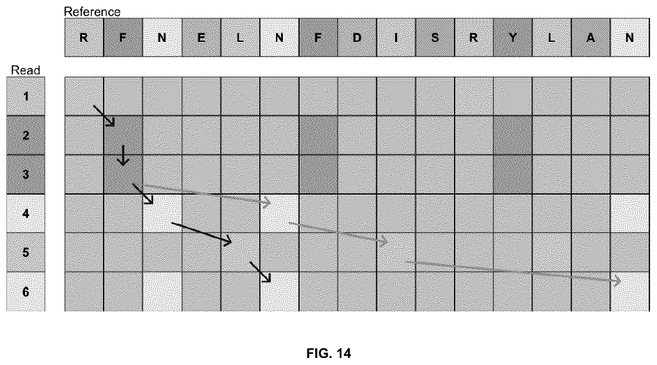

FIG. 14 illustrates an example alignment between a read and a reference peptide sequence, according to some embodiments of the technology described herein. Referring to FIG. 14, the reference peptide sequence is shown along a top row with letters R, F, N, E, L, N, F, D, I, S, R, Y, L, A, N corresponding to amino acid residues. The read is shown along a left column with rows numbered 1 through 6 representing recognition segments. The grid illustrates an alignment process where colored squares indicate positions where a recognizer assigned to a recognition segment matches an expected recognizer for an amino acid residue in the reference, while grey squares indicate positions where the recognizers do not match.

As illustrated in FIG. 14, a recognizer 1 corresponds to the R amino acid residue in the reference peptide sequence. A recognizer 2 corresponds to the F amino acid positions in the reference peptide sequence. A recognizer 3 corresponds to the N amino acid positions in the reference peptide sequence. The recognizer 4 corresponds to the D amino acid residue. The recognizer 5 corresponds to the S amino acid residue. The recognizer 6 corresponds to the Y amino acid residue. Amino acid residues in the reference peptide sequence that do not have a matching recognizer, such as E, L, I, and A, may be skipped by the alignment module 108. As a result, these amino acid residues will not be aligned with any recognition segments in the read.

With continued reference to FIG. 14, a black arrow trajectory shows a most likely alignment path through the grid. The first arrow trajectory represents an alignment in which recognition segments are matched to amino acid residues in the reference peptide sequence based on recognizer compatibility and alignment scoring. A second arrow trajectory shows a second valid alignment path involving multiple deletions. The second arrow trajectory represents an alternative alignment in which certain visible states in the reference peptide sequence are not aligned with any recognition segment in the read, resulting in deletion penalties being applied to the alignment score.

In some embodiments, the alignment shown in FIG. 14 demonstrates that multiple adjacent recognition segments may align to a same reference state. For example, recognition segments 2 and 3 in the read may both correspond to a first F amino acid residue in the reference peptide sequence, which can occur when a recognition segment is over-split during the segmentation process performed by the pulse segmentation module 104. The alignment module 108 may be configured to permit multiple adjacent recognition segments to align to a same state of the reference peptide sequence to ensure that a recognition segment that was erroneously split into multiple recognition segments can still align.

As further shown in FIG. 14, for an alignment trajectory to be valid, the trajectory may be required to align all recognition segments in the read, but the trajectory does not need to reach an end of the reference peptide sequence. The alignment module 108 may be configured to calculate a final alignment score S_{alignment} as:

$${S}_{alignment} = {\sum }_{i}^{{N}_{RSs}} {w}_{RS}^{( i )} \times ( {s}_{match}^{( i )} + {s}_{gap}^{( i )} ) + {\sum }_{j}^{{N}_{deletions}} {s}_{deletion}^{( j )}$$

where w_{RS}⁽ⁱ⁾ is a recognition segment weight for the i-th recognition segment, s_{match}⁽ⁱ⁾ is a match score for the i-th recognition segment, s_{gap}⁽ⁱ⁾ is a gap score for the i-th recognition segment, and s_{deletion}⁽ⁱ⁾ is a deletion score for the j-th deleted state. The alignment score calculation sums contributions from match scores and gap scores weighted by recognition segment weights, plus deletion penalties for any skipped states from the reference peptide sequence.

In some embodiments, the alignment module 108 may be configured to assign recognition segment weights (w_{RS}) equal to 1/n where n is a number of adjacent recognition segments in the read that have been labeled with a same recognizer. This weighting approach may address over-splitting bias that can occur when a state is over-split, which may increase a total number of recognition segments in the read and thereby increase a maximum possible alignment score. For example, if a recognition segment recognizer sequence is ABBCB, then the recognition segment weights are 1, ½, ½, 1, 1. By applying recognition segment weights, the alignment module 108 may reduce the effect of over-split recognition segments on alignment scores, which may reduce bias introduced by the over-splitting.

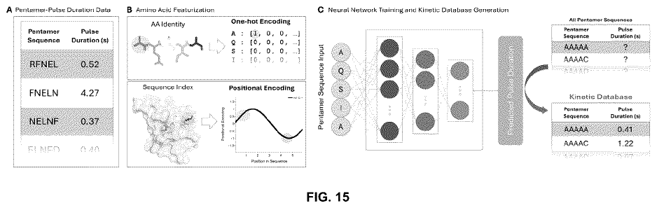

FIG. 15 illustrates an architectural overview of a neural network used to generate a kinetic database storing pulse duration predictions for amino acid motifs, according to some embodiments of the technology described herein. Referring to FIG. 15, the figure is organized into three panels labeled A, B, and C that depict different aspects of the neural network architecture and training process.

Panel A of FIG. 15 shows pentamer-pulse duration data that serves as input for training, validation, and testing of the neural network. Panel A displays a table with two columns: a pentamer sequence column and a pulse duration column measured in seconds. The table displays example pentamer sequences including RFNEL with a pulse duration of 0.52 seconds, FNELN with a pulse duration of 4.27 seconds, NELNF with a pulse duration of 0.37 seconds, and ELNFD with a pulse duration of 0.40 seconds. The pentamer sequences represent amino acid motifs of five residues in length, where each motif comprises an N-terminal residue and four subsequent downstream residues in a direction from the N-terminus towards the C-terminus. The pulse duration values represent empirically measured or predicted durations of light pulses generated during binding interactions between fluorescently tagged NAA recognizers and the corresponding amino acid motifs during sequencing.

With continued reference to FIG. 15, Panel B illustrates amino acid featurization comprising two components: amino acid identity encoding and sequence index encoding. The amino acid identity component shows a molecular structure representation with colored atoms and corresponding one-hot encoding vectors. In the one-hot encoding representation, each amino acid is encoded as a binary vector where a single position has a value of 1 and all other positions have values of 0. For example, Panel B shows that amino acid A may be encoded as [1, 0, 0, . . . ], amino acid Q may be encoded as [0, 0, 0, . . . ], amino acid S may be encoded as [0, 0, 0, . . . ], and amino acid I may be encoded as [0, 0, 0, . . . ]. The one-hot encoding captures amino acid identity information by providing a unique binary representation for each of the 20 canonical amino acids.

As further shown in Panel B of FIG. 15, the sequence index component shows a protein surface representation with a bound peptide and a corresponding positional encoding graph. The positional encoding graph plots positional encoding values against position in sequence from positions 1 through 5. The sinusoidal positional encoding captures position information for each amino acid within the pentamer motif using sine and cosine functions at different frequencies. The positional encoding may be defined elementwise as PE(pos, 2i)=sin (pos/10000^{(2i/D}_{model}⁾) and PE(pos, 2i+1)=cos (pos/10000^{(2i/D}_{model}⁾), where pos is a position within the sequence and i indexes a dimension. The positional context captured by the sinusoidal encoding may be based on a recognizer binding mode, where an amino acid at a first position in the peptide (shown as red sticks and spheres in Panel B) may contribute more to an overall pulse duration compared to a same amino acid identity at position five (shown as blue sticks and spheres in Panel B) due to stronger intermolecular contacts at positions closer to the N-terminus.

Referring to Panel C of FIG. 15, the neural network training and kinetic database generation process is depicted. The neural network architecture shows a pentamer sequence input layer with nodes labeled A, Q, S, I, and A, representing amino acid residues at each of the five positions in the pentamer motif. The input layer receives a combination of one-hot encoded amino acid identities and sinusoidal positional encodings generated for the pentamer motif. The input layer is connected through multiple hidden layers represented by nodes arranged in successive columns. The hidden layers may comprise fully connected layers with gradually reduced dimensionality, such as dimensions of 128→64→16, to enhance computational efficiency and parameter regularization. The hidden layers may be normalized using batch normalization and regularized with a dropout rate. ReLU activation functions may be employed in the hidden layers to improve training stability and accelerate convergence.

With continued reference to Panel C of FIG. 15, the neural network produces a predicted pulse duration output from an output layer. The output layer may produce a single-value regression estimate representing a predicted pulse duration for the input pentamer motif. The predicted pulse duration output feeds into generation of a kinetic database. Panel C shows a first table representing all pentamer sequences with unknown pulse durations indicated by question marks for sequences AAAAA, AAAAC, and AAAAD. The trained neural network may be applied to predict pulse durations for pentamer motifs that have not been empirically measured. Panel C shows a second table representing the kinetic database with predicted pulse durations, where AAAAA has a pulse duration of 0.41 seconds and AAAAC has a pulse duration of 1.22 seconds. An arrow indicates a flow from the neural network predictions to populate the kinetic database.

In some embodiments, the kinetic database may comprise 2,080,000 unique pentameric sequences. The 2,080,000 unique pentameric sequences may be calculated as 13× 20⁴, covering all combinations of 13 N-terminally recognized amino acids at a first position with 20 canonical amino acids in each of four downstream positions. The 13 N-terminally recognized amino acids may correspond to amino acids for which fluorescently tagged NAA recognizers are available in a sequencing kit. For example, a sequencing kit may include recognizers for LIV (leucine, isoleucine, valine), FYW (phenylalanine, tyrosine, tryptophan), R (arginine), AS (alanine, serine), DE (aspartic acid, glutamic acid), and NQ (asparagine, glutamine), providing recognition capability for 13 of the 20 canonical amino acids. The four downstream positions may each contain any of the 20 canonical amino acids, resulting in 20⁴=160,000 possible combinations for each of the 13 N-terminally recognized amino acids at the first position.

In some embodiments, the kinetic database may include empirical pulse duration values when previously measured for a corresponding pentameric motif during sequencing runs. When a pulse duration has not been previously measured for a particular pentameric motif, a predicted value from the trained neural network may be stored in the kinetic database. This approach allows generation of a comprehensive kinetic database that pairs each of the 2,080,000 pentameric sequences with a corresponding pulse duration value for use in scoring alignments against any pentameric sequence motif during downstream alignment and variant detection processes.

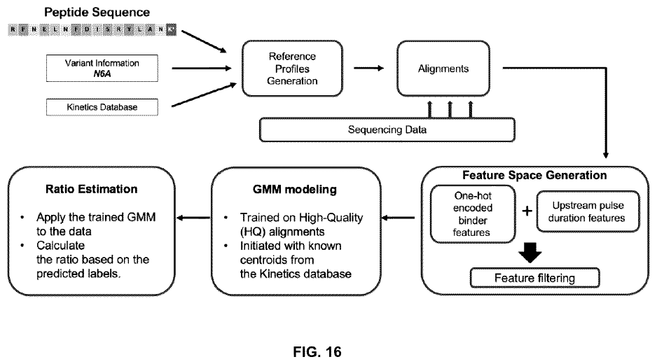

FIG. 16 illustrates a flowchart for a variant detection workflow using next-generation protein sequencing data, according to some embodiments of the technology described herein. Referring to FIG. 16, the workflow begins with multiple inputs that are provided to initiate the variant detection process. The inputs include a peptide sequence representing a reference peptide for which variants are to be detected, variant information identifying a substitution such as N6A (asparagine to alanine at position 6), and a kinetics database containing expected pulse duration values for amino acid motifs. The kinetics database may correspond to the reference alignment data 114 described herein with reference to FIGS. 1A-1C, which pairs amino acid motifs with corresponding pulse duration values.

As illustrated in FIG. 16, the inputs feed into a reference profiles generation step. The reference profiles generation step may be configured to produce reference profiles for peptide variants using the peptide sequence, variant information, and kinetics database. The reference profiles may comprise expected peptide states of the variants and upstream residues, along with corresponding pulse duration profiles retrieved from the kinetics database. In some embodiments, the reference profiles generation step may be configured to retrieve expected pulse durations for amino acid motifs spanning from a variant site up to a particular number of residues upstream (e.g., four residues upstream). The reference profiles may be used to segment clusters of pulse durations and assign clusters to variants in subsequent processing steps.

With continued reference to FIG. 16, the reference profiles are provided to an alignments step. The alignments step also receives sequencing data as input. The sequencing data may comprise reads generated from sequencing a sample containing one or more variants of the peptide. The alignments step may be configured to align sequencing reads to the reference profiles for each variant. In some embodiments, the alignments step may use a minimum alignment score threshold for reads to be used in aggregating positional kinetics. The minimum alignment score threshold may be 3.75. Reads with alignment scores at or above the minimum alignment score threshold of 3.75 may be used in subsequent processing to reduce the influence of ambiguous reads on variant detection results.

As further shown in FIG. 16, the workflow proceeds to a feature space generation step. The feature space generation step may be configured to construct a multidimensional feature space by combining one-hot encoded binder features with upstream pulse duration features. The one-hot encoded binder features may comprise binary representations of recognizers assigned to recognition segments at respective positions in the aligned reads. The upstream pulse duration features may comprise pulse duration measurements extracted from recognition segments aligned to positions upstream of the variant site. In some embodiments, the feature space generation step may integrate aggregated positional kinetics spanning from the variant site up to four residues upstream with the one-hot encoded binder features. These combined inputs may capture recognizer read variation and the context influencing pulse duration for variant discrimination.

With continued reference to FIG. 16, the combined features undergo a feature filtering step. The feature filtering step may be configured to select features that are relevant for variant discrimination. In some embodiments, the feature filtering step may select a single position showing a largest pulse duration difference between variant peptides as a primary feature for clustering. The feature filtering step may reduce dimensionality of the feature space while retaining features that provide discriminative power for distinguishing between variant populations.

As illustrated in FIG. 16, the filtered features are provided to a Gaussian Mixture Model (GMM) modeling step. The GMM modeling step may be configured to train a two-component GMM on high-quality alignments. The GMM may be initiated with known centroids from the kinetics database. The known centroids may correspond to expected kinetic profiles for the variant peptides, including expected pulse durations retrieved from the kinetics database for amino acid motifs at the variant site and upstream positions. By initiating the GMM with known centroids derived from the kinetics database, the GMM modeling step may guide the clustering process to identify variant populations based on expected kinetic differences between the variants.

As further shown in FIG. 16, the trained GMM is applied in a ratio estimation step. The ratio estimation step may be configured to apply the trained GMM to the data to calculate a ratio of variant populations based on predicted labels. Applying the trained GMM to the feature sets may yield predicted population identities for all data points, with each data point assigned to one of the two variant populations based on the GMM clustering. The ratio estimation step may calculate the ratio of variant populations from the GMM populations by determining a proportion of data points assigned to each variant population. The workflow outputs variant population ratio estimates, providing quantification of the relative abundance of each variant in the sample.

In some embodiments, the variant detection workflow illustrated in FIG. 16 may accept raw sequencing data from a binary mixture along with a reference sequence and variant information as inputs, and may produce variant population ratio estimates as output. The integration of kinetic features with recognizer encoding may enable discrimination between peptide variants, including variants where both amino acids lack direct recognizers. The use of the minimum alignment score threshold of 3.75 for reads used in aggregating positional kinetics may reduce noise from ambiguous alignments and improve accuracy of variant quantification.

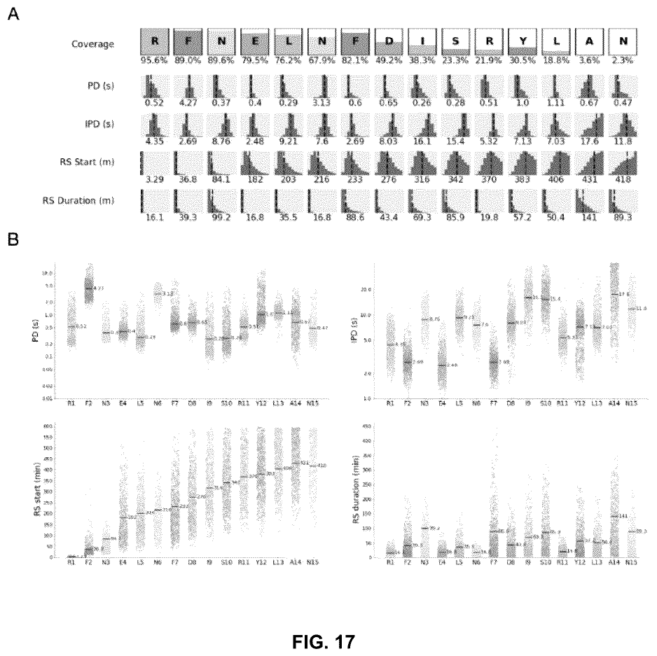

FIG. 17 illustrates kinetic properties of a pure N6 peptide across multiple amino acid positions in the peptide sequence, according to some embodiments of the technology described herein. Referring to FIG. 17, the figure is organized into two parts labeled A and B that depict different representations of kinetic property measurements obtained from aligned reads for the N6 peptide variant.

Part A of FIG. 17 shows a kinetics summary of aligned reads displayed as a table with rows corresponding to different kinetic properties and columns corresponding to amino acid positions in the peptide sequence. The rows of the table include Coverage, PD (pulse duration in seconds), IPD (inter-pulse duration in seconds), RS Start (recognition segment start time in minutes), and RS Duration (recognition segment duration in minutes). The columns correspond to amino acid positions labeled R, F, N, E, L, N, F, D, I, S, R, Y, L, A, and N from left to right, representing the sequence of amino acid residues in the N6 peptide variant. Each cell in the table contains a small histogram distribution and a numerical value representing a mean for that property at that position.

With continued reference to Part A of FIG. 17, the Coverage row displays coverage values for each amino acid position. The coverage may represent a fraction of aligned reads that contain a recognition segment aligned to that state. In some embodiments, the coverage may indicate a fraction of reads in which that state was not deleted in alignment. The coverage values may decrease from a first position to a final position in the peptide sequence. For example, the coverage values may decrease from 95.6% at the first position (R) to 2.3% at the final position (N). The decrease in coverage for states deeper into the peptide may result from sequencing limitations, where recognition segments corresponding to amino acid residues at later positions in the peptide sequence may be less frequently observed in aligned reads.

As further shown in Part A of FIG. 17, the PD row displays pulse duration measurements in seconds for each amino acid position. The pulse duration values may vary across positions based on kinetic properties of recognizer-peptide interactions at each position. The IPD row displays inter-pulse duration measurements in seconds for each amino acid position. The inter-pulse duration values may reflect association kinetics of recognizer-peptide complexes at each position. The RS Start row displays recognition segment start times in minutes for each amino acid position. The recognition segment start times may indicate when recognition segments corresponding to each amino acid position begin during a sequencing run. The RS Duration row displays recognition segment duration measurements in minutes for each amino acid position. The recognition segment duration values may indicate how long recognition segments corresponding to each amino acid position persist during sequencing.

Referring to Part B of FIG. 17, four scatter plots with overlaid mean values are shown for each amino acid position in the peptide sequence. The scatter plots provide per-read distributions of the various kinetic properties across the amino acid positions R1 through N15.

A first scatter plot in Part B of FIG. 17, positioned in an upper left region, displays pulse duration in seconds on a logarithmic y-axis ranging from 0.01 to 10.0 seconds across the amino acid positions on the x-axis. Each scatter plot shows individual data points as dots corresponding to each amino acid position, with horizontal lines indicating mean values for each position. The pulse duration scatter plot may illustrate variation in pulse duration measurements across individual reads for each amino acid position, with the mean pulse duration values providing a summary statistic for each position.

A second scatter plot in Part B of FIG. 17, positioned in an upper right region, displays inter-pulse duration in seconds on a y-axis ranging from 1.0 to 20.0 seconds across the amino acid positions. The inter-pulse duration scatter plot may illustrate variation in inter-pulse duration measurements across individual reads for each amino acid position. The inter-pulse duration values may be governed by association kinetics of recognizer-peptide complexes, with variation across positions reflecting differences in binding kinetics for different amino acid residues.

A third scatter plot in Part B of FIG. 17, positioned in a lower left region, displays recognition segment start time in minutes on a y-axis ranging from 0 to 600 minutes across the amino acid positions. The recognition segment start time scatter plot may illustrate when recognition segments corresponding to each amino acid position begin during a sequencing run. Recognition segment start times may increase for amino acid positions deeper into the peptide sequence, as aminopeptidases sequentially cleave N-terminal amino acids to expose successive amino acids for recognition.

A fourth scatter plot in Part B of FIG. 17, positioned in a lower right region, displays recognition segment duration in minutes on a y-axis ranging from 0 to 450 minutes across the amino acid positions. The recognition segment duration scatter plot may illustrate how long recognition segments corresponding to each amino acid position persist during sequencing. The recognition segment duration values may reflect a time during which a particular amino acid residue is exposed at the N-terminus and available for binding by fluorescently tagged NAA recognizers before being cleaved by aminopeptidases.

In some embodiments, the kinetic properties illustrated in FIG. 17 may be used to characterize peptide states in pure form. The set of reads that align to a reference peptide sequence may be used to calculate kinetic properties of various peptide states in that reference, including mean pulse duration, mean inter-pulse duration, recognition segment start time, and recognition segment duration. The kinetic properties may provide information that can be used for variant detection by comparing observed kinetic properties from sequencing data to expected kinetic properties for different variant peptide sequences.

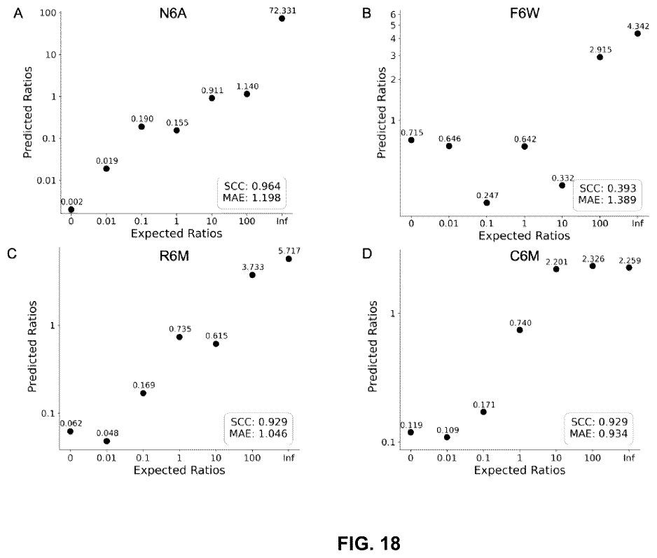

FIG. 18 illustrates scatter plots comparing predicted ratios against expected ratios for four variant titration datasets, according to some embodiments of the technology described herein. Referring to FIG. 18, the figure comprises four panels labeled A, B, C, and D, each corresponding to a different variant pair analyzed using the variant detection workflow described herein. Each scatter plot has an x-axis showing expected ratios between two variant peptides in log scale and a y-axis showing predicted ratios in log scale. The scatter plots display data points with numerical values labeled above each point indicating the estimated ratio at that titration level.

In some embodiments, performance evaluation of the variant detection workflow may use a Mean Absolute Error (MAE) calculated in log scale between estimated ratios and expected ratios. The MAE may be calculated as:

$$MAE = \frac{1}{n} {\sum }_{i = 1}^{n} \vert {log}_{10} ( {p}_{i} ) - {log}_{10} ( {r}_{i} ) \vert$$

where n is a total number of data points, pᵢ is a predicted ratio of data point i, and rᵢ is an expected ratio of data point i. The MAE describes a deviation between predicted values and expected values in log space. A value of 1 in MAE indicates that an estimation ratio differs from an expected ratio by a factor of 10. In some embodiments, maintaining predictions within a factor of 10 of expected values may be within expected variability for peptide population predictions given inherent noise in biological data, model limitations, and peptide concentration error.

In some embodiments, performance evaluation may use a Spearman's correlation coefficient (SCC) to assess correlation between expected and estimated variant ratios. The SCC may provide a measure of how well the predicted ratios track the expected ratios across the range of titration levels tested.

With continued reference to FIG. 18, Panel A corresponds to variant N6A, which represents an asparagine to alanine substitution at the sixth position of the peptide sequence. Panel A displays data points with values including 0.002, 0.019, 0.190, 0.155, 0.911, 1.140, and 72.331 at different titration levels. The scatter plot for variant N6A demonstrates a diagonal trend indicative of correlation between estimated and expected ratios. Panel A shows an SCC of 96.4% and an MAE of 1.198 in a lower right corner of the plot. The N6A variant represents a visible-to-visible variant where both asparagine and alanine amino acid residues have corresponding recognizers.

As further shown in FIG. 18, Panel B corresponds to variant F6W, which represents a phenylalanine to tryptophan substitution at the sixth position. Panel B displays data points with values including 0.247, 0.715, 0.646, 0.642, 0.332, 2.915, and 4.342 at different titration levels. Panel B shows an SCC of 39.3% and an MAE of 1.389. The F6W variant represents a case where both peptides share a same recognizer at the variant position, as phenylalanine and tryptophan are both recognized by the FYW recognizer. The lower SCC for the F6W variant may result from identical recognizer features limiting alignment and clustering discriminative power. However, the kinetic features may still capture overall kinetic differences between the variants, providing a reasonable approximation of expected ratios for this challenging variant type.

Referring to Panel C of FIG. 18, the scatter plot corresponds to variant R6M, which represents an arginine to methionine substitution at the sixth position. Panel C displays data points with values including 0.062, 0.048, 0.169, 0.735, 0.615, 3.733, and 5.717 at different titration levels. Panel C shows an SCC of 92.9% and an MAE of 1.046. The R6M variant represents a visible-to-invisible variant where arginine has a corresponding recognizer but methionine does not have a corresponding recognizer. The predictions for the R6M variant generally agree with expected ratios, demonstrating that the variant detection workflow can differentiate populations based on upstream changes in kinetic features even when one variant lacks a direct recognizer at the variant position.

As illustrated in Panel D of FIG. 18, the scatter plot corresponds to variant C6M, which represents a cysteine to methionine substitution at the sixth position. Panel D displays data points with values including 0.119, 0.109, 0.171, 0.740, 2.201, 2.326, and 2.259 at different titration levels. Panel D shows an SCC of 92.9% and an MAE of 0.934. The C6M variant represents an invisible-to-invisible variant where neither cysteine nor methionine has a corresponding recognizer. By leveraging kinetic features at upstream positions, the variant detection workflow may capture a general trend of expected ratios. The MAE of 0.934 for the C6M variant indicates that predictions are within a factor of approximately 10 of expected values on average.

In some embodiments, the variant detection workflow may detect variants down to 2 μM on-chip concentration. For example, a lowest peptide input of 1 nM in a 1:100 dilution mixture may result in detection of variants at approximately 2 μM on-chip concentration. The ability to detect variants at picomolar concentrations may enable analysis of samples with low abundance variants or limited sample quantities.

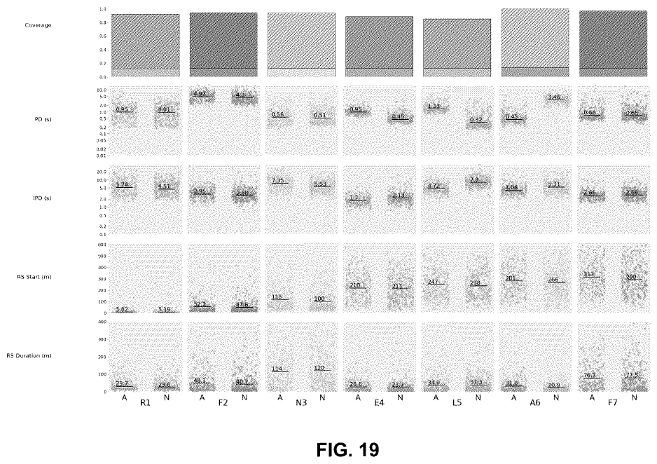

FIG. 19 illustrates a multi-panel visualization showing kinetic properties of seven peptide variants across multiple amino acid positions in the peptide sequence, according to some embodiments of the technology described herein. Referring to FIG. 19, the visualization is organized into five horizontal rows corresponding to different kinetic property measurements, with columns representing amino acid positions labeled R1, F2, N3, E4, L5, A6, and F7. Each variant position shows paired conditions labeled A and N for comparison between different experimental conditions or variant types.

As illustrated in FIG. 19, a first row displays Coverage measurements as bar charts with diagonal hatching patterns in different colors for each variant pair. The coverage values may approach 1.0 for earlier positions in the peptide sequence and may decrease for later positions. The coverage measurements may indicate a fraction of aligned reads that contain a recognition segment aligned to each amino acid position, with higher coverage at N-terminal positions and lower coverage at positions deeper into the peptide sequence.

With continued reference to FIG. 19, a second row displays PD (pulse duration) measurements in seconds as scatter plots on a logarithmic scale ranging from approximately 0.01 to 10.0 seconds. Numerical values are displayed above each scatter plot indicating mean pulse durations for each condition. The pulse duration scatter plots may illustrate variation in pulse duration measurements across individual reads for each amino acid position under each condition. The pulse duration values may exhibit distinct separation not only at variant sites but also at upstream residues, consistent with expected kinetic effects on upstream positions. The kinetic effects on upstream positions may span from the variant site up to four residues upstream, arising from interactions between NAA recognizers and downstream positions relative to the N-terminal amino acid being recognized.

As further shown in FIG. 19, a third row displays IPD (inter-pulse duration) measurements in seconds as scatter plots on a logarithmic scale ranging from approximately 0.1 to 20.0 seconds. Mean values are shown above each scatter plot. The inter-pulse duration measurements may reflect association kinetics of recognizer-peptide complexes at each position, with variation across positions reflecting differences in binding kinetics for different amino acid residues under different conditions.

Referring to a fourth row of FIG. 19, RS Start (recognition segment start time) measurements are presented as scatter plots showing values in minutes, with values ranging from 0 to approximately 600 minutes. Mean values are displayed above each scatter plot. The recognition segment start time measurements may indicate when recognition segments corresponding to each amino acid position begin during a sequencing run. Recognition segment start times may increase for amino acid positions deeper into the peptide sequence as aminopeptidases sequentially cleave N-terminal amino acids to expose successive amino acids for recognition.

As illustrated in a fifth row of FIG. 19, RS Duration (recognition segment duration) measurements are shown as scatter plots with values in minutes ranging from 0 to approximately 400 minutes. Mean values are indicated above each scatter plot. The recognition segment duration measurements may indicate how long recognition segments corresponding to each amino acid position persist during sequencing.

In some embodiments, the paired A and N conditions within each amino acid position may allow comparison of kinetic properties between different variant peptides across all seven variant positions shown in FIG. 19. The distinct separation in pulse duration values at variant sites and upstream residues may provide kinetic signatures that can be used to discriminate between variant populations. The kinetic effects observed at upstream positions may result from the influence of downstream amino acid residues on recognizer binding kinetics, where an amino acid substitution at a variant site may alter pulse duration measurements at positions up to four residues upstream of the variant site. This upstream kinetic effect may enable variant detection even when the variant amino acid residue itself lacks a direct recognizer, as changes in kinetic properties at upstream positions may provide discriminative features for clustering reads into variant populations.

### Example Techniques for Training Classification Model for Recognizer Assignment

In some embodiments, the classification model 106A (e.g., classification model 306 described herein with reference to FIG. 3A) (also referred to herein as a “dye caller”) may be trained (e.g., as part of block 304 described herein with reference to FIG. 3A) on a run-by-run basis. Training of the classification model 106A may comprise the following steps: (1) calculating per-aperture intensity correction factors before calling the dye caller fitting routine; (2) collecting a random sample of pulses (e.g., 100,000 pulses, 200,000 pulses, 300,000 pulses, or another suitable number of pulses) from across the whole run; (3) calculating an absolute intensity of the run by performing expectation-maximization (EM) on a Gaussian Mixture Model (GMM) with fixed relative center locations and fixed variance; (4) collecting a biased sample of pulses (e.g., 100,000 pulses, 200,000 pulses, 300,000 pulses, or another suitable number of pulses) using an initial guess GMM with fixed centers determined by the previous step and fixed variance, where Metropolis-Hastings sampling is used to probabilistically accept or reject pulses based on how they change an entropy of relative dye fractions as determined by the guess GMM; (5) fitting a GMM comprising one component per dye plus an extra junk component using EM, with priors placed on centers and variances of components corresponding to each dye but not the junk center; (6) removing any dye component whose weight is less than a threshold percentage (e.g., a percentage in the range 1%-10% such as 5%) of the dye component with the highest weight, as such components may be considered not found; (7) re-fitting the remaining dye components plus the junk component using EM without priors; and (8) setting centers of any missing dyes to values in a reagents database and constructing the final classification model.

### GMM EM Derivation

In some embodiments, the classification model 106A may be trained using an expectation-maximization (EM) algorithm applied to a Gaussian Mixture Model (GMM). For a standard two-dimensional GMM with K components and N observations (e.g., pulses), the likelihood may be given by:

$$p ( \{ {x}_{i} \} \vert \{ {w}_{j} , {μ}_{j} , {\sum }_{j} \} ) = {\prod }_{i = 1}^{N} {\sum }_{j = 1}^{K} \frac{{w}_{j}}{2 π \sqrt{\vert {\sum }_{j} \vert}} exp [ - \frac{1}{2} {( {x}_{i} - {μ}_{j} )}^{T} {\sum }_{j}^{- 1} ( {x}_{i} - {μ}_{j} ) ]$$

where xᵢ is the location of observation i in two-dimensional space (e.g., bin ratio versus log (intensity)), wⱼ is the weight of component j, μⱼ is the center of component j, and Σⱼ is the covariance matrix of component j. Because the likelihood is a product of sums, the log-likelihood (a more numerically convenient target for direct optimization) becomes a sum of logs of sums:

$$log ( p ( \{ {x}_{i} \} \vert \{ {w}_{j} , {μ}_{j} , {\sum }_{j} \} ) ) = {\sum }_{i = 1}^{N} log ( {\sum }_{j = 1}^{K} \frac{{w}_{j}}{2 π \sqrt{\vert {\sum }_{j} \vert}} exp [ - \frac{1}{2} {( {x}_{i} - {μ}_{j} )}^{T} {\sum }_{j}^{- 1} ( {x}_{i} - {μ}_{j} ) ] )$$

In some embodiments, the parameters of the GMM (wⱼ, μⱼ, and Σⱼ) may not be separable. However, if each data point were known to belong to a particular component of the GMM, it would be possible to rewrite the log-likelihood as a sum of sums:

$$log ( p ( \{ {x}_{i} , {z}_{ij} \} \vert \{ {μ}_{j} , {\sum }_{j} \} ) ) = {\sum }_{i = 1}^{N} {\sum }_{j = 1}^{K} {z}_{i j} [ log ( \frac{1}{2 π \sqrt{\vert {\sum }_{j} \vert}} ) - \frac{1}{2} {( {x}_{i} - {μ}_{j} )}^{T} {\sum }_{j}^{- 1} ( {x}_{i} - {μ}_{j} ) ]$$

where zᵢⱼ are the latent variables describing which component corresponds to each sample. Specifically, for a given pulse i, only one of zᵢ₁, zᵢ₂, . . . , z_{iK} is equal to 1, with all others being equal to 0.

In some embodiments, the EM algorithm may replace zᵢⱼ with a continuous variable γᵢⱼ, allowing analytical solution for optimal values of μⱼ and Σⱼ that maximize the log likelihood. This is a valid application of Jensen's inequality if γᵢⱼ is chosen to be the expectation value of the latent variable zᵢⱼ for a given set of parameters. The variables γᵢⱼ, also known as the responsibilities, may be calculated as:

$${γ}_{ij} = \frac{\frac{{w}_{j}}{2 π \sqrt{\vert {\sum }_{j} \vert}} exp [ - \frac{1}{2} {( {x}_{i} - {μ}_{j} )}^{T} {\sum }_{j}^{- 1} ( {x}_{i} - {μ}_{j} ) ]}{{\sum }_{{j}^{′} = 1}^{K} \frac{{w}_{{j}^{′}}}{2 π \sqrt{\vert {\sum }_{{j}^{′}} \vert}} exp [ - \frac{1}{2} {( {x}_{i} - {μ}_{{j}^{l}} )}^{T} {\sum }_{{j}^{′}}^{- 1} ( {x}_{i} - {μ}_{{j}^{′}} ) ]}$$

This leads to an auxiliary optimization target that is more amenable to direct optimization:

$$A ( \{ {μ}_{j} , {\sum }_{j} \} \vert \{ {μ}_{j}^{( t )} , {\sum }_{j}^{( t )} \} ) = {\sum }_{i = 1}^{N} {\sum }_{j = 1}^{K} {γ}_{l j} [ log ( \frac{1}{2 π \sqrt{\vert {\sum }_{j} \vert}} ) - \frac{1}{2} {( {x}_{i} - {μ}_{j} )}^{T} {\sum }_{j}^{- 1} ( {x}_{i} - {μ}_{j} ) ]$$

where μⱼ^{(t)} and Σⱼ^{(t)} are the parameters that were used to calculate γᵢⱼ from the previous iteration of the algorithm. This results in an iterative optimization algorithm: (1) guess initial parameters wⱼ, μⱼ, and Σⱼ; (2) E-step: calculate the responsibilities γᵢⱼ; (3) update wⱼ according to γᵢⱼ by summing γᵢⱼ over i and normalizing so the weights add to 1; (4) M-step: maximize the auxiliary function with respect to μⱼ and then Σⱼ; and (5) return to step 2 and repeat until convergence. To determine convergence, either the parameters themselves or the log-likelihood may be monitored.

### Incorporating Per-Pulse Uncertainties

In some embodiments, the classification model 106A may differ from a standard GMM in that it accounts for per-pulse uncertainties in measured bin ratios and log (intensities). This may be accomplished by augmenting the covariance matrix Σⱼ with a per-pulse variance Δᵢ that is a function of both the pulse duration and the background noise reported by the pulse caller. The likelihood used by the classification model 106A may be:

$$p ( \{ {x}_{i} , {Δ}_{i} \} \vert \{ {w}_{j} , {μ}_{j} , {\sum }_{j} \} ) = {\prod }_{i = 1}^{N} {\sum }_{j = 1}^{K} \frac{{w}_{j}}{2 π \sqrt{\vert {\sum }_{j} + {Δ}_{i} \vert}} exp [ - \frac{1}{2} {( {x}_{i} - {μ}_{j} )}^{T} {( {\sum }_{j} + {Δ}_{i} )}^{- 1} ( {x}_{i} - {μ}_{j} ) ]$$

Each instance of Σⱼ has been replaced with Σⱼ+Δᵢ. This has a moderate impact on the E-step, but a larger impact on the M-step. The E-step becomes:

$${γ}_{ij} = \frac{\frac{{w}_{j}}{2 π \sqrt{\vert {\sum }_{j} + {Δ}_{i} \vert}} exp [ - \frac{1}{2} {( {x}_{i} - {μ}_{j} )}^{T} {( {\sum }_{j} + {Δ}_{i} )}^{- 1} ( {x}_{i} - {μ}_{j} ) ]}{{\sum }_{{j}^{′} = 1}^{K} \frac{{w}_{{j}^{′}}}{2 π \sqrt{\vert {\sum }_{{j}^{′}} + {Δ}_{i} \vert}} exp [ - \frac{1}{2} {( {x}_{i} - {μ}_{{j}^{′}} )}^{T} {( {\sum }_{{j}^{′}} + {Δ}_{i} )}^{- 1} ( {x}_{i} - {μ}_{{j}^{′}} ) ]}$$

And the auxiliary function for the M-step becomes:

$$A ( \{ {μ}_{j} , {\sum }_{j} \} \vert \{ {μ}_{j}^{( t )} , {\sum }_{j}^{( t )} \} ) = {\sum }_{i = 1}^{N} {\sum }_{j = 1}^{K} {γ}_{ij} [ log ( \frac{1}{2 π \sqrt{\vert {\sum }_{j} + {Δ}_{i} \vert}} ) - \frac{1}{2} {( {x}_{i} - {μ}_{j} )}^{T} {( {\sum }_{j} + {Δ}_{i} )}^{- 1} ( {x}_{i} - {μ}_{j} ) ]$$

It remains possible to maximize the auxiliary function with respect to μⱼ analytically, but it is no longer possible to analytically maximize the auxiliary function with respect to Σⱼ. Instead, Σⱼ may be solved for using numerical methods.

### Incorporating Priors on Model Parameters

In some embodiments, one challenge with GMM fitting and EM in particular is that it may be difficult to ensure that components initialized to a particular cluster of data remain around that data. Components may develop extremely large variances in order to better describe junk data at the tails of real clusters, which can also cause the components of the GMM to drift towards the geometric center of the data. To ameliorate this behavior, priors may be incorporated on the model parameters. These priors may bias the fitted results to some target value, which reduces the predictive power of the model but can help ensure that the clusters of interest remain well-described. Priors may also be used to generate better initial guesses for a subsequent prior-free optimization.

In some embodiments, Gaussian priors may be applied to the means and the variances of each cluster. The prior on the mean may be:

$$p ( {μ}_{j} ) = \frac{1}{2 π \sqrt{\vert {Λ}_{μ} \vert}} exp [ - \frac{1}{2} {( {μ}_{j} - {μ}_{j}^{db} )}^{T} {Λ}_{μ}^{- 1} ( {μ}_{j} - {μ}_{j}^{db} ) ]$$

where μⱼ^{db} is the expected position of the dye cluster center according to a reagents database and Λ_{μ} is the width of the prior on the mean. The prior on the variance may be:

$$p ( {\sum }_{j} ) = \frac{1}{2 π \sqrt{\vert {Λ}_{\sum } \vert}} exp [ - \frac{1}{2} Tr [ {\sum }_{j}^{T} {Λ}_{\sum }^{- 1} {\sum }_{j} ] ]$$

where Λ_{Σ} is the width of the prior on the variance. Unlike the means, there may be no established expected values for the variance. Instead, the prior may have the effect of penalizing large variances, as one form of pathological behavior during GMM fitting is that the variance explodes to large values.

### Combining Per-Pulse Uncertainties and Priors

In some embodiments, combining the priors with the per-pulse uncertainties yields a final expression for the posterior of the GMM:

$$p ( \{ {w}_{j} , {μ}_{j} , {\sum }_{j} \} \vert \{ {x}_{i} , {Δ}_{i} \} ) = {\prod }_{i = 1}^{N} {\sum }_{j = 1}^{K} \frac{{w}_{j}}{2 π \sqrt{\vert {\sum }_{j} + {Δ}_{i} \vert}} exp [ - \frac{1}{2} {( {x}_{i} - {μ}_{j} )}^{T} {( {\sum }_{j} + {Δ}_{i} )}^{- 1} ( {x}_{i} - {μ}_{j} ) ] \times {\prod }_{j = 1}^{K} \frac{1}{2 π \sqrt{\vert {Λ}_{μ} \vert}} exp [ - \frac{1}{2} {( {μ}_{j} - {μ}_{j}^{db} )}^{T} {Λ}_{μ}^{- 1} ( {μ}_{j} - {μ}_{j}^{db} ) ] \times {\prod }_{j = 1}^{K} \frac{1}{2 π \sqrt{\vert {Λ}_{\sum } \vert}} exp [ - \frac{1}{2} Tr [ {\sum }_{j}^{T} {Λ}_{\sum }^{- 1} {\sum }_{j} ] ]$$

The auxiliary function that is optimized in the M-step becomes:

$$A ( \{ {μ}_{j} , {\sum }_{j} \} \vert \{ {x}_{i} , {Δ}_{i} \} ) = {\sum }_{i = 1}^{N} {\sum }_{j = 1}^{K} {γ}_{ij} [ - log ( 2 π ) - \frac{1}{2} log \vert {\sum }_{j} + {Δ}_{i} \vert - \frac{1}{2} {( {x}_{i} - {μ}_{j} )}^{T} {( {\sum }_{j} + {Δ}_{i} )}^{- 1} ( {x}_{i} - {μ}_{j} ) ] + {\sum }_{j = 1}^{K} [ - log ( 2 π ) - \frac{1}{2} log \vert {Λ}_{μ} \vert - \frac{1}{2} {( {μ}_{j} - {μ}_{j}^{db} )}^{T} {Λ}_{μ}^{- 1} ( {μ}_{j} - {μ}_{j}^{db} ) ] + {\sum }_{j = 1}^{K} [ - log ( 2 π ) - \frac{1}{2} log \vert {Λ}_{\sum } 1 - \frac{1}{2} Tr [ {\sum }_{j}^{T} {Λ}_{\sum }^{- 1} {\sum }_{j} ] ]$$

The priors on the component means and variances manifest as L2 regularization penalties in the final auxiliary function. To determine the updated mean, the derivative of A with respect to μⱼ may be calculated, set equal to zero, and solved for μⱼ:

$${μ}_{j} = {[ {\sum }_{i = 1}^{N} {γ}_{ij} {( {\sum }_{j} + {Δ}_{i} )}^{- 1} + {∧}_{μ}^{- 1} ]}^{- 1} [ {\sum }_{i = 1}^{N} {γ}_{ij} {( {\sum }_{j} + {Δ}_{i} )}^{- 1} {x}_{i} + {∧}_{μ}^{- 1} {μ}_{j}^{db} ]$$

In a standard GMM, each observation is weighted by its responsibility γᵢⱼ, whereas in this approach, each pulse is additionally weighted by the inverse of the per-pulse variance. The second derivative of A with respect to μⱼ yields:

$$\frac{{\partial }^{2} A}{\partial {μ}_{j}^{2}} = {\sum }_{i = 1}^{N} {γ}_{ij} {( {\sum }_{j} + {Δ}_{i} )}^{- 1} - {∧}_{μ}^{- 1}$$

Since Σⱼ, Δᵢ, and Λ_{μ} are all positive definite, the second derivative is negative definite, so the M-step derived above is guaranteed to yield the global maximum of the auxiliary function with respect to μⱼ.

In some embodiments, the derivative of A with respect to Σⱼ may be more complicated. Assuming all matrices are diagonal (i.e., the off-diagonal elements are all 0), which may be valid when no correlation is observed between the bin ratio noise and the log (intensity) noise in the underlying pulse data, the derivative becomes:

$$\frac{\partial A}{\partial {\sum }_{j}} = {\sum }_{i = 1}^{N} [ - \frac{1}{2} {( {\sum }_{j} + {Δ}_{i} )}^{- 1} + \frac{1}{2} ( {x}_{i} - {μ}_{j} ) {( {x}_{i} - {μ}_{j} )}^{T} {( {\sum }_{j} + {Δ}_{i} )}^{- 2} ] - {∧}_{Σ}^{- 1} {\sum }_{j}$$

There may be no analytical solution to this expression, so it may be solved numerically. Since each component of Σⱼ can be solved independently of every other component under the diagonal matrix assumption, this can be done by performing Newton-Raphson on the diagonal components of the derivative. The second derivative of A with respect to Σⱼ is:

$$\frac{{\partial }^{2} A}{\partial {\sum }_{j}^{2}} = {\sum }_{i = 1}^{N} [ \frac{1}{2} {( {\sum }_{j} + {Δ}_{i} )}^{- 2} - ( {x}_{i} - {μ}_{j} ) {( {x}_{i} - {μ}_{j} )}^{T} {( {\sum }_{j} + {Δ}_{i} )}^{- 3} ] - {∧}_{Σ}^{- 1}$$

In principle, the second derivative of a scalar with respect to a matrix should be a tensor with 4 indices. However, since it is assumed that the components of the variance are separable, the tensor is being collapsed down to just the 2 indices of the variance matrix.

The second derivative is not negative definite, unlike the second derivative of A with respect to μⱼ. Thus, care may be taken to remain in the domain where the second derivative is negative. Since the second derivative is evaluated anyway to apply Newton-Raphson, it can be verified that the second derivative is negative, and if it is not, the Newton-Raphson step may be bypassed to instead shrink the value of Σⱼ. In practice, this approach may converge in around 6 iterations.

This yields a final EM algorithm for the modified GMM with per-pulse variances and priors on the component means and variances: (0) define hyperparameters Λ_{μ} and Λ_{Σ}; (1) guess initial parameters wⱼ, μⱼ, and Σⱼ; (2) E-step: calculate the responsibilities γᵢⱼ; (3) update wⱼ according to γᵢⱼ; (4) M-step: update μⱼ using the mean update formula; (5) M-step: iteratively maximize the auxiliary function with respect to Σⱼ by applying Newton-Raphson; and (6) return to step 2 and repeat until convergence.

Aside from the iterative M-step for Σⱼ, one other difference between this approach and standard GMM EM is that the overall global maximum of the auxiliary function is not necessarily found at every iteration. This is because the M-step for μⱼ depends on Σⱼ, and the M-step for Σⱼ depends on μⱼ. Thus, to find the global maximum, it would be needed to iterate the two M-steps until they converge. In contrast, the M-step for μⱼ in standard GMM EM does not depend on Σⱼ, so first updating μⱼ, then updating Σⱼ is guaranteed to reach the global maximum of the auxiliary function. This should not matter in practice, as convergence of the EM algorithm is predicated only on improving the log likelihood at every iteration. It is not required to find the global maximum of the auxiliary function at every iteration.

### Calculating Absolute Intensity

In some embodiments, while a reagents database may provide precise estimates for the average bin ratio for every dye cluster, it may only provide relative locations of the intensity centers. This is because a variety of factors may impact the exact intensities of each dye cluster. In practice, a sequencing device may attempt to tune laser intensity to target a particular intensity level, but this tuning may be rather coarse, resulting in run-to-run variation, particularly in recognition runs where not all dyes are observed.

In some embodiments, to determine the intensity offset of the run, a set of pulses (e.g., 200,000 pulses) with a minimum duration (e.g., 3 frames) may be collected from randomly chosen apertures and EM may be applied to optimize a GMM where the variances and relative center locations are fixed, and only the absolute intensity offset δ_{μ} is optimized. For this optimization, the E-step becomes:

$${γ}_{ij} = \frac{\begin{matrix}\frac{{w}_{j}}{2 π \sqrt{\vert {\sum }_{j}^{( 0 )} + {Δ}_{i} \vert}} \\ exp [ - \frac{1}{2} {( {x}_{i} - ( {μ}_{j}^{db} + {δ}_{μ} ) )}^{T} {( {\sum }_{j}^{( 0 )} + {Δ}_{i} )}^{- 1} ( {x}_{i} - ( {μ}_{j}^{db} + {δ}_{μ} ) ) ]\end{matrix}}{\begin{matrix}{\sum }_{{j}^{′} = 1}^{K} \frac{{w}_{j}^{′}}{2 π \sqrt{\vert {\sum }_{j ′}^{( 0 )} + {Δ}_{i} \vert}} \\ exp [ - \frac{1}{2} {( {x}_{i} - ( {μ}_{{j}^{′}}^{db} + {δ}_{μ} ) )}^{T} {( {\sum }_{{j}^{′}}^{( 0 )} + {Δ}_{i} )}^{- 1} ( {x}_{i} - ( {μ}_{j}^{db} + {δ}_{μ} ) ) ]\end{matrix}}$$

where μⱼ^{db} is the center of dye j according to the reagents database without offset and Σⱼ⁽⁰⁾ is the fixed guess variance for dye j. In some embodiments, a guess bin ratio variance of 0.0001 and log (intensity) variance of 0.001 may be used for all dyes.

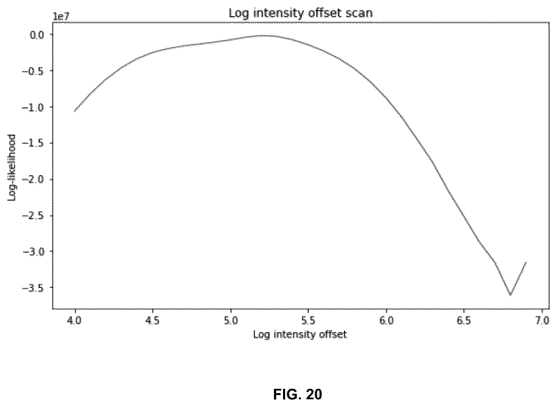

In some embodiments, to ensure that EM converges to the best possible offset, an initial scan of values between 4 and 7 log intensity units with a resolution of 0.1 log intensity units may be performed. From this scan, the log intensity offset that maximizes the log likelihood may be identified and used as an initial guess for the EM optimization. FIG. 20 illustrates a graph of log-likelihood as a function of log intensity offset for determining an intensity offset parameter, according to some embodiments of the technology described herein.

The auxiliary function for calculating the M-step is:

$$A ( {δ}_{μ} ) = {\sum }_{i = 1}^{N} {\sum }_{j = 1}^{K} {γ}_{ij} [ - log ( 2 π ) - \frac{1}{2} log \vert {\sum }_{j}^{( 0 )} + {Δ}_{i}$$

$$I - \frac{1}{2} {( {x}_{i} - ( {μ}_{j}^{db} + {δ}_{μ} ) )}^{T} {( {\sum }_{j}^{( 0 )} + {Δ}_{i} )}^{- 1} ( {x}_{i} - ( {μ}_{j}^{db} + {δ}_{μ} ) ) ]$$

This results in an M-step for the offset:

$$δμ = {[ {\sum }_{i = 1}^{N} {\sum }_{j = 1}^{K} {γ}_{ij} {( {\sum }_{j}^{( 0 )} + {Δ}_{i} )}^{- 1} ]}^{- 1} {\sum }_{i = 1}^{N} {\sum }_{j = 1}^{K} {γ}_{ij} {( {\sum }_{j}^{( 0 )} + {Δ}_{i} )}^{- 1} ( {x}_{i} - {μ}_{j}^{db} )$$

In practice, only an offset to the log intensity dimension may be applied, not the bin ratio dimension. This is equivalent to forcing the first component of δ_{μ} to always be zero regardless of the M-step. This algorithm may converge to a log-likelihood error of 10⁻⁶ in under 20 iterations. FIG. 21 illustrates a histogram of pulse bin ratios versus log-intensities with an initial guess dye caller overlaid, where the vertical position of the dye caller was determined by the intensity offset calculation, according to some embodiments of the technology described herein.

### Generating a Biased Sample of Pulses

In some embodiments, while all expected dye clusters may be roughly visible in a random sample of pulses, several of the clusters may be extremely dim, with one cluster in particular representing the majority of the pulses. While such a sample may be sufficient to identify an approximate intensity offset, fitting the centers and variances of the GMM components corresponding to the less prevalent dyes may be more difficult due to the presence of noise or junk pulses. Ideally, a GMM may be fit to a collection of pulses in which each dye is evenly represented.

In some embodiments, quantifying how many pulses are present for each dye would in principle require a dye caller, which poses a challenge when the purpose of the algorithm is to create a dye caller in the first place. However, for the purpose of evening out the distribution of dyes, a highly accurate dye caller may not be necessary, so the guess GMM used in the absolute intensity calculation step may suffice for the purpose of generating a biased sample of pulses.

In some embodiments, to generate the biased sample of pulses, pulses that would make the dyes less evenly represented may be filtered out. Rather than applying filtering to pulses within a given aperture (which runs the risk of outlier pulses from over-represented dye clusters being mislabeled as a less-prevalent dye), all pulses from a given aperture may be either accepted or rejected. The task then becomes finding a set of apertures that collectively give rise to a set of pulses that are evenly distributed among the dyes.

In some embodiments, the Metropolis-Hastings rejection sampling algorithm may be applied to the entropy of the dye counts. For a collection of pulses that have been classified as one of K dyes, the entropy may be defined as:

$$S = - {\sum }_{j = 1}^{K} \frac{{N}_{j}}{N} log ( \frac{{N}_{j}}{N} )$$

where S is the entropy, Nj is the number of pulses assigned to dye j, and N is the total number of pulses. If all dyes have the same number of pulses, the entropy will be maximized. If a new aperture would result in the entropy of the pulse collection increasing, then that aperture is accepted unconditionally. If the entropy would decrease, then the aperture is accepted with probability:

$${p}_{accept} = exp [ \frac{{S}^{+} - S}{NT} ]$$

where S⁺ is the new (lower) entropy if the pulses were to be accepted and T is the temperature, a hyperparameter controlling how aggressively the algorithm rejects apertures that lower the entropy. In addition to the temperature, the change in entropy may also be divided by the number of pulses in the sample to reduce sampling bias towards small samples. In some embodiments, a temperature of 10⁻⁸ may be used by default.

In some embodiments, the biased sampling algorithm may work as follows: (1) select an aperture at random, read in its pulses, and apply the per-aperture intensity correction factor, then group the pulses in the aperture based on pre-determined ROI calls; (2) using the guess dye caller, calculate the relative probability that each pulse was sampled from each dye component, then sum the probabilities across all pulses to get an approximate pulse count for each dye; (3) calculate the dye count entropy S with and without this aperture's pulses; (4) if the entropy would go up, accept the aperture's pulses unconditionally, and if the entropy would go down, accept with the calculated probability p_{accept}; and (5) return to step 1 and repeat until the target number of pulses have been collected.

FIGS. 22A-22C illustrate histograms comparing un-biased and biased selection of pulses for dye caller training, according to some embodiments of the technology described herein.

FIG. 22A is a histogram of pulse bin ratios vs. log of intensities. On the left, there is a histogram of un-biased random selection of 200,000 pulses. The histogram shows that the biased sampling algorithm may generate a more even representation of dyes than a purely random selection of pulses. On the right, there is a biased selection of 200,000 using the algorithm described in this section.

FIG. 22B is a histogram of pulse bin ratios vs. log of intensities for a recognition run with only a single observed dye. This algorithm may also work for recognition runs in which not all dyes are expected to be present, including extreme cases where recognition runs may only have a single dye present.

FIG. 22C is a histogram of pulse bin ratios vs. log of intensities for a different recognition run. The biased sample shows two faint clusters at high bin ratio, which likely correspond to dyes binding at a very low rate (e.g., due to promiscuous binding, dye sticking, or low concentrations of on-target peptide states due to unintentional peptide digestion, for example).

### Fitting the Classification Model

In some embodiments, once a collection of pulses with a more even representation of dyes has been collected, the classification model 106A may be trained on the data using the EM procedure outlined above. For an initial stage of optimization, the number of components may be set to the number of dyes plus one, with the extra component being used to fit the junk pulses between the clusters. The initial means for the components representing dyes may be set to the reagents database values, and the junk component center may be initialized to the mean of the database values. The variances of the clusters representing dyes may be initialized to zero (i.e., only the per-pulse variances are used), while the junk component variance may be set to 0.02 for the bin ratio and 0.2 for the log (intensity). FIG. 23A illustrates an initial guess of the dye caller prior to optimization, with the junk component represented as gray ellipses, according to some embodiments of the technology described herein.

In some embodiments, in the initial phase of optimization, Gaussian priors may be placed on the means and variances of the components corresponding to dyes (but not the junk component). The prior on the mean may be centered at the reagents database values, while the prior on the variance may be centered at a variance of 0. In practice, priors may be parameterized using their inverse variance, Λ_{μ}⁻¹ and ΛΣ⁻¹, and only the diagonal components may be non-zero. In order to ensure that the effect of the prior is not overwhelmed by the contribution of each pulse to the likelihood, the inverse prior variance may be scaled by the number of pulses. In some embodiments, the inverse prior variance on the mean bin ratio may be set to 1 times the number of pulses, and the inverse prior variance on the mean log (intensity) may be set to 0.5 times the number of pulses. The inverse prior variance on the bin ratio variance may be set to a number times the number of pulses (e.g., 1000 times the number of pulses), and the inverse prior variance on the log (intensity) variance may be set to a number times the number of pulses (e.g., 500 times the number of pulses). FIG. 23B illustrates an intermediate dye caller after initial optimization with priors, according to some embodiments of the technology described herein.

In some embodiments, once the initial classification model has been optimized, the fitted weights corresponding to every real dye component (excluding the junk component) may be inspected to determine which, if any, dyes are missing. If a component weight is found to be less than a threshold percentage (e.g., 5%) of the highest weight dye component, it may be considered to be missing and thus excluded from the subsequent fitting stage.

In some embodiments, with the remaining dyes, a final optimization may be performed in which the priors are disabled. This may be implemented in practice by setting the bin ratio and log (intensity) components of the mean and variance priors inverse variances to 0. FIG. 23C illustrates a final fitted dye caller, with the junk component omitted from the plot, according to some embodiments of the technology described herein.

Following the final optimization, any dye components that were found to be missing in the initial optimization may be re-added to the GMM. The means of these missing dyes may be taken to be the reagents database expected positions, and the variance may be taken to be the average variance of the dyes that were found. Finally, the junk component may be removed, and the classification model 106A may be constructed and returned.

### Applying the Classification Model

In some embodiments, while it is possible to use the classification model 106A to assign dyes to individual pulses, this may be problematic as dye clusters tend to overlap significantly. A sizable fraction of the pulse density originating from one cluster may be closer to another cluster's center, which would result in the appearance of a mixed ROI dye composition at best, or a misclassified ROI at worst. Rather, the classification model 106A may be used to assess the pulses within an ROI in aggregate to arrive at a most probable dye (and thus recognizer) assignment for each ROI.

In some embodiments, one way of accomplishing this would be to calculate which dye component results in the maximum likelihood given all of the pulses in an ROI. However, this tends to dramatically over-weight the importance of outlier pulses, resulting in the highest variance component always being chosen as the most likely assignment whenever outliers are present. Instead, dyes may be assigned to ROIs by performing EM on just the weights of the GMM using just the pulses within a particular ROI. This is conceptually similar to the first approach mentioned, but relaxes the assumption that only a single dye is present. This approach allows the junk component to represent outlier pulses, while the remaining inliers are associated instead with the actual dye clusters.

In some embodiments, the converged weights may reflect the apparent relative abundance of dyes within the ROI. In addition to handling outliers, this approach may also account for the overlap of dye clusters. The composition calculated by this approach may be a vector with one element per GMM component (including junk) that add to one. From this vector, a dye composition may be extracted by discarding the junk weight and re-normalizing so that the remaining weights add to one. The ROI may then be associated with the dye that has the highest composition, and that dye's composition may be added to the ROI calls as a dye composition value. The weight of the junk component may also be added to the ROI calls as an indeterminate fraction value. Both dye composition and indeterminate fraction may be used to filter ROIs. A low dye composition may indicate that the ROI cannot be unambiguously identified with a particular dye, while a high indeterminate fraction may indicate that many of the pulses within the ROI are outliers.

### Example Techniques for Scoring Alignments

In some embodiments, the alignment module 108 may be configured to use a quadratic alignment scoring model for determining alignment scores. Some embodiments may be configured to use the quadratic alignment scoring model for determining alignment scores instead of other scoring techniques described herein (e.g., as part of recognizer matching 402 and/or alignment selection 406 described herein with reference to FIGS. 4A-4B). The alignment score may have the following expression:

$$aln\_score = {\sum }_{i} {w}_{i} ( {s}_{binder match}^{( i )} + {s}_{PD match}^{( i )} + {s}_{gap}^{( i )} ) + {\sum }_{j} {s}_{del}^{( j )}$$

where the index i runs over all recognition segments in the read and the index j runs over all skipped residues in the reference in the alignment trajectory. The factor wᵢ is the weight of the i-th recognition segment, which may be equal to 1 over the number of consecutive recognition segments in the read adjacent to state i that are assigned the same recognizer as i. This may have the effect of down-weighting the alignment scores of reads with potentially over-split recognition segments. The values s_{binder match}, s_{PD match}, s_{gap}, and s_{del} are the binder match, PD match, gap, and deletion scores respectively. These terms may be dependent on the alignment trajectory. For a given read-reference pair, a dynamic programming algorithm may be used to find the alignment trajectory that maximizes the alignment score.

In some embodiments, the alignment module 108 may require that each recognition segment in the read be aligned to a state in the reference whose predicted recognizer corresponds to the recognizer assigned to that recognition segment. For this reason, the binder match score may be a constant value, such as 1.

In some embodiments, the PD match, gap, and deletion scores may all be variations on a truncated quadratic scoring function. The general expression for this scoring function may be:

$$s = - w \times min ( \frac{{x}^{2}}{{σ}^{2}} , 1 )$$

where w is the weight of the scoring component (i.e., the PD match score weight, gap score weight, or deletion score weight), x is some measure of deviation between the observed and predicted property, and σ is a scaling coefficient for the function. The parameter w may have the effect of scaling the scoring function vertically, while σ may have the effect of scaling the function horizontally.

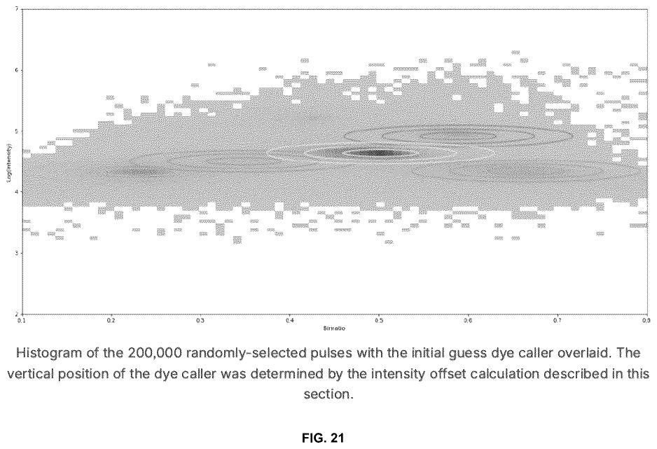

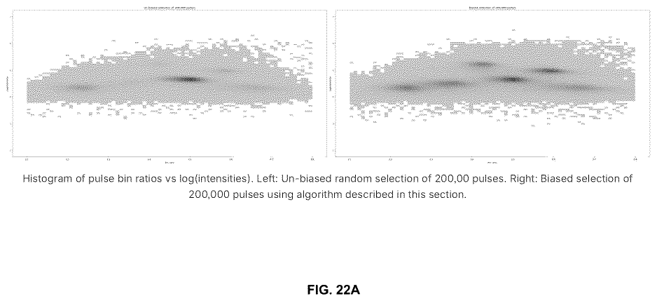

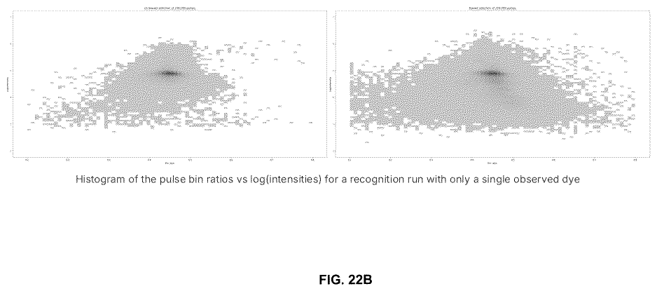

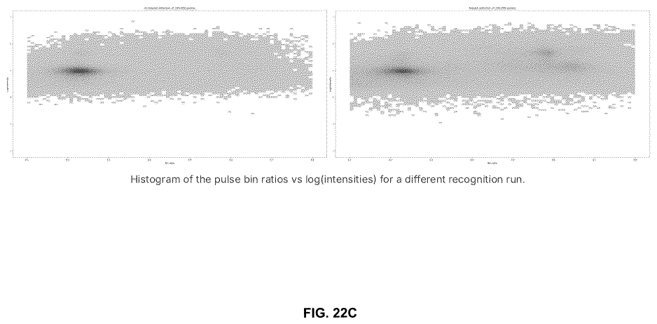

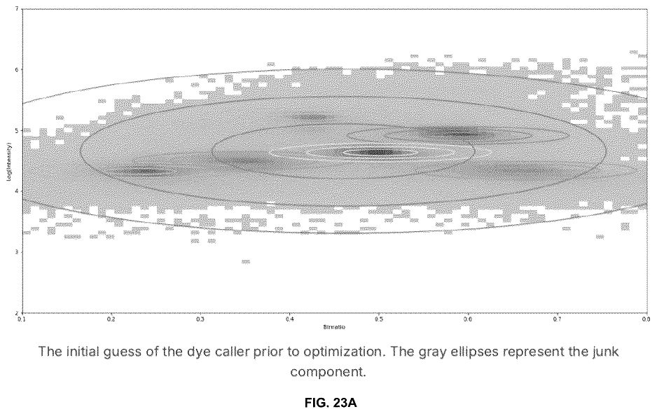

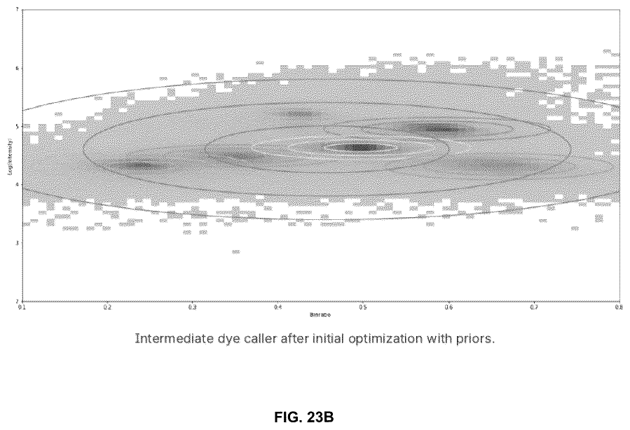

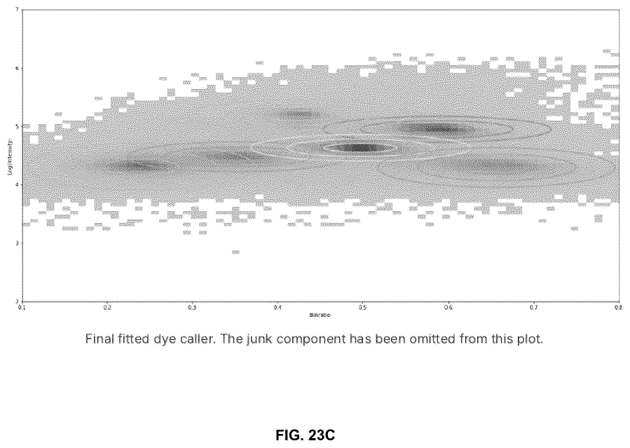

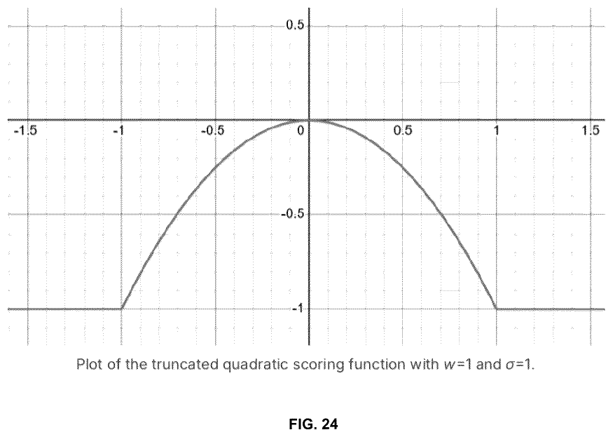

FIG. 24 illustrates a graph of a truncated quadratic scoring function used for alignment scoring, according to some embodiments of the technology described herein. The graph shows the scoring function with weight parameter w equal to 1 and scaling parameter σ equal to 1. The horizontal axis represents a variable x ranging from approximately −1.5 to 1.5, and the vertical axis represents the score value ranging from approximately −1 to 0.5. The curve has a parabolic shape in a central region between approximately x equals −1 and x equals 1, reaching a maximum value of 0 at x equals 0. The curve decreases quadratically from the maximum as x deviates from 0 in either direction, reaching a minimum value of approximately −1 at x equals −1 and x equals 1. Beyond these points, the curve is truncated and remains constant at approximately −1, forming horizontal line segments that extend to the edges of the graph.

In some embodiments, this functional form may have the following properties: the full functional space of the score can be controlled by 2 parameters that are minimally coupled; small deviations from expectation may be penalized less than large deviations; extremely large deviations may not incur arbitrarily large penalties, so a single bad measurement or bad prediction may not spoil an entire read; unlike a Gaussian function, the penalty may not slowly or asymptotically approach its lower bound but rather hard-clips to that bound; and the function may be negative semi-definite, thus being strictly a penalty for deviation from expectation. The function is computationally simple to evaluate.

In some embodiments, for the PD match score implementation of the quadratic function, x may be the difference between the logs of the predicted and observed pulse durations:

$${x}_{PD match} = ln ( {pd}_{obs} ) - ln ( {pd}_{pred} )$$

The σ of the PD match score may be a parameter of the model. In some embodiments, the σ of the PD match score may be optimized to a value of 1.8 (unitless). Similarly, the PD match score weight may be a parameter of the model that may be optimized to a value of 1.4 (score units).

In some embodiments, for the deletion score, x may be the predicted PD/IPD ratio for the state:

$${x}_{del} = \frac{{pd}_{pred}}{{ipd}_{pred}}$$

States with high predicted recognizer affinity may be more likely to be observed, and thus states with higher predicted PD/IPD ratios may incur a larger deletion penalty than states with low PD/IPD ratios. In some embodiments, both σ and w for the deletion score may be parameters of the model which may be optimized to 0.1 (unitless) and 0.4 (score units), respectively.

In some embodiments, for the gap score, x may be the difference between the observed gap duration (i.e., the amount of time that elapsed between the end of the previous recognition segment and the beginning of the current recognition segment) and the predicted gap duration, which may be the sum of the predicted recognition segment durations for each skipped residue in the reference:

$${x}_{gap} = {Δ}_{obs} - {\sum }_{j}^{skipped} {Δ}_{pred}^{( j )}$$

where Δ_{obs} is the observed gap duration and Δ_{pred}⁽ⁱ⁾ is the predicted mean recognition segment duration for reference state j. In the event that no residues were skipped in the reference, x_{gap}=Δ_{obs}.

In some embodiments, the parameter σ for the gap score may take a value that is dependent on whether any residues were skipped in the alignment trajectory. If no residues were skipped, then an optimized value for σ_{gap} of 5.4 (minutes) may be used. If at least one residue was skipped, then σ_{gap} may be calculated by the following expression:

$${σ}_{gap}^{2} = {a}_{gap} {\sum }_{j}^{skipped} {( {Δ}_{pred}^{( j )} )}^{2}$$

where α_{gap} is a parameter of the model that may be optimized to 1.8 (unitless). This expression may be equal to the variance of the hypoexponential (or generalized Erlang) distribution, which represents the distribution on wait times for k sequential events to occur, each of which has its own unique exponential rate. This may be a reasonable model for the distribution of gap times one would expect to observe between two visible residues in a reference that are separated by one or more invisible residues.

In some embodiments, the parameters of the quadratic alignment scoring model may be optimized using a Bayesian optimizer such as Optuna. The optimization may attempt to maximize alignment accuracy for a given target number of alignments. Each trial of the optimization may perform the following steps: a set of parameters (w_{PD match}, w_{gap}, w_{del}, σ_{PD match}, σ_{gap}, σ_{del}, α_{gap}) may be proposed by the optimizer; each of a plurality of single-protein runs may be aligned against their respective on-target reference, as well as an off-target reference; using the on-target alignments for each run, a value for the minimum alignment score which yields an average number of alignments equal to some target value may be determined; and using the on- and off-target alignments, and the minimum alignment score calculated by the previous step, the average alignment accuracy may be evaluated and returned as the trial function value.

The optimizer may then attempt to maximize the average alignment accuracy. This is a more efficient method for optimizing the aligner parameters, because it targets the specific value to maximize (the accuracy) given a constraint to meet in practice (matching the average number of alignments), and it does so without requiring multi-objective optimization.

In some embodiments, the optimized parameters may include: w_{PD match}=1.4; w_{gap}=0.69; w_{del}=0.40; σ_{PD match}=1.8; σ_{gap}=5.4 minutes; σ_{del}=0.10; α_{gap}=1.8; and a minimum alignment score of 3.14.

In some embodiments, the quadratic alignment scoring model may be evaluated against sequencing runs using a sequencing kit. The evaluation may include single protein runs, multi-protein mix runs, control peptide runs, and barcode peptide runs.

In some embodiments, for single protein runs, the quadratic alignment scoring model may result in approximately 9% more alignments compared to a previous alignment model. When excluding runs that appear to have failed based on extremely low alignment counts or extremely low accuracy, the quadratic alignment scoring model may achieve an alignment accuracy of approximately 98.91% compared to approximately 98.61% for another alignment model described above. Table 1 below summarizes example results for single protein runs excluding seemingly failed runs:

TABLE 1

| | Quadratic | Other |
|---|---|---|
| Metric | Alignment Model | Alignment Model |
| High-Quality Reads | 108974.5 | 92183.66 |
| Alignments | 27654.94 | 25329.11 |
| Peptides Identified | 8 | 7.03 |
| On-Target Alignment Accuracy | 98.91 | 98.61 |
| FDR | 0.0004 | 0.0019 |
| ROC-AUC | 0.87 | 0.85 |
| Average Precision | 1 | 1 |
| True protein rank | 1 | 1 |
| Number of proteins inferred | 52.77 | 19.66 |

In some embodiments, for multi-protein mix runs (e.g., 10 protein mix runs), the quadratic alignment scoring model may result in approximately 9% more alignments while increasing alignment accuracy from approximately 98.72% to approximately 99.15%. Table 2 below summarizes example results for 10 protein mix runs:

TABLE 2

| | Quadratic | Other |
|---|---|---|
| Metric | Alignment Model | Alignment Model |
| High-Quality Reads | 201534.17 | 170005.33 |
| Alignments | 52182.67 | 48086.89 |
| Peptides Identified | 56.94 | 45.17 |
| On-Target Alignment Accuracy | 99.15 | 98.72 |
| FDR | 0.01 | 0.02 |
| ROC-AUC | 0.81 | 0.77 |
| Average Precision | 0.82 | 0.72 |
| First true protein rank | 1.00 | 1.00 |
| Last true protein rank | 11774.11 | 11098.50 |
| Number of proteins inferred | 94.11 | 34.56 |

In some embodiments, for control peptide runs, the quadratic alignment scoring model may result in a reduction in alignment count (e.g., approximately 20% fewer alignments) while achieving a slight increase in alignment accuracy. Table 3 below summarizes example results for control peptide runs:

TABLE 3

| | Quadratic | Previous |
|---|---|---|
| Metric | Alignment Model | Alignment Model |
| High-Quality Reads | 231791.99 | 209761.28 |
| Alignments | 53274.67 | 66613.67 |
| Peptides Identified | 6.00 | 6.00 |
| On-Target Alignment Accuracy | 98.65 | 98.53 |
| FDR | 0.00 | 0.00 |
| ROC-AUC | 0.90 | 0.91 |

In some embodiments, for barcode peptide runs, the quadratic alignment scoring model may be combined with an increased maximum inter-pulse duration cutoff (e.g., from 20 seconds to 50 seconds) to achieve a substantial increase in alignments. Table 4 below summarizes example results for barcode peptide runs:

TABLE 4

| | Quadratic | Previous |
|---|---|---|
| Metric | Alignment Model | Alignment Model |
| High-Quality Reads | 226394.00 | 160596.39 |
| Alignments | 71541.94 | 34668.78 |
| Peptides Identified | 24.00 | 23.89 |
| FDR | 0.00 | 0.01 |
| ROC-AUC | 0.88 | 0.80 |

In some embodiments, the increase in alignments for barcode peptides may be attributed to the increased maximum inter-pulse duration cutoff rather than the quadratic alignment scoring model itself. Barcode peptides may exhibit states with inter-pulse durations above a previous maximum cutoff threshold, and increasing the maximum inter-pulse duration cutoff may allow these states to be included in alignments without negatively impacting performance for single protein, multi-protein mix, or control peptide runs.

In some embodiments, the quadratic alignment scoring model may achieve an overall increase in alignment count, alignment accuracy, and inference precision for sequencing kit runs including single protein and multi-protein mix runs relative to other scoring models described herein. The reduction in alignment count for control peptides may be offset by the improvement in performance for runs of real protein samples. The quadratic alignment scoring model may provide improved performance characteristics for protein sequencing applications while maintaining computational efficiency through the use of the truncated quadratic scoring function.

### LIST OF EXAMPLE EMBODIMENTS

1. In some embodiments, the techniques described herein relate to a method for detecting amino acid variants in peptides using data obtained by a sequencing device, the method including: using at least one computer hardware processor to perform: obtaining sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assigning, to recognition segments in the plurality of reads, fluorescently tagged NAA recognizers determined to be binding in the recognition segments at least in part by using: (1) the light pulse durations, and (3) the inter-pulse durations; and detecting one or more amino acid variants in the at least one peptide using the plurality of reads and an assignment of the fluorescently tagged NAA recognizers to the recognition segments of the plurality of reads.
2. In some embodiments, the techniques described herein relate to a method, wherein assigning, to the recognition segments in the plurality of reads, the fluorescently tagged NAA recognizers determined to be binding in the recognition segments includes, for each of the recognition segments: obtaining fluorescence data for the recognition segment, the fluorescence data indicating detected fluorescence intensity and fluorescence decay of at least one fluorescent dye of at least one fluorescently tagged NAA recognizer binding in the recognition segment; and identifying, using the fluorescence data for the recognition segment, the at least one fluorescently tagged NAA recognizer from among a set of candidate fluorescently tagged NAA recognizers for assignment to the recognition segment.
3. In some embodiments, the techniques described herein relate to a method, wherein detecting the one or more amino acid variants in the at least one peptide using the plurality of reads and the assignment of the fluorescently tagged NAA recognizers to the recognition segments of the plurality of reads includes: aligning at least one of the plurality of reads to each of one or more reference peptide sequences to obtain one or more peptide alignments at least in part by using the assignment of the fluorescently tagged NAA recognizers to the recognition segments of the plurality of reads; and performing the detecting of the one or more amino acid variants using the one or more peptide alignments.
4. In some embodiments, the techniques described herein relate to a method, wherein aligning the at least one read to each of the one or more reference peptide sequences to obtain the one or more peptide alignments includes: filtering out a subset of recognition segments from the at least one read to obtain a filtered at least one read.
5. In some embodiments, the techniques described herein relate to a method, wherein filtering out the subset of recognition segments from the at least one read includes: determining mean inter-pulse durations in recognition segments of the at least one read; identifying, as the subset of recognition segments, recognition segments in which a mean inter-pulse duration is greater than a threshold inter-pulse duration; and removing the subset of recognition segments from the at least one read to obtain the filtered at least one read.
6. In some embodiments, the techniques described herein relate to a method, wherein filtering out the subset of recognition segments from the at least one read includes: determining dye purities for fluorescently tagged NAA recognizers assigned to recognition segments of the at least one read; identifying, as the subset of recognition segments, recognition segments for which a dye purity is less than a threshold dye purity; and removing the subset of recognition segments from the at least one read to obtain the filtered at least one read.
7. In some embodiments, the techniques described herein relate to a method, wherein filtering out the subset of recognition segments from the at least one read includes: determining dye composition distances for fluorescently tagged NAA recognizers assigned to recognition segments of the at least one read; identifying, as the subset of recognition segments, recognition segments for which a dye composition distance is above a threshold dye composition distance; and removing the subset of recognition segments from the at least one read to obtain the filtered at least one read.
8. In some embodiments, the techniques described herein relate to a method, wherein aligning the at least one read to each of one or more reference peptide sequences includes: accessing, for amino acid residues in the reference peptide sequence, expected fluorescently tagged NAA recognizers; and assigning recognition segments in the at least one read to amino acid residues in the reference peptide sequence at least in part by matching fluorescently tagged NAA recognizers assigned to the recognition segments to expected fluorescently tagged NAA recognizers of the amino acid residues in the reference peptide sequence.
9. In some embodiments, the techniques described herein relate to a method, wherein aligning the at least one read to each of the one or more reference peptide sequences includes: generating multiple candidate alignments with the reference peptide sequence; and determining alignment scores for the candidate alignments; and selecting, using the alignment scores determined for the candidate alignments, one of the candidate alignments as an alignment of the at least one peptide with the reference peptide sequence.
10. In some embodiments, the techniques described herein relate to a method, wherein determining the alignment scores the candidate alignments includes, for each of the candidate alignments: obtaining expected light pulse durations for amino acid residues in the reference peptide sequence; comparing light pulse durations of recognition segments in the at least one read to expected light pulse durations of respective amino acid residues in the reference peptide sequence with which the recognition segments are aligned; and determining an alignment score for the candidate alignment using a result of comparing the light pulse durations of the recognition segments to the expected light pulse durations of the respective amino acid residues in the reference peptide sequence.
11. In some embodiments, the techniques described herein relate to a method, wherein obtaining the expected light pulse durations for the amino acid residues in the reference peptide sequence includes: accessing a reference dataset storing pulse durations for amino acid motifs; and determining, using the pulse durations for the amino acid motifs, the expected light pulse durations for the amino acid residues in the reference peptide sequence.
12. In some embodiments, the techniques described herein relate to a method, wherein determining, using the pulse durations for the amino acid motifs, the expected light pulse durations for the amino acid residues in the reference peptide sequence includes, for each of at least some of the amino acid residues in the reference peptide sequence: identifying a subsequence of the reference peptide consisting of the amino acid residue and one or more preceding amino acid residues; identifying, in the reference dataset, one of the amino acid motifs using the subsequence; and determining, as an expected pulse duration for the amino acid residue, a pulse duration stored for the identified amino acid motif in the reference dataset.
13. In some embodiments, the techniques described herein relate to a method, further including determining at least some of the pulse durations stored in the reference dataset for at least some of the amino acid motifs using a trained machine learning model to predict pulse durations.
14. In some embodiments, the techniques described herein relate to a method, wherein determining the at least some pulse durations for the at least some amino acid motifs using the trained machine learning model to predict the pulse durations includes: generating sets of features for the at least some amino acid motifs; and providing the sets of features as input to the machine learning model to obtain output indicating the at least some pulse durations for the at least some amino acid motifs.
15. In some embodiments, the techniques described herein relate to a method, wherein generating the sets of features for the at least some amino acid motifs includes, for each of the at least some amino acid motifs: generating a one-hot encoding of amino acids in the amino acid motif; generating a sinusoidal positional encoding of amino acid positions in the amino acid motif; and generating a set of features for the amino acid motif at least in part by combining the one-hot encoding and the sinusoidal positional encoding.
16. In some embodiments, the techniques described herein relate to a method, wherein the trained machine learning model includes a neural network, the neural network including: a plurality of fully connected layers including: a first layer configured to receive a combination of an input one-hot encoding with an input sinusoidal positional encoding generated for a particular amino acid motif; and an output layer configured to output a pulse duration prediction for the particular amino acid motif.
17. In some embodiments, the techniques described herein relate to a method, wherein comparing the light pulse durations of the recognition segments to the expected light pulse durations of the respective amino acid residues in the reference peptide sequence includes: determining differences between mean light pulse durations of the recognition segments and the expected light pulse durations of the respective amino acid residues in the reference peptide sequence; and determining a component of the alignment score using the differences between the mean light pulse durations of the recognition segments and the expected light pulse durations of the respective amino acid residues in the reference peptide sequence.
18. In some embodiments, the techniques described herein relate to a method, wherein determining the alignment scores for the candidate alignments includes, for each of the candidate alignments: identifying positions in the candidate alignment where amino acid residues of the reference peptide sequence have expected fluorescently tagged NAA recognizers but are not aligned with any recognition segment in the at least one read; and determining an alignment score for the candidate alignment based on the identified positions.
19. In some embodiments, the techniques described herein relate to a method, wherein determining the alignment score for the candidate alignment based on the identified positions includes: accessing expected pulse durations for the amino acid residues of the reference peptide sequence at the identified positions; determining a deletion penalty using the expected pulse durations of the amino acid residues of the reference peptide sequence; and determining the alignment score for the candidate alignment using the deletion penalty.
20. In some embodiments, the techniques described herein relate to a method, wherein determining the alignment scores for the candidate alignments includes, for each of the candidate alignments: determining a gap score for the candidate alignment based on spacing between recognition segments of the at least one read relative to the reference peptide sequence; and determining an alignment score for the candidate alignment using the gap score.
21. In some embodiments, the techniques described herein relate to a method, wherein determining the gap score for the candidate alignment based on the spacing between the recognition segments of the at least one read relative to the reference peptide sequence includes: identifying positions in the candidate alignment where: two adjacent recognition segments in the at least one read are aligned with a common amino acid residue in the reference peptide sequence; and an amount of time between the two adjacent recognition segments is greater than or equal to a threshold amount of time; and determining the gap score based on the identified positions.
22. In some embodiments, the techniques described herein relate to a method, wherein determining the gap score for the candidate alignment based on the spacing between the recognition segments of the at least one read relative to the reference peptide sequence includes: identifying positions in the candidate alignment where: two recognition segments in the at least one read align to adjacent amino acid residues in the reference peptide sequence; and an amount of time between the two recognition segments is less than or equal to a threshold amount of time; and determining the gap score based on the identified positions.
23. In some embodiments, the techniques described herein relate to a method, wherein determining the gap score for the candidate alignment based on the spacing between the recognition segments of the at least one read relative to the reference peptide sequence includes: identifying positions in the candidate alignment where: an amino acid residue in the reference peptide sequence is not aligned with any recognition segment of the at least one read and is between two adjacent recognition segments of the at least one read; and an amount of time between the two adjacent recognition segments is in a particular time range; and determining the gap score based on the identified positions.
24. In some embodiments, the techniques described herein relate to a method, wherein determining the gap score for the candidate alignment based on the spacing between the recognition segments of the at least one read relative to the reference peptide sequence includes: identifying portions of the candidate alignment in which: two or more contiguous amino acid residues of the reference peptide sequence are: (1) not aligned with any recognition segments of the at least one read, and (2) between two adjacent recognition segments of the at least one read; and an amount of time between the two adjacent recognition segments is greater than a threshold amount of time; and determining the gap score based on the identified portions of the candidate alignment.
25. In some embodiments, the techniques described herein relate to a method, wherein the method further includes using the at least one computer hardware processor to perform: determining, for each of the plurality of reads, whether recognition segments of the read have been assigned a threshold number of fluorescently tagged NAA recognizers; and when it is determined that recognition segments of the at least one read have been assigned the threshold number of fluorescently tagged NAA recognizers, performing the aligning of the at least one read to the one or more reference peptide sequences.
26. In some embodiments, the techniques described herein relate to a method, further including: determining, for each of the plurality of reads, whether a length of the read is at least a threshold number of recognition segments after collapsing contiguous portions of the read that are assigned a common fluorescently tagged NAA recognizer; and when it is determined that a length of the at least one read is at least the threshold number of recognition segments, performing the aligning of the at least one read to the one or more reference peptide sequences.
27. In some embodiments, the techniques described herein relate to a method, wherein detecting the one or more amino acid variants in the at least one peptide using the plurality of reads and the assignment of the fluorescently tagged NAA recognizers to the recognition segments of the plurality of reads includes: determining, using the assignment of the fluorescently tagged NAA recognizers to the recognition segments of the plurality of reads, amino acid variant identities of the plurality of reads.
28. In some embodiments, the techniques described herein relate to a method, wherein determining, using the assignment of the fluorescently tagged NAA recognizers to the recognition segments of the plurality of reads, amino acid variant identities of the plurality of reads includes: determining the amino acid variant identities of the plurality of reads using a trained machine learning model.
29. In some embodiments, the techniques described herein relate to a method, wherein the trained machine learning model includes a classification model, and the method further includes training the classification model by: clustering the plurality of reads to obtain multiple classes each corresponding to a particular amino acid variant, wherein determining the amino acid variant identities of the plurality of reads using the trained machine learning model includes: classifying each of at least some of the plurality of reads into one of the classes to obtain an amino acid variant identity of the read.
30. In some embodiments, the techniques described herein relate to a method, wherein clustering the plurality of reads to obtain the multiple classes includes: clustering the plurality of reads using at least one of: dynamic time warping or k-means clustering.
31. In some embodiments, the techniques described herein relate to a system for identifying amino acid variants in peptides using data obtained by a sequencing device, the system including: the sequencing device, the sequencing device configured to obtain sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to: generate, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assign, to recognition segments in the plurality of reads, fluorescently tagged NAA recognizers determined to be binding in the recognition segments at least in part by using: (1) the light pulse durations, and (3) the inter-pulse durations; and detect one or more amino acid variants in the at least one peptide using the plurality of reads and an assignment of the fluorescently tagged NAA recognizers to the recognition segments of the plurality of reads.
32. In some embodiments, the techniques described herein relate to a non-transitory computer-readable storage medium storing instructions that, when executed by at least one computer hardware processor, cause the at least one computer hardware processor to perform a method for identifying amino acid variants in peptides using data obtained by a sequencing device, the method including: obtaining sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; assigning, to recognition segments in the plurality of reads, fluorescently tagged NAA recognizers determined to be binding in the recognition segments at least in part by using: (1) the light pulse durations, and (3) the inter-pulse durations; and detecting one or more amino acid variants in the at least one peptide using the plurality of reads and an assignment of the fluorescently tagged NAA recognizers to the recognition segments of the plurality of reads.
33. In some embodiments, the techniques described herein relate to a method for generating reads using data obtained by a sequencing device from detecting light emissions by fluorescently tagged N-terminal amino acid (NAA) recognizers during sequencing of at least one peptide, the method including: using at least one computer hardware processor to perform: obtaining sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.
34. In some embodiments, the techniques described herein relate to a method, wherein identifying, using the light pulse durations and the inter-pulse durations, the sequence of recognition segments in each read of the plurality of reads includes: determining mean inter-pulse durations of multiple light pulse windows; determining inter-pulse durations between final light pulses of the light pulse windows and respective subsequent light pulses; comparing mean inter-pulse durations of the light pulse windows to the inter-pulse durations between the final light pulses of the light pulse windows and the respective subsequent light pulses; and dividing the light pulses into proto-recognition segments based on a result of comparing the mean inter-pulse durations of the light pulse windows to the inter-pulse durations between the final light pulses of the light pulse windows and the respective subsequent light pulses; and dividing the proto-recognition segments to obtain the at least one sequence of recognition segments.
35. In some embodiments, the techniques described herein relate to a method, wherein dividing the proto-recognition segments to obtain the sequence of recognition segments includes: for each of at least some of the proto-recognition segments: comparing sequential pairs of light pulse windows in the proto-recognition segment; and dividing the proto-recognition segment into multiple recognition segments based on a result of comparing the sequential pairs of light pulse windows.
36. In some embodiments, the techniques described herein relate to a method, wherein: comparing the sequential pairs of light pulse windows includes, for each of the sequential pairs of light pulse windows: comparing a first measurement of at least one light pulse property in a first light pulse window in the pair of light pulse windows to a second measurement of the at least one light pulse property in a second light pulse window in the pair of light pulse windows; and applying at least one statistical test on the at least one light pulse property using a result of comparing the first measurement to the second measurement to obtain output indicating a probability that the first light pulse window and the second light pulse window correspond to a common binding interaction between one or more of the fluorescently tagged NAA recognizers and a particular NAA of the at least one peptide; and dividing the proto-recognition segment into multiple recognition segments based on the result of comparing the sequential pairs of light pulse windows includes: dividing the proto-recognition segment into the multiple recognition segments using outputs obtained from statistical tests applied on the at least one light pulse property for the sequential pairs of light pulse windows.
37. In some embodiments, the techniques described herein relate to a method, wherein the at least one light pulse property includes one or more of light pulse duration, inter-pulse duration, fluorescence intensity, and fluorescence decay.
38. In some embodiments, the techniques described herein relate to a method, wherein the at least one statistical test includes a Kolmogorov-Smirnov (KS) test.
39. In some embodiments, the techniques described herein relate to a method, further including assigning, to recognition segments in the plurality of reads, fluorescently tagged NAA recognizers determined to be binding in the recognition segments.
40. In some embodiments, the techniques described herein relate to a method, wherein assigning, to the recognition segments in the plurality of reads, the fluorescently tagged NAA recognizers determined to be binding in the recognition segments includes, for each of the recognition segments: obtaining fluorescence data for the recognition segment, the fluorescence data indicating detected fluorescence intensity and fluorescence decay of at least one fluorescent dye of at least one fluorescently tagged NAA recognizer binding in the recognition segment; and identifying, using the fluorescence data for the recognition segment, the at least one fluorescently tagged NAA recognizer from among a set of candidate fluorescently tagged NAA recognizers for assignment to the recognition segment.
41. In some embodiments, the techniques described herein relate to a system for generating reads using data obtained by a sequencing device from detecting light emissions by fluorescently tagged N-terminal amino acid (NAA) recognizers during sequencing of at least one peptide, the method including: a sequencing device, the sequencing device configured to obtain sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to perform: generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.
42. In some embodiments, the techniques described herein relate to a system, wherein identifying, using the light pulse durations and the inter-pulse durations, the sequence of recognition segments in each read of the plurality of reads includes: determine mean inter-pulse durations of multiple light pulse windows; determine inter-pulse durations between final light pulses of the light pulse windows and respective subsequent light pulses; compare mean inter-pulse durations of the light pulse windows to the inter-pulse durations between the final light pulses of the light pulse windows and the respective subsequent light pulses; and divide the light pulses into proto-recognition segments based on a result of comparing the mean inter-pulse durations of the light pulse windows to the inter-pulse durations between the final light pulses of the light pulse windows and the respective subsequent light pulses; and divide the proto-recognition segments to obtain the at least one sequence of recognition segments.
43. In some embodiments, the techniques described herein relate to a system, wherein dividing the proto-recognition segments to obtain the sequence of recognition segments includes: for each of at least some of the proto-recognition segments: compare sequential pairs of light pulse windows in the proto-recognition segment; and divide the proto-recognition segment into multiple recognition segments based on a result of comparing the sequential pairs of light pulse windows.
44. In some embodiments, the techniques described herein relate to a system, wherein: comparing the sequential pairs of light pulse windows includes, for each of the sequential pairs of light pulse windows: compare a first measurement of at least one light pulse property in a first light pulse window in the pair of light pulse windows to a second measurement of the at least one light pulse property in a second light pulse window in the pair of light pulse windows; and apply at least one statistical test on the at least one light pulse property using a result of comparing the first measurement to the second measurement to obtain output indicating a probability that the first light pulse window and the second light pulse window correspond to a common binding interaction between one or more of the fluorescently tagged NAA recognizers and a particular NAA of the at least one peptide; and dividing the proto-recognition segment into multiple recognition segments based on the result of comparing the sequential pairs of light pulse windows includes: divide the proto-recognition segment into the multiple recognition segments using outputs obtained from statistical tests applied on the at least one light pulse property for the sequential pairs of light pulse windows.
45. In some embodiments, the techniques described herein relate to a system, wherein the at least one light pulse property includes one or more of light pulse duration, inter-pulse duration, fluorescence intensity, and fluorescence decay.
46. In some embodiments, the techniques described herein relate to a system, wherein the at least one statistical test includes a Kolmogorov-Smirnov (KS) test.
47. In some embodiments, the techniques described herein relate to a system, further the instructions further cause the at least one computer hardware processor to: assign, to recognition segments in the plurality of reads, fluorescently tagged NAA recognizers determined to be binding in the recognition segments.
48. In some embodiments, the techniques described herein relate to a system, wherein assigning, to the recognition segments in the plurality of reads, the fluorescently tagged NAA recognizers determined to be binding in the recognition segments includes, for each of the recognition segments: obtain fluorescence data for the recognition segment, the fluorescence data indicating detected fluorescence intensity and fluorescence decay of at least one fluorescent dye of at least one fluorescently tagged NAA recognizer binding in the recognition segment; and identify, using the fluorescence data for the recognition segment, the at least one fluorescently tagged NAA recognizer from among a set of candidate fluorescently tagged NAA recognizers for assignment to the recognition segment.
49. In some embodiments, the techniques described herein relate to a non-transitory computer-readable storage medium storing instructions that, when executed by at least one computer hardware processor, cause the at least one computer hardware processor to perform a method for generating reads using data obtained by a sequencing device from detecting light emissions by fluorescently tagged N-terminal amino acid (NAA) recognizers during sequencing of at least one peptide, the method including: obtaining sequencing data generated from traces of light pulses output by the sequencing device from detecting the light emissions by the fluorescently tagged NAA recognizers in response to illumination during sequencing of the at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide, the generating including identifying a sequence of recognition segments in each read of the plurality of reads.
50. In some embodiments, the techniques described herein relate to a non-transitory computer-readable storage medium, wherein identifying, using the light pulse durations and the inter-pulse durations, the sequence of recognition segments in each read of the plurality of reads includes: determining mean inter-pulse durations of multiple light pulse windows; determining inter-pulse durations between final light pulses of the light pulse windows and respective subsequent light pulses; comparing mean inter-pulse durations of the light pulse windows to the inter-pulse durations between the final light pulses of the light pulse windows and the respective subsequent light pulses; and dividing the light pulses into proto-recognition segments based on a result of comparing the mean inter-pulse durations of the light pulse windows to the inter-pulse durations between the final light pulses of the light pulse windows and the respective subsequent light pulses; and dividing the proto-recognition segments to obtain the at least one sequence of recognition segments.
51. In some embodiments, the techniques described herein relate to a non-transitory computer-readable storage medium, wherein dividing the proto-recognition segments to obtain the sequence of recognition segments includes: for each of at least some of the proto-recognition segments: comparing sequential pairs of light pulse windows in the proto-recognition segment; and dividing the proto-recognition segment into multiple recognition segments based on a result of comparing the sequential pairs of light pulse windows.
52. In some embodiments, the techniques described herein relate to a non-transitory computer-readable storage medium, wherein: comparing the sequential pairs of light pulse windows includes, for each of the sequential pairs of light pulse windows: comparing a first measurement of at least one light pulse property in a first light pulse window in the pair of light pulse windows to a second measurement of the at least one light pulse property in a second light pulse window in the pair of light pulse windows; and applying at least one statistical test on the at least one light pulse property using a result of comparing the first measurement to the second measurement to obtain output indicating a probability that the first light pulse window and the second light pulse window correspond to a common binding interaction between one or more of the fluorescently tagged NAA recognizers and a particular NAA of the at least one peptide; and dividing the proto-recognition segment into multiple recognition segments based on the result of comparing the sequential pairs of light pulse windows includes: dividing the proto-recognition segment into the multiple recognition segments using outputs obtained from statistical tests applied on the at least one light pulse property for the sequential pairs of light pulse windows.
53. In some embodiments, the techniques described herein relate to a method for identifying amino acid residues in peptides using data obtained by a sequencing device from detecting light emissions by fluorescently tagged N-terminal amino acid (NAA) recognizers during sequencing of the peptides, the method including: using at least one computer hardware processor to perform: obtaining sequencing data generated from traces of light pulses output by the sequencing device from detecting light emissions by fluorescently tagged NAA recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assigning, to recognition segments in the plurality of reads, fluorescently tagged NAA recognizers determined to be binding in the recognition segments.
54. In some embodiments, the techniques described herein relate to a method, wherein assigning, to the recognition segments in the sequence of recognition segments, the fluorescently tagged NAA recognizers determined to be binding in the recognition segments includes, for each of the recognition segments: obtaining fluorescence data for the recognition segment, the fluorescence data indicating detected fluorescence intensity and fluorescence decay of at least one fluorescent dye of at least one fluorescently tagged NAA recognizer binding in the recognition segment; and identifying, using the fluorescence data for the recognition segment, the at least one fluorescently tagged NAA recognizer from among a set of candidate fluorescently tagged NAA recognizers for assignment to the recognition segment.
55. In some embodiments, the techniques described herein relate to a method, wherein identifying, using the fluorescence data for the recognition segment, the at least one fluorescently tagged NAA recognizer from among the set of candidate fluorescently tagged NAA recognizers for assignment to the recognition segment includes: processing, using a classification model trained to classify fluorescence data as corresponding to one of the set of candidate fluorescently tagged NAA recognizers, the fluorescence data for the recognition segment to determine a classification of the fluorescence data as corresponding to the at least one fluorescently tagged NAA recognizer.
56. In some embodiments, the techniques described herein relate to a method, wherein the classification model includes a Gaussian Mixture Model (GMM) classifier.
57. In some embodiments, the techniques described herein relate to a method, further including training the classification model, the training including: obtaining fluorescence data for the light pulses emitted by the fluorescently tagged NAA recognizers during the sequencing of the at least one peptide; and training the classification model using the fluorescence data.
58. In some embodiments, the techniques described herein relate to a method, wherein obtaining the fluorescence data for the light pulses includes, for each of multiple ones of the light pulses: log of fluorescence intensity detected after illumination that caused emission of the light pulse; and a ratio between: (1) a number of photons detected in a first time bin after the illumination, and (2) a number of photons detected in a second time bin after the illumination, wherein the second time bin is subsequent to the first time bin.
59. In some embodiments, the techniques described herein relate to a method, wherein processing, using the classification model, the fluorescence data to determine the classification of the fluorescence data as corresponding to the at least one fluorescently tagged NAA recognizer includes: determining a measure of similarity between the fluorescence data and each of multiple classes of the classification model, the multiple classes each corresponding to at least one candidate fluorescently tagged NAA recognizer of the candidate fluorescently tagged NAA recognizers; and selecting, as the classification, using similarity measurements determined for the multiple classes, one of the multiple classes corresponding to the at least one fluorescently tagged NAA recognizer.
60. In some embodiments, the techniques described herein relate to a method, wherein determining the measure of similarity between the fluorescence data and each of the multiple classes of the classification model includes: determining a fluorescence dye composition distance between the fluorescence data and each of the multiple classes to obtain fluorescence dye composition distances as the similarity measurements.
61. In some embodiments, the techniques described herein relate to a method, further including: determining, using an assignment of the fluorescently tagged NAA recognizers to the recognition segments of the plurality of reads, amino acid variant identities of the plurality of reads.
62. In some embodiments, the techniques described herein relate to a method, wherein determining, using the assignment of the fluorescently tagged NAA recognizers to the recognition segments of the plurality of reads, amino acid variant identities of the plurality of reads includes: determining the amino acid variant identities of the plurality of reads using a trained machine learning model.
63. In some embodiments, the techniques described herein relate to a method, wherein the trained machine learning model includes a classification model, and the method further includes training the classification model by: clustering the plurality of reads to obtain multiple classes each corresponding to a particular amino acid variant, wherein determining the amino acid variant identities of the plurality of reads using the trained machine learning model includes: classifying each of at least some of the plurality of reads into one of the classes to obtain an amino acid variant identity of the read.
64. In some embodiments, the techniques described herein relate to a method, wherein clustering the plurality of reads to obtain the multiple classes includes: clustering the plurality of reads using at least one of: dynamic time warping or k-means clustering.
65. In some embodiments, the techniques described herein relate to a system for identifying amino acid residues in peptides using data obtained by a sequencing device from detecting light emissions by fluorescently tagged N-terminal amino acid (NAA) recognizers during sequencing of the peptides, the method including: a sequencing device, the sequencing device configured to obtain sequencing data generated from traces of light pulses output by the sequencing device from detecting light emissions by fluorescently tagged N-terminal amino acid (NAA) recognizers in response to illumination during sequencing of at least one peptide, the sequencing data including: light pulse durations of the light pulses; and inter-pulse durations between successive ones of the light pulses; at least one computer hardware processor; and at least one non-transitory computer-readable storage medium storing instructions that, when executed by the at least one computer hardware processor, cause the at least one computer hardware processor to: generate, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assign, to recognition segments in the plurality of reads, fluorescently tagged NAA recognizers determined to be binding in the recognition segments.
66. In some embodiments, the techniques described herein relate to a system, wherein assigning, to the recognition segments in the sequence of recognition segments, the fluorescently tagged NAA recognizers determined to be binding in the recognition segments includes, for each of the recognition segments: obtain fluorescence data for the recognition segment, the fluorescence data indicating detected fluorescence intensity and fluorescence decay of at least one fluorescent dye of at least one fluorescently tagged NAA recognizer binding in the recognition segment; and identify, using the fluorescence data for the recognition segment, the at least one fluorescently tagged NAA recognizer from among a set of candidate fluorescently tagged NAA recognizers for assignment to the recognition segment.
67. In some embodiments, the techniques described herein relate to a system, wherein identifying, using the fluorescence data for the recognition segment, the at least one fluorescently tagged NAA recognizer from among the set of candidate fluorescently tagged NAA recognizers for assignment to the recognition segment includes: process, using a classification model trained to classify fluorescence data as corresponding to one of the set of candidate fluorescently tagged NAA recognizers, the fluorescence data for the recognition segment to determine a classification of the fluorescence data as corresponding to the at least one fluorescently tagged NAA recognizer.
68. In some embodiments, the techniques described herein relate to a system, wherein the classification model includes a Gaussian Mixture Model (GMM) classifier.
69. In some embodiments, the techniques described herein relate to a system, wherein the instructions further cause the at least one computer hardware processor to train the classification model, the training including: obtain fluorescence data for the light pulses emitted by the fluorescently tagged NAA recognizers during the sequencing of the at least one peptide; and train the classification model using the fluorescence data.
70. In some embodiments, the techniques described herein relate to a system, wherein obtaining the fluorescence data for the light pulses includes, for each of multiple ones of the light pulses: log of fluorescence intensity detected after illumination that caused emission of the light pulse; and a ratio between: (1) a number of photons detected in a first time bin after the illumination, and (2) a number of photons detected in a second time bin after the illumination, wherein the second time bin is subsequent to the first time bin.
71. In some embodiments, the techniques described herein relate to a system, wherein processing, using the classification model, the fluorescence data to determine the classification of the fluorescence data as corresponding to the at least one fluorescently tagged NAA recognizer includes: determining a measure of similarity between the fluorescence data and each of multiple classes of the classification model, the multiple classes each corresponding to at least one candidate fluorescently tagged NAA recognizer of the candidate fluorescently tagged NAA recognizers; and selecting, as the classification, using similarity measurements determined for the multiple classes, one of the multiple classes corresponding to the at least one fluorescently tagged NAA recognizer.
72. In some embodiments, the techniques described herein relate to a system, wherein determining the measure of similarity between the fluorescence data and each of the multiple classes of the classification model includes: determine a fluorescence dye composition distance between the fluorescence data and each of the multiple classes to obtain fluorescence dye composition distances as the similarity measurements.
73. In some embodiments, the techniques described herein relate to a non-transitory computer-readable storage medium storing instructions that, when executed by at least one computer hardware processor, cause the at least one computer hardware processor to perform a method for identifying amino acid residues in peptides using data obtained by a sequencing device from detecting light emissions by fluorescently tagged N-terminal amino acid (NAA) recognizers during sequencing of the peptides, the method including: obtaining sequencing data generated from traces of light pulses output by the sequencing device from detecting light emissions by fluorescently tagged NAA recognizers in response to illumination during sequencing of at least one peptide, the sequencing data including: light pulse durations of the light pulses; and inter-pulse durations between successive ones of the light pulses; generating, using the light pulse durations and the inter-pulse durations, a plurality of reads, the plurality of reads each including a sequence of recognition segments that each indicate a particular time period in which one or more of the fluorescently tagged NAA recognizers were binding to a particular NAA of the at least one peptide; and assigning, to recognition segments in the plurality of reads, fluorescently tagged NAA recognizers determined to be binding in the recognition segments.
74. In some embodiments, the techniques described herein relate to a non-transitory computer-readable storage medium, wherein assigning, to the recognition segments in the sequence of recognition segments, the fluorescently tagged NAA recognizers determined to be binding in the recognition segments includes, for each of the recognition segments: obtaining fluorescence data for the recognition segment, the fluorescence data indicating detected fluorescence intensity and fluorescence decay of at least one fluorescent dye of at least one fluorescently tagged NAA recognizer binding in the recognition segment; and identifying, using the fluorescence data for the recognition segment, the at least one fluorescently tagged NAA recognizer from among a set of candidate fluorescently tagged NAA recognizers for assignment to the recognition segment.
75. In some embodiments, the techniques described herein relate to a non-transitory computer-readable storage medium, wherein identifying, using the fluorescence data for the recognition segment, the at least one fluorescently tagged NAA recognizer from among the set of candidate fluorescently tagged NAA recognizers for assignment to the recognition segment includes: processing, using a classification model trained to classify fluorescence data as corresponding to one of the set of candidate fluorescently tagged NAA recognizers, the fluorescence data for the recognition segment to determine a classification of the fluorescence data as corresponding to the at least one fluorescently tagged NAA recognizer.
76. In some embodiments, the techniques described herein relate to a non-transitory computer-readable storage medium, wherein the classification model includes a Gaussian Mixture Model (GMM) classifier.

### Example Computing Device

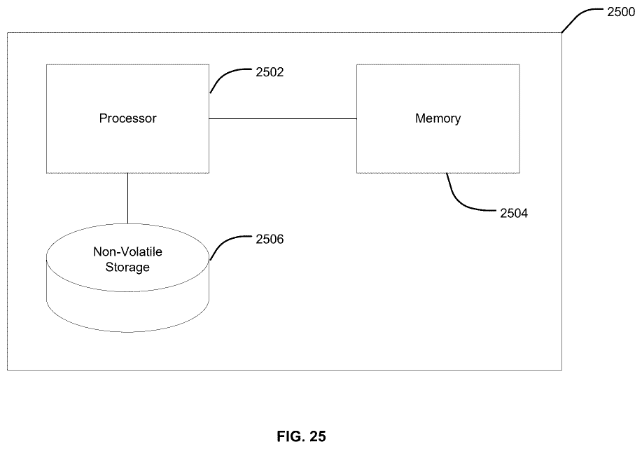

FIG. 25 illustrates a block diagram of a computing device 2500 that can be specially configured to implement some embodiments of the technology described herein. The computing device 2500 may include one or more computer hardware processors 2502 and non-transitory computer-readable storage media (e.g., memory 2504 and one or more non-volatile storage devices 2506). The processor(s) 2502 may control writing data to and reading data from (1) the memory 2504; and (2) the non-volatile storage device(s) 2506. To perform any of the functionality described herein, the processor(s) 2502 may execute one or more processor-executable instructions stored in one or more non-transitory computer-readable storage media (e.g., the memory 2504), which may serve as non-transitory computer-readable storage media storing processor-executable instructions for execution by the processor(s) 2502.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method for detecting amino acid variants in peptides using data obtained by a sequencing device, the method comprising:
using at least one computer hardware processor to perform:
obtaining sequencing data generated from traces of light pulses output by the sequencing device from detecting light emissions by fluorescently tagged N-terminal amino acid (NAA) recognizers in response to illumination during sequencing of at least one peptide, the sequencing data comprising:
light pulse durations of the light pulses; and
inter-pulse durations between successive ones of the light pulses;
generating, using the light pulse durations and the inter-pulse durations, a plurality of reads, the plurality of reads each comprising a sequence of recognition segments that each indicate a particular time period in which one or more of the fluorescently tagged NAA recognizers were binding to a particular NAA of the at least one peptide;
assigning, to recognition segments in the plurality of reads, fluorescently tagged NAA recognizers determined to be binding in the recognition segments at least in part by using: (1) the light pulse durations, and (3) the inter-pulse durations; and
detecting one or more amino acid variants in the at least one peptide using the plurality of reads and an assignment of the fluorescently tagged NAA recognizers to the recognition segments of the plurality of reads.

2. The method of claim 1, wherein assigning, to the recognition segments in the plurality of reads, the fluorescently tagged NAA recognizers determined to be binding in the recognition segments comprises, for each of the recognition segments:
obtaining fluorescence data for the recognition segment, the fluorescence data indicating detected fluorescence intensity and fluorescence decay of at least one fluorescent dye of at least one fluorescently tagged NAA recognizer binding in the recognition segment; and
identifying, using the fluorescence data for the recognition segment, the at least one fluorescently tagged NAA recognizer from among a set of candidate fluorescently tagged NAA recognizers for assignment to the recognition segment.

3. The method of claim 1, wherein detecting the one or more amino acid variants in the at least one peptide using the plurality of reads and the assignment of the fluorescently tagged NAA recognizers to the recognition segments of the plurality of reads comprises:
aligning at least one of the plurality of reads to each of one or more reference peptide sequences to obtain one or more peptide alignments at least in part by using the assignment of the fluorescently tagged NAA recognizers to the recognition segments of the plurality of reads; and
performing the detecting of the one or more amino acid variants using the one or more peptide alignments.

4. The method of claim 3, wherein aligning the at least one read to each of one or more reference peptide sequences comprises:
accessing, for amino acid residues in the reference peptide sequence, expected fluorescently tagged NAA recognizers; and
assigning recognition segments in the at least one read to amino acid residues in the reference peptide sequence at least in part by matching fluorescently tagged NAA recognizers assigned to the recognition segments to expected fluorescently tagged NAA recognizers of the amino acid residues in the reference peptide sequence.

5. The method of claim 3, wherein aligning the at least one read to each of the one or more reference peptide sequences comprises:
generating multiple candidate alignments with the reference peptide sequence; and
determining alignment scores for the candidate alignments; and
selecting, using the alignment scores determined for the candidate alignments, one of the candidate alignments as an alignment of the at least one peptide with the reference peptide sequence.

6. The method of claim 5, wherein determining the alignment scores the candidate alignments comprises, for each of the candidate alignments:
obtaining expected light pulse durations for amino acid residues in the reference peptide sequence;
comparing light pulse durations of recognition segments in the at least one read to expected light pulse durations of respective amino acid residues in the reference peptide sequence with which the recognition segments are aligned; and
determining an alignment score for the candidate alignment using a result of comparing the light pulse durations of the recognition segments to the expected light pulse durations of the respective amino acid residues in the reference peptide sequence.

7. The method of claim 6, wherein obtaining the expected light pulse durations for the amino acid residues in the reference peptide sequence comprises:
accessing a reference dataset storing pulse durations for amino acid motifs; and
determining, using the pulse durations for the amino acid motifs, the expected light pulse durations for the amino acid residues in the reference peptide sequence.

8. The method of claim 7, wherein determining, using the pulse durations for the amino acid motifs, the expected light pulse durations for the amino acid residues in the reference peptide sequence comprises, for each of at least some of the amino acid residues in the reference peptide sequence:
identifying a subsequence of the reference peptide consisting of the amino acid residue and one or more preceding amino acid residues;
identifying, in the reference dataset, one of the amino acid motifs using the subsequence; and
determining, as an expected pulse duration for the amino acid residue, a pulse duration stored for the identified amino acid motif in the reference dataset.

9. The method of claim 8, further comprising determining at least some of the pulse durations stored in the reference dataset for at least some of the amino acid motifs using a trained machine learning model to predict pulse durations at least in part by:
generating sets of features for the at least some amino acid motifs; and
providing the sets of features as input to the machine learning model to obtain output indicating the at least some pulse durations for the at least some amino acid motifs.

10. The method of claim 9, wherein generating the sets of features for the at least some amino acid motifs comprises, for each of the at least some amino acid motifs:
generating a one-hot encoding of amino acids in the amino acid motif;
generating a sinusoidal positional encoding of amino acid positions in the amino acid motif; and
generating a set of features for the amino acid motif at least in part by combining the one-hot encoding and the sinusoidal positional encoding.

11. The method of claim 10, wherein the trained machine learning model comprises a neural network, the neural network comprising:
a plurality of fully connected layers comprising:
a first layer configured to receive a combination of an input one-hot encoding with an input sinusoidal positional encoding generated for a particular amino acid motif; and
an output layer configured to output a pulse duration prediction for the particular amino acid motif.

12. The method of claim 6, wherein comparing the light pulse durations of the recognition segments to the expected light pulse durations of the respective amino acid residues in the reference peptide sequence comprises:
determining differences between mean light pulse durations of the recognition segments and the expected light pulse durations of the respective amino acid residues in the reference peptide sequence; and
determining a component of the alignment score using the differences between the mean light pulse durations of the recognition segments and the expected light pulse durations of the respective amino acid residues in the reference peptide sequence.

13. The method of claim 6, wherein determining the alignment scores for the candidate alignments comprises, for each of the candidate alignments:
identifying positions in the candidate alignment where amino acid residues of the reference peptide sequence have expected fluorescently tagged NAA recognizers but are not aligned with any recognition segment in the at least one read; and
determining an alignment score for the candidate alignment based on the identified positions.

14. The method of claim 13, wherein determining the alignment score for the candidate alignment based on the identified positions comprises:
accessing expected pulse durations for the amino acid residues of the reference peptide sequence at the identified positions;
determining a deletion penalty using the expected pulse durations of the amino acid residues of the reference peptide sequence; and
determining the alignment score for the candidate alignment using the deletion penalty.

15. The method of claim 6, wherein determining the alignment scores for the candidate alignments comprises, for each of the candidate alignments:
determining a gap score for the candidate alignment based on spacing between recognition segments of the at least one read relative to the reference peptide sequence; and
determining an alignment score for the candidate alignment using the gap score.

16. The method of claim 1, wherein detecting the one or more amino acid variants in the at least one peptide using the plurality of reads and the assignment of the fluorescently tagged NAA recognizers to the recognition segments of the plurality of reads comprises:
determining, using the assignment of the fluorescently tagged NAA recognizers to the recognition segments of the plurality of reads, amino acid variant identities of the plurality of reads.

17. The method of claim 16, wherein determining, using the assignment of the fluorescently tagged NAA recognizers to the recognition segments of the plurality of reads, amino acid variant identities of the plurality of reads comprises:
determining the amino acid variant identities of the plurality of reads using a trained machine learning model.

18. The method of claim 17, wherein the trained machine learning model comprises a classification model, and the method further comprises training the classification model by:
clustering the plurality of reads to obtain multiple classes each corresponding to a particular amino acid variant, wherein determining the amino acid variant identities of the plurality of reads using the trained machine learning model comprises:
classifying each of at least some of the plurality of reads into one of the classes to obtain an amino acid variant identity of the read.

19. A system for identifying amino acid variants in peptides using data obtained by a sequencing device, the system comprising:
the sequencing device, the sequencing device configured to obtain sequencing data generated from traces of light pulses output by the sequencing device from detecting light emissions by fluorescently tagged N-terminal amino acid (NAA) recognizers in response to illumination during sequencing of at least one peptide, the sequencing data comprising:
light pulse durations of the light pulses; and
inter-pulse durations between successive ones of the light pulses;
at least one computer hardware processor; and
at least one non-transitory computer-readable storage medium storing instructions that, when executed by the at least one computer hardware processor, cause the at least one computer hardware processor to:
generate, using the light pulse durations and the inter-pulse durations, a plurality of reads, the plurality of reads each comprising a sequence of recognition segments that each indicate a particular time period in which one or more of the fluorescently tagged NAA recognizers were binding to a particular NAA of the at least one peptide;
assign, to recognition segments in the plurality of reads, fluorescently tagged NAA recognizers determined to be binding in the recognition segments at least in part by using: (1) the light pulse durations, and (3) the inter-pulse durations; and
detect one or more amino acid variants in the at least one peptide using the plurality of reads and an assignment of the fluorescently tagged NAA recognizers to the recognition segments of the plurality of reads.

20. A non-transitory computer-readable storage medium storing instructions that, when executed by at least one computer hardware processor, cause the at least one computer hardware processor to perform a method for identifying amino acid variants in peptides using data obtained by a sequencing device, the method comprising:
obtaining sequencing data generated from traces of light pulses output by the sequencing device from detecting light emissions by fluorescently tagged N-terminal amino acid (NAA) recognizers in response to illumination during sequencing of at least one peptide, the sequencing data comprising:
light pulse durations of the light pulses; and
inter-pulse durations between successive ones of the light pulses;
generating, using the light pulse durations and the inter-pulse durations, a plurality of reads, the plurality of reads each comprising a sequence of recognition segments that each indicate a particular time period in which one or more of the fluorescently tagged NAA recognizers were binding to a particular NAA of the at least one peptide;
assigning, to recognition segments in the plurality of reads, fluorescently tagged NAA recognizers determined to be binding in the recognition segments at least in part by using: (1) the light pulse durations, and (3) the inter-pulse durations; and
detecting one or more amino acid variants in the at least one peptide using the plurality of reads and an assignment of the fluorescently tagged NAA recognizers to the recognition segments of the plurality of reads.
